SEQUENCE LISTING (42 sequences)

SEQ ID NO: 1 - DNA, 25 bp, Homo sapiens
  source 1..25: mol_type = genomic DNA
gcccttctattaaaacacagacaca

SEQ ID NO: 2 - DNA, 25 bp, Mus sp.
  source 1..25: mol_type = genomic DNA
gcccttctattgaaacacagataca

SEQ ID NO: 3-4 - skipped (2 entries)

SEQ ID NO: 5 - DNA, 11 bp, synthetic construct
  source 1..11: mol_type = other DNA
aaaaacagtac

SEQ ID NO: 6 - RNA, 25 nt, Homo sapiens
  source 1..25: mol_type = genomic RNA
tgtgtctgtgttttaatagaagggc

SEQ ID NO: 7-8 - skipped (2 entries)

SEQ ID NO: 9 - RNA, 11 nt, Homo sapiens
  source 1..11: mol_type = genomic RNA
gtactgttttt

SEQ ID NO: 10 - protein, 12 aa, Human immunodeficiency virus 1
GRKKRRQRRRPQ

SEQ ID NO: 11 - DNA, 21 bp, synthetic construct
  source 1..21: mol_type = other DNA
agacgagattcgatcggagta

SEQ ID NO: 12 - DNA, 21 bp, synthetic construct
  source 1..21: mol_type = other DNA
aaggccacactgactgtagac

SEQ ID NO: 13 - DNA, 21 bp, synthetic construct
  source 1..21: mol_type = other DNA
tggccaccagattcttatcag

SEQ ID NO: 14 - DNA, 20 bp, synthetic construct
  source 1..20: mol_type = other DNA
agcccagtgttaccaccaag

SEQ ID NO: 15 - DNA, 20 bp, synthetic construct
  source 1..20: mol_type = other DNA
acccaagaacaagcacaagg

SEQ ID NO: 16 - DNA, 30 bp, synthetic construct
  source 1..30: mol_type = other DNA
ataaaaggatttgatactcatctgctagag

SEQ ID NO: 17 - DNA, 23 bp, synthetic construct
  source 1..23: mol_type = other DNA
tgtcatccacttccaagaactcc

SEQ ID NO: 18 - DNA, 23 bp, synthetic construct
  source 1..23: mol_type = other DNA
aagccagaccatggatactggag

SEQ ID NO: 19 - DNA, 23 bp, synthetic construct
  source 1..23: mol_type = other DNA
aactggagaatagaacaccagag

SEQ ID NO: 20 - DNA, 25 bp, synthetic construct
  source 1..25: mol_type = other DNA
tgcatctttccaccagttccggggc

SEQ ID NO: 21 - DNA, 23 bp, synthetic construct
  source 1..23: mol_type = other DNA
tcaagttgctctttgttgtcaac

SEQ ID NO: 22 - DNA, 23 bp, synthetic construct
  source 1..23: mol_type = other DNA
ttgtatttgcttggagagccagt

SEQ ID NO: 23 - DNA, 21 bp, synthetic construct
  source 1..21: mol_type = other DNA
tcgctgctgtcctccgagtcc

SEQ ID NO: 24 - DNA, 22 bp, synthetic construct
  source 1..22: mol_type = other DNA
ggtttgcctcttctccacagac

SEQ ID NO: 25 - DNA, 22 bp, synthetic construct
  source 1..22: mol_type = other DNA
cctgtggatgactgagtacctg

SEQ ID NO: 26 - DNA, 23 bp, synthetic construct
  source 1..23: mol_type = other DNA
agccaggagaaatcaaacagagg

SEQ ID NO: 27 - DNA, 18 bp, synthetic construct
  source 1..18: mol_type = other DNA
aaatgtccgctgggccaa

SEQ ID NO: 28 - DNA, 19 bp, synthetic construct
  source 1..19: mol_type = other DNA
catcgacttcgtacaaggg

SEQ ID NO: 29 - DNA, 23 bp, synthetic construct
  source 1..23: mol_type = other DNA
gccgcactctttctgactacaac

SEQ ID NO: 30 - DNA, 22 bp, synthetic construct
  source 1..22: mol_type = other DNA
acctccagagtgatggtcttgc

SEQ ID NO: 31 - DNA, 20 bp, synthetic construct
  source 1..20: mol_type = other DNA
tccaggtatgtgggtttcat

SEQ ID NO: 32 - DNA, 22 bp, synthetic construct
  source 1..22: mol_type = other DNA
gatttgatgctcatctgttgga

SEQ ID NO: 33 - DNA, 20 bp, synthetic construct
  source 1..20: mol_type = other DNA
tggactgtggtcaatgttgc

SEQ ID NO: 34 - DNA, 24 bp, synthetic construct
  source 1..24: mol_type = other DNA
gatagtgaggaccagcatctaatg

SEQ ID NO: 35 - DNA, 20 bp, synthetic construct
  source 1..20: mol_type = other DNA
caacatcaaggggtcacctc

SEQ ID NO: 36 - DNA, 22 bp, synthetic construct
  source 1..22: mol_type = other DNA
catgaatgatacaaccgtgtgg

SEQ ID NO: 37 - DNA, 21 bp, synthetic construct
  source 1..21: mol_type = other DNA
cctggtgctccatgaggagac

SEQ ID NO: 38 - DNA, 22 bp, synthetic construct
  source 1..22: mol_type = other DNA
cagactctgaccttttgccagg

SEQ ID NO: 39 - DNA, 22 bp, synthetic construct
  source 1..22: mol_type = other DNA
gaggagcaaaccaaacggcttc

SEQ ID NO: 40 - DNA, 22 bp, synthetic construct
  source 1..22: mol_type = other DNA
aaggcgagactcttcatcagcc

SEQ ID NO: 41 - DNA, 22 bp, synthetic construct
  source 1..22: mol_type = other DNA
atcgccctgtggatgactgagt

SEQ ID NO: 42 - DNA, 23 bp, synthetic construct
  source 1..23: mol_type = other DNA
gccaggagaaatcaaacagaggc

(19) United States
(12) Patent Application Publication
(10) Pub. No.: US 2026/0176692 A1
(43) Pub. Date: Jun. 25, 2026

(54) METHODS FOR TREATING AND PREVENTING B CELL DISORDERS

(71) Applicants: The Regents of the University of California, Oakland, CA (US); City of Hope, Duarte, CA (US)

(72) Inventors: Ameae M. Walker, Riverside, CA (US); Srividya Swaminathan, Duarte, CA (US)

(73) Assignees: The Regents of the University of California, Oakland, CA (US); City of Hope, Duarte, CA (US)

(21) Appl. No.: 19/125,426

(22) PCT Filed: Oct. 27, 2023
(86) PCT No.: PCT/US2023/036128

§ 371 Date: Apr. 29, 2025

Related U.S. Application Data
(60) Provisional application No. 63/421,427, filed on Nov. 1, 2022.

(51) Int. Cl. C12Q 1/6883; C12N 15/113; C12Q 1/6886

(52) U.S. Cl. CPC C12Q 1/6883; C12N 15/113; C12Q 1/6886

(57) ABSTRACT

Certain embodiments of the invention provide a method of preventing or treating a B cell mediated disorder, such as B cell lymphoma and/or lupus.

### CROSS REFERENCE TO RELATED APPLICATION

This application claims priority to U.S. Provisional Application No. 63/421,427 that was filed on Nov. 1, 2022. The entire content of the application referenced above is hereby incorporated by reference herein.

### GOVERNMENT FUNDING

This invention was made with government support under CA107399, CA033572, and CA276517 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Feb. 14, 2024, is named 12111_043WO1_SL and is 35,288 bytes in size.

### BACKGROUND OF THE INVENTION

B cells (B lymphocytes) are one type of white blood cell that make antibodies to fight infections and are an important part of the immune/lymphatic system. Debilitating or lethal B cell disorders, such as autoimmune diseases involving autoreactive B cells and B cell malignancies, remain extremely challenging to control. Diffuse large B cell lymphoma (DLBCL) is the most common type of non-Hodgkin lymphoma (NHL) in the United States and worldwide, accounting for about 22 percent of newly diagnosed cases of B cell NHL in the United States. More than 18,000 people are diagnosed with DLBCL each year. DLBCL is an aggressive, fast-growing lymphoma. Although it can occur in childhood, the occurrence of DLBCL generally increases with age, and most patients are over the age of 60 at diagnosis.

Meanwhile, autoimmune diseases, such as systemic lupus erythematosus (SLE; lupus), are also notoriously intractable to pharmacotherapy. Diagnosed or suspected lupus patients have auto-antibodies secreted from the abnormal expansion of autoreactive B cell clones against autologous antigens. Lupus is correlated with an increased risk of non-Hodgkin lymphoma, and the risk is estimated to be about 4-7 times greater than that in the general population. In addition, diffuse large B cell lymphoma (DLBCL) is reported to account for about 37-62% of all lymphomas that develop in lupus patients.

The most common current treatment for advanced DLBCL is called R-CHOP. This treatment combines three chemotherapy drugs with an antibody and a steroid, including Rituximab (Rituxan), Cyclophosphamide (Cytoxan), Hydroxydaunorubicin (Doxorubicin), Vincristine (Oncovin), and Prednisone. The R-CHOP protocol has all the well-known disadvantages of general chemotherapy, including nausea, hair loss, susceptibility to infection, malaise, and widespread and irreversible toxicities, such as nerve damage and cognitive decline. Treatment frequently may also include radiation, which can cause burns, nerve pain, hair loss and general malaise.

For recurrent DLBCL, additional therapeutic approaches may include: 1) CAR-T therapies, such as Axicabtagene ciloleucel (Yescarta) or Tisagenlecleucel (Kymriah); 2) a checkpoint inhibitor immunotherapy, such as Pembrolizumab (Keytruda); and/or 3) Polatuzumab vedotin (Polivy), which latter binds to the surface of DLBCL cells and delivers a tumoricidal drug to the cancer cells. CAR-T therapies are custom tailored therapies, and therefore, are high cost. Additionally, CAR-T therapies have been shown to induce cytokine storms, which can be life-threatening. Checkpoint inhibitors, such as Keytruda, also have been associated with cytokine storms, as well as the development of autoimmune diseases, whereas Polivy has been reported to cause severe pain and brain infections.

Accordingly, new preventative and/or therapeutic interventions are needed to manage B cell mediated disorders, such as interventions that are more efficacious and/or have reduced side effects.

### SUMMARY OF THE INVENTION

Certain embodiments provide a method of treating or preventing a B cell mediated disorder in a subject in need thereof, comprising administering a splice modulating oligonucleotide (SMO) as described herein to the subject, wherein the SMO inhibits B cell expression of the long form prolactin receptor (LF PRLR) and/or intermediate form (IF) PRLR; and/or reduces the relative B cell expression of LF/IF PRLR compared to B cell expression of one or more short form prolactin receptors (SF PRLR). For example, in certain embodiments, the SMO reduces the relative expression of LF/IF PRLR over total PRLR expression (e.g., LF+IF+SF; see Example 1). In certain embodiments, the method further comprises administering one or more additional therapeutic agents described herein (e.g., an immune cell therapy).

Certain embodiments provide a splice modulating oligomer (SMO) as described herein for the prophylactic or therapeutic treatment of a B cell mediated disorder. In certain embodiments, such a SMO may be used in combination with one or more additional therapeutic agents described herein (e.g., an immune cell therapy).

Certain embodiments provide the use of a SMO as described herein to prepare a medicament for the treatment of a B cell mediated disorder in a mammal. In certain embodiments, such a treatment may be in combination with one or more additional therapeutic agents described herein (e.g., an immune cell therapy).

Certain embodiments provide a composition comprising a SMO as described herein for the prophylactic or therapeutic treatment of a B cell mediated disorder. In certain embodiments, the composition further comprises one or more additional therapeutic agents as described herein.

Certain embodiments provide the use of a composition comprising a SMO as described herein to prepare a medicament for the treatment of a B cell mediated disorder in a mammal. In certain embodiments, the composition further comprises one or more additional therapeutic agents as described herein.

Certain embodiments provide a method of reducing a subject's likelihood of developing a B cell malignancy, wherein the subject has an autoimmune disease or has premalignant B cell clone(s), the method comprising administering a SMO as described herein to the subject, wherein the SMO inhibits B cell expression of the long form PRLR (LF PRLR) and/or IF PRLR; and/or reduces the relative B cell expression of LF/IF PRLR compared to B cell expression of one or more short form prolactin receptors (SF PRLR). For example, in certain embodiments, the SMO reduces the relative expression of LF/IF PRLR over total PRLR expression (e.g., LF+IF+SF, see Example 1).

Certain embodiments provide a SMO as described herein for reducing a subject's likelihood of developing a B cell malignancy, wherein the subject has an autoimmune disease or has premalignant B cell clone(s).

Certain embodiments provide the use of a SMO as described herein to prepare a medicament reducing a subject's likelihood of developing a B cell malignancy, wherein the subject has an autoimmune disease or has premalignant B cell clone(s).

Certain embodiments provide a method of identifying a subject as having a higher likelihood of developing a B cell malignancy, the method comprising detecting the expression level of prolactin or LF/IF PRLR in a sample from the subject, wherein the subject is identified as having a higher likelihood of developing a B cell malignancy when the expression level of prolactin or LF/IF PRLR is higher than a control reference level.

Certain embodiments provide a method of identifying a subject as a candidate for treatment with a B cell malignancy therapy, comprising detecting the expression level of prolactin or LF/IF PRLR in a sample from the subject, wherein the subject is identified as a candidate for treatment when the expression level of prolactin or LF/IF PRLR is higher than a control reference level.

Certain embodiments provide a method of reducing the number of B cells (e.g., B cells that overexpress LF/IF PRLR; or pathogenic, autoreactive B cells, such as pathogenic, autoreactive plasma cells and/or their precursor cells) in a subject in need thereof (e.g., a subject having autoimmune disorder, such as SLE), comprising administering a SMO as described herein to the subject. In certain embodiments, the number of normal, non-autoreactive B cells is not reduced.

Certain embodiments provide a method of identifying a subject as a candidate for treatment with a SLE therapy, comprising detecting the expression level of LF/IF PRLR or total PRLR in a sample (e.g., PBMCs, or B cells such as plasma cells) from the subject, wherein the subject is identified as a candidate for treatment when the expression level of LF/IF PRLR or total PRLR is higher than a control reference level.

### BRIEF DESCRIPTION OF THE FIGURES

FIGS. 1A-1F: Knockdown of LFPRLR reduces splenic B-cell subsets in SLE-prone mice. (FIG. 1a-c) Experimental design (FIG. 1a), verification of LFPRLR knockdown in total splenic WBCs by qPCR (FIG. 1b), and splenic WBC counts (FIG. 1c) in MRL-lpr mice treated with control SMO [n=9 for (FIG. 1b) and n=14 for (FIG. 1c)] or LFPRLR SMO [n=10 for (FIG. 1b) and n=14 for (FIG. 1c)]. (FIG. 1d-f) Quantitation and representative flow cytometry plots of CD3⁻CD19⁺ B cells and CD19⁻CD3⁺ T cells (FIG. 1d); CD11c⁺PDCA1⁺ pDCs and CD11c⁺PDCA1⁻ cDCs (FIG. 1e); Blimp1⁺CD138⁺ total plasma cells, Blimp1⁺CD138⁺B220⁺ plasmablasts, and Blimp1⁺CD138⁺B220⁻ long lived plasma cells (FIG. 1f) in spleens of MRL-lpr mice treated with control SMO (n=14) or LFPRLR SMO (n=14). Graphs show median interquartile range. Each dot in qPCR analyses of transcripts corresponds to the mean expression of that gene in one mouse calculated from 3 technical replicates. Ubiquitin (UB) was used as the house-keeping gene in qPCR. Exact p-values were calculated using the Mann-Whitney U test. ns=non-significant. As used herein, “LFPRLR SMO” may also be referenced as “Antimaia”, wherein the mouse form of Antimaia corresponds to mLFPRLR SMO and the human form of Antimaia corresponds to hLFPRLR SMO (see, Example 1, Table s1a).

FIGS. 2A-2F: Knockdown of LFPRLR in SLE-prone mice decreases factors associated with the risk of lymphoma initiation. (FIG. 2a-d) Verification of LFPRLR knockdown in isolated B cells by qPCR (FIG. 2a), percentages of B cells in sub G0/G1, G0/G1, S, and G2/M phases with representative flow cytometry plots (FIG. 2b), transcript levels of MYC, BCL2 by qPCR (FIG. 2c), and BCL2 protein by flow cytometry (FIG. 2d) in spleens of MRL-lpr mice treated with control SMO (n=14) or LFPRLR SMO (n=14). (FIG. 2e) Next-generation sequencing of total IGH repertoire (GSE207186) to compare spectratype of splenic B cells (left); and frequencies of splenic B cells with IGH CDR3 ≥20aa (middle), and frequencies of B cells with non-productive IGH rearrangements (right) in MRL-lpr SLE-prone mice treated with control (n=6) or LFPRLR SMO (n=6). (FIG. 2f) AID mRNA (left) and protein levels in splenic B cells (middle) and representative histograms for AID protein staining (right) in control SMO-treated (n=14) and LFPRLR SMO-treated (n=14) MRL-lpr mice by flow cytometry. Splenic WBCs from wildtype mice cultured with IL-4 and LPS were used as positive controls for AID staining. Each dot in qPCR analyses of transcripts corresponds to the mean expression of that gene in one mouse calculated from 3 technical replicates. UB was used as the house-keeping gene in qPCR. Graphs show median±interquartile range. Exact p-values were calculated using the Mann-Whitney U test. ns=non-significant, FMO=fluorescence minus one control, MFI=median fluorescence intensity. In the graphs shown in FIGS. 2c and 2f, “Control SMO” is shown on the left and “LFPRLR SMO” is shown on the right.

FIGS. 3A-3C: Knockdown of LFPRLR impacts early B-lymphopoiesis in SLE-prone mice. (FIG. 3a) Quantitation and representative flow cytometry plots of bone marrow CD19⁺ B cells in MRL-lpr mice treated with control SMO (n=9) or LFPRLR SMO (n=9). (FIG. 3b) Quantitation and representative histograms showing median fluorescence intensity (MFI) of BCL2 protein in bone marrow B cells of control SMO-treated (n=5) and LFPRLR SMO-treated (n=5) MRL-lpr mice by flow cytometry. (FIG. 3c) Schematic depicting the mechanisms by which LFPRLR increases the number of abnormal splenic B cells available for potential malignant transformation or the concurrent worsening of autoimmune pathology in SLE-prone mice. Graphs show median±interquartile range. Exact p-values were calculated using the Mann-Whitney U test. ns=non-significant, FMO=fluorescence minus one control.

FIGS. 4A-4I: Knockdown of LFPRLR in DLBCL-prone mice suppresses factors that can drive overt B-cell lymphomagenesis. (FIG. 4a-c) Experimental design (FIG. 4a), verification of LFPRLR knockdown in total splenic WBCs by qPCR (FIG. 4b), and splenic WBC counts (c) in TCL1-tg DLBCL-prone mice treated with control SMO [n=8 for (FIG. 4b) and n=10 for (c)] or LFPRLR SMO to knockdown LFPRLR [n=8 for (FIG. 4b) and n=10 for (FIG. 4c)]. (FIG. 4d-h) Quantitation and representative flow cytometry plots of CD3⁻CD19⁺ B cells (FIG. 4d,g); and B cell-specific expression of BCL2 (FIG. 4e,f,h) in spleen and bone marrow of TCL1-tg mice treated with control SMO (n=8) or LFPRLR SMO (n=8). One representative histogram from each group for measurement of BCL2 protein in spleen and bone marrow is shown. (FIG. 4i) PRL-LFPRLR signaling upregulates BCL2 and increases survival of pre-malignant B cells, thereby enhancing the risk of establishment of overt B-cell malignancies. Graphs show median±interquartile range. Each dot in qPCR analyses of transcripts corresponds to the mean expression of that gene in one mouse calculated from 3 technical replicates. UB was used as the house keeping gene in qPCR. Exact p-values were calculated using the Mann-Whitney U test. ns=non-significant, FMO=fluorescence minus one control, MFI=median fluorescence intensity.

FIGS. 5A-5J: PRL-LFPRLR signaling maintains overt human B-cell malignancies. (FIG. 5a) Overall survival probabilities of patients with B-cell lymphomas from 3 datasets (GSE4475, GSE10846, and E-TABM-346) divided into PRL^{high} and PRL^{low} based on the median expression of PRL mRNA. GSE4475 includes 123 DLBCL and 36 Burkitt lymphoma patients at diagnosis. GSE10846 includes samples from 414 DLBCL patients collected after treatment with rituximab-CHOP or CHOP, and E-TABM-346 includes samples from 53 DLBCL patients collected after treatment with rituximab-CHOP or CHOP. (FIG. 5b-c) Expression of PRL mRNA in CD3⁻CD19⁻ B cells from healthy donors (n=27) and B-lymphoblasts of patients with high-grade MYC/BCL2-driven B-ALL (n=18) (EGAS00001003266, St. Jude, median±interquartile range, p-value calculated by Mann-Whitney U test) (FIG. 5b); and in CD3⁻CD19⁺ B cells isolated from PBMC of healthy donors (n=3) and in malignant human B-cell lines (Karpas-422, SU-DHL-6, VAL and OCI-LY18, (c)). (FIG. 5d) Expression of LF/IFPRLR and SFPRLR mRNA in CD3⁻CD19⁺ B cells, CD3⁻CD56⁺ NK cells and CD19⁻CD3⁺ T cells sorted from PBMC of healthy donors (n=3) and malignant B-cell lines (Karpas-422, SU-DHL-6, VAL and OCI-LY18) (FIG. 5d). (FIG. 5e-h) MTS assay to compare viability of PBMC isolated from healthy donors after treatment with either control SMO or LFPRLR SMO for 48 h (FIG. 5e), and flow cytometry to compare the viability (% FSC^{high}DAPI⁻) of MACS-sorted CD3⁻CD19⁺ B cells (FIG. 5f), CD3⁻CD56⁺ NK cells (FIG. 5g), and CD3⁺ T cells (FIG. 5h) isolated from PBMC of four healthy donors after treatment with 73 nM of either control SMO or LFPRLR SMO for 48 h. (FIG. 5i-j) Viable cell number was assessed using MTS assay (FIG. 5i) and dead cells by measuring DAPI⁺ cells by flow cytometry (FIG. 5j) in malignant human B-cell lines after treatment with control SMO or LFPRLR SMO for 48 h. P values for survival curve were measured by log-rank (Mantel-Cox) test. For all other experiments, p-values were calculated by unpaired t test from 3 independent experiments/biological replicates (±SEM), unless otherwise indicated. qPCR of each biological sample was conducted in three technical replicates. UB was used as the house-keeping gene in qPCR. For all flow cytometry-based measurements of cell death, the IC50 concentration of the Karpas-422 line was used (73 nM).

FIGS. 6A-6E: Knockdown of LFPRLR in malignant human B cells reduces BCL2 and MYC expression. (FIG. 6a-b) LFPRLR mRNA levels and expression of MYC, BCL2 and PRL transcripts in malignant human B-cell lines (Karpas-422, SU-DHL-6, VAL and OCI-LY18) treated with 73 nM of control SMO or LFPRLR SMO for 48 h. Each qPCR sample was run in 3 technical replicates. One representative of 3 independent experiments is shown. UB was used as the house keeping gene in qPCR. P-values calculated by unpaired t test from 3 independent experiments (±SEM). Cell lines highly sensitive to low concentration (73 nM) of LFPRLR SMO are shown in (FIG. 6a) and cell lines less sensitive to LFPRLR SMO at 73 nM are shown in (FIG. 6b). (FIG. 6c-d) Immunoblotting to compare the expression of MYC and BCL2 proteins in human DLBCL cell lines (Karpas-422, SU-DHL-6, VAL and OCI-LY18) treated with 73 nM of control SMO or LFPRLR SMO for 48 h. One representative of 3 independent experiments is shown. β-actin was used as the loading control in immunoblotting. Cell lines highly sensitive to low concentration (73 nM) of LFPRLR SMO are shown in (FIG. 6c) and cell lines less sensitive to LFPRLR SMO at 73 nM are shown in (FIG. 6d). (FIG. 6e) Signaling of PRL through the LF/IFPRLR induces the expression of MYC and BCL2 in overt human DLBCL and B-ALL, thereby promoting malignant B-cell growth and survival. LFPRLR SMO thus represents a promising candidate to treat these malignancies.

FIG. 7: Gating strategy for flow cytometry analysis of mouse splenic WBCs. From the lymphocyte cluster; singlets were gated followed by selection of live (Ghost-UV450⁻) populations. B cells, T cells, and non-B non-T cells were then gated on CD19⁺, CD3⁺, and CD19⁻ CD3⁻, respectively. T cells were gated on CD8⁺, CD4⁺, and CD4⁻CD8⁻ T cells. pDCs and cDCs were gated on CD11c and PDCA1 after gating out NKp46⁺ NK cells from the non-B non-T fraction.

FIG. 8: LFPRLR knockdown did not induce significant changes in frequencies or numbers of T cell subsets in SLE-prone mice. Quantitation of numbers and representative flow cytometry plots of CD4⁺, CD8⁺, and CD4⁻CD8⁻ T cells of MRL-lpr SLE prone mice treated with control SMO (n=14) or LFPRLR SMO to knockdown LFPRLR (n=14). Graphs show median±interquartile range. Exact p-values were calculated using the Mann-Whitney U test. ns=non-significant.

FIGS. 9A-9D: Gating strategy for flow cytometry by intracellular staining. From the lymphocyte cluster; singlets were gated followed by selection of live (Ghost-UV450⁻) populations. (FIG. 9a) Total CD138⁺ plasma cells, CD138⁺B220⁺ plasmablasts, and CD138⁺B220⁻ LLPC were then measured by gating on Blimp1⁺ cells. (FIG. 9b-9c) Total CD19⁺ B cells were then gated and BCL2 (FIG. 9b) or AID (FIG. 9c) or TCL1 (FIG. 9d) was measured within these cells. Gates for all markers were set based on fluorescence minus one controls (FMO).

FIGS. 10A-10B: Gating strategy for flow cytometry analysis and magnetic sorting of mouse splenic B cells. (FIG. 10a) From the lymphocyte cluster; singlets were gated followed by selection of live (Ghost-UV450⁻) populations. B cells were then gated on CD19⁺. (FIG. 10b) Flow cytometry verification of splenic B cells sorted out magnetically using CD19 microbeads from one representative 14-week-old MRL-lpr SLE-prone mouse treated with either control SMO (n=14) or LFPRLR SMO (n=14) for 8 weeks.

FIGS. 11A-11B: LFPRLR knockdown did not induce significant changes in numbers of T cell and DC subsets in DLBCL-prone mice. (FIG. 11a,b) Quantitation of numbers and representative flow cytometry plots of total, CD4⁺, CD8⁺, and CD4⁻CD8⁻ T cells (FIG. 11a) and CD11c⁺PDCA1⁺ pDCs and CD11c⁺PDCA1⁻ cDCs (FIG. 11b) in TCL1-tg DLBCL-prone mice treated with control SMO (n=8) or LFPRLR SMO to knockdown LFPRLR (n=8). Graphs show median±interquartile range. Exact p-values were calculated using the Mann-Whitney U test. ns=non-significant.

FIG. 12: Knockdown of LFPRLR in DLBCL-prone mice does not impact cycling of splenic B cells. Percentages of B cells in sub G0/G1, G0/G1, S, G2/M phases and representative flow cytometry in spleens of TCL1-tg mice treated with control SMO (n=8) or LFPRLR SMO (n=8). Graphs show median±interquartile range. Exact p-values were calculated using the Mann-Whitney U test. ns=non-significant.

FIG. 13: Knockdown of LFPRLR reduced BCL2 expression but does not alter MYC expression in total splenic WBCs of DLBCL-prone mice. Comparison of total splenocyte transcript levels of MYC (left) and BCL2 (right) by qPCR in TCL1-tg mice treated with control SMO (n=8) or LFPRLR SMO (n=8). Graphs show median±interquartile range. Each dot in qPCR analyses of transcripts corresponds to the mean expression of that gene in one mouse calculated from 3 technical replicates. Exact p-values were calculated using the Mann-Whitney U test. ns=non-significant

FIG. 14: Total PRLR expression in diagnosis and treated DLBCL patients does not predict clinical outcome. Comparison of overall survival probabilities of patients with B cell lymphomas from 3 datasets (GSE4475, GSE10846, and E-TABM-346) divided into two groups as PRLR^{High} and PRLR^{Low} based on the median expression of total PRLR mRNA. GSE4475 includes 123 DLBCL and 36 BL patients at diagnosis. GSE10846 includes samples from 414 DLBCL patients collected after treatment with Rituximab-CHOP or CHOP and E-TABM-346 includes samples from 53 DLBCL patients collected after treatment with Rituximab-CHOP or CHOP. P values were measured by Log-rank (Mantel-Cox) test. ns=not significant.

FIG. 15: Normal B cells always express both SFPRLR and LF/IF PRLR but 50% of BCL2/MYC-driven B-ALL express only LF/IF PRLR. Comparison of expression of ratio of LF+IF: total PRLR between normal B cells and patients with BCL2/MYC-driven B-ALL from the St. Jude cohort EGAS00001003266. Graph shows median±interquartile range. Exact p-value was calculated using the Mann-Whitney U test. ns=non-significant.

FIGS. 16A-16D: Gating strategy for flow cytometry analysis and magnetic sorting of human peripheral blood B, T and NK cells. (FIG. 16a) From the lymphocyte cluster; singlets were gated followed by selection of live (Ghost-UV450⁻) populations. B cells were then gated on CD3⁻ CD19⁺, T cells were gated on CD3⁺ CD19⁻ and NK cells were then gated on CD3⁻CD56⁺. (FIG. 16b-d) Verification of post-sort purity of magnetically sorted B (FIG. 16b), T (FIG. 16c) and NK (FIG. 16d) cells from PBMC of healthy donors by flow cytometry.

FIG. 17: Full scan of blot shown in FIG. 5c,d. Blot was developed in ChemiDoc MP imaging system (BioRad).

FIG. 18: List of qPCR primers. Figure discloses SEQ ID NOS 14-21, 20, and 22-42, respectively, in order of appearance.

FIG. 19: An exemplary vivo-morpholino oligonucleotide having the structure of formula I.

FIGS. 20A-20B. PRLR isoforms detected in certain mouse and human B cells. qPCR quantification identified LF and SF3 PRLR isoforms in certain mouse leukocytes (FIG. 20A), and LF/IF, SF1a, and SF1b isoforms in certain normal human B cells (FIG. 20B). LF/IF and SFs have identical extracellular and transmembrane domains but differ in their intracellular domains required for pro-proliferative, anti-apoptotic signaling. S1 and S2 are extracellular domains. STAT-binding exon 10 is an intracellular domain.

FIGS. 21A-21B. Expression of total and LFPRLR is elevated in leukocytes of SLE-prone mice and patients with SLE. (FIG. 21A) Fold change relative to one-month-old mice in the expression of total PRLR mRNA (left) and PRLR-normalized exon 10 reads representing LFPRLR (right) by RNA sequencing in splenic leukocytes of NZB/W mice during SLE progression (n=3) and in healthy control mice (n=2) (GSE186367). (FIG. 21B) Comparison of total PRLR mRNA (left) and normalized exon 10 reads representing LFPRLR (right) in PBMCs of healthy donors (n=10) and patients with SLE (n=20) (GSE211700). P-values: Mann-Whitney U test.

FIG. 22. LFPRLR knockdown in SLE-prone mice reduces STAT3 activation in splenic B cells. Quantitation of median fluorescence intensity (MFI) of phosphorylated (p)-STAT3 by flow cytometry in unstimulated (unfilled circles) and IL-6 stimulated (filled circles) splenic B cells of 14-week-old MRL-lpr SLE-prone mice treated with control SMO (n=5) or LFPRLR SMO (n=5). P-values were calculated by unpaired t-test. Gates were set using fluorescence minus one (FMO) controls.

FIG. 23. Reduction in splenic follicular (germinal center) B cells after LFPRLR knockdown in SLE-prone mice. Quantification (left) and representative flow cytometry plots (right) showing changes in FO and MZ splenic B cells in 14-week-old SLE-prone MRL-lpr mice without (control SMO, n=5) or after LFPRLR knockdown (LFPRLR SMO, n=5). P-values were calculated by Mann-Whitney U test.

FIG. 24. Expression of PRLR is highest in splenic plasma cells of SLE-prone mice. Representative uniform manifold approximation and projection plots (UMAP) depicting single-cell transcriptomes from splenic leukocytes of MRL-lpr SLE-prone mice (n=3) by 5′-scRNAseq. Each dot represents a single cell. Unsupervised clustering of immune subsets was performed by Seurat and ImmGen. PRLR mRNA is most highly expressed in CD138⁺ XBP1⁺ IRF4⁺ BLIMP1⁺ plasma cells.

FIG. 25. Plasma cells of SLE-prone mice predominantly express the LFPRLR. Representative uniform manifold approximation and projection plot (UMAP) depicting single-cell transcriptomes from splenic leukocytes of MRL-lpr SLE-prone mouse by 3′-scRNAseq. Each dot represents a single cell. Unsupervised clustering of immune subsets was performed by Seurat and ImmGen. LFPRLR and SFPRLR are most highly expressed in CD138⁺ XBP1⁺ IRF4⁺ BLIMP1⁺ plasma cells.

FIG. 26. Normal human plasma cells are insensitive to LFPRLR knockdown. Cell viability assessed using MTS assay after treatment with control or LFPRLR SMO for 48 h in one representative of six human healthy donor plasma cell subsets differentiated from their PBMC B cells using CD40L and IL-21. For plasma cell production protocol, see FIG. 28B.

FIG. 27. Normal human proliferating/activated B cells are insensitive to LFPRLR knockdown. Cell viability assessed using MTS assay after treatment with control SMO or LFPRLR SMO for 48 h in one representative of six human healthy B cells expanded from their PBMC B cells using CD40L and IL-4.

FIGS. 28A-28B. Successful expansion of human B cells and plasma cells from PBMCs of healthy donors. (FIG. 28A) Magnetically sorted B cells from healthy PBMCs (day 0) are expanded in the presence of CD40L and IL-4 to produce activated B cells and a small number of plasma cells (day 14). On day 14, IL-4 is replaced with IL-21 to further expand the plasma cell subsets until day 21. (FIG. 28B) Flow cytometry verification of B cell expansion and activation (day 14) and enhanced plasma cell production (day 21). Data from six healthy donors is shown. IgG high class-switched plasma cells steadily increase in all but one case. Non-class switched (IgD) plasma cells are reduced on day 21. Plasma cell markers CD27, CD138, CD38 steadily increase. HLA-DR, CD19, BCMA increase with B-cell activation/expansion (day 14) but reduce with plasma cell production (day 21).

FIG. 29. Anti-dsDNA autoantibody production trended towards a reduction after LFPRLR knockdown. Fold change/increase in anti-dsDNA antibodies (day 56: day 9) in mice treated with either control SMO (n=7) or LFPRLR SMO (n=7). Exact p-values were calculated using the Mann-Whitney U test.

FIG. 30. Expression of LF and IFPRLR in malignant human B cells. LFPRLR (left) and IFPRLR (right) mRNA levels in malignant human B-cell lines (SU-DHL-6, Karpas-422, VAL and OCI-LY18) treated with their IC50 concentration of control SMO or LFPRLR SMO for 48 h. Each qPCR sample was run in 3 technical replicates. For bars where <3 dots are shown, the remaining values are zero and could not be plotted on log scale. UBB was used as the house keeping gene in qPCR.

FIG. 31. Changes in STAT5 activation and level after LFPRLR knockdown in malignant B cells. Immunoblotting showing reduction in global STAT5 levels in three out of four malignant B cell lines treated at their IC50 concentrations of LFPRLR SMO for 12 hours. beta-actin was used as the loading control in immunoblotting.

FIG. 32. Immunoblotting showing reduction in active phosphorylated STAT3 and global STAT3 levels in three out of four malignant B cell lines treated at their IC50 concentration of LFPRLR SMO for 3, 8, 12 and 48 h. β-actin was used as the loading control in immunoblotting. P values for survival curves were measured by log-rank (Mantel-Cox) test. For all other experiments, p-values were calculated by unpaired t test from 3 independent experiments/biological replicates (±SEM), unless otherwise indicated. qPCR of each biological sample was conducted in three technical replicates. UBB was used as the house-keeping gene in qPCR. For all flow cytometry-based measurements of cell death, the IC50 concentration of the Karpas-422 line was used (73 nM). FPKM=fragments per kilobase of transcript per million mapped reads.

FIGS. 33A-33D. Knockdown of LFPRLR reduces progression of overt human DLBCL in vivo. FIG. 33a. Experimental design for measuring the in vivo efficacy of LFPRLR SMO in reducing progression of overt human DLBCL in malignant B-cell line derived xenograft (CDX) models. FIG. 33b. qPCR validation of LFPRLR knock down in tumors isolated from euthanized transplant recipient mice treated with control SMO (n=6 for SU-DHL-6 and n=5 for OCI-LY18, red) or LFPRLR SMO (n=4 for SU-DHL-6 and n=6 for OCI-LY18, blue) on day 15. Each dot in qPCR analyses of transcripts corresponds to the mean expression of that gene in tumors from one CDX recipient mouse calculated from 3 technical replicates. UBB was used as the house-keeping gene in qPCR. FIG. 33c, d. Change in tumor volume and tumor weight on the day of euthanasia (day 15) in NSG mice that received subcutaneous injections of 7×10⁶ malignant human B cells [SU-DHL-6 (FIG. 33c) and OCI-LY18 (FIG. 33d)] and were treated with control SMO (n=6 for SU-DHL-6 and n=5 for OCI-LY18, red) or LFPRLR SMO (n=6 for SU-DHL-6 and n=6 for OCI-LY18, blue). Tumor volume panels in (FIG. 33c, d) show mean±SEM. All other graphs show median±interquartile range. Exact p-values were calculated using the Mann-Whitney U test. ns non-significant, *0.01<p<0.05, **0.001<p<0.01.

FIGS. 34A-34B. FIG. 34a PRL secretion level by ELISA and b viable cell number after treatment with rabbit anti-PRL or rabbit normal serum, assessed by MTS assay in malignant human B-cell lines. P-values by unpaired t test at different concentrations of anti-PRL or normal serum in FIG. 34b: SU-DHL-6 (P=0.0009 in 1:1600 and P<0.0001 in 1:800, 1:400, 1:200, 1:100 and 1:50), Karpas-422 (P=0.0283 in 1:1600, P=0.0016 in 1:800, P=0.0004 in 1:200 and P<0.0001 in 1:400, 1:100 and 1:50), VAL (P=0.0066 in 1:1600, P=0.0178 in 1:100 and P=0.0002 in 1:50) and OCI-LY18 (P=0.0253 in 1:1600, P=0.0007 in 1:800, P=0.0051 in 1:100, P=0.0007 in 1:50). P-values were calculated by unpaired t test (+SD). P values for 1:50 are indicated in the figure. ns non-significant.

FIG. 35. With the experimental design of FIG. 4, TCL1 was also assessed (n=6).

FIG. 36. Neutralization of PRL secreted by malignant human B cells reduces their viability. DAPI⁺ dead cells were assessed by flow cytometry in malignant human B-cell lines after treatment with 1:50 dilution of normal rabbit serum or rabbit anti-human PRL for 48 h.

### DETAILED DESCRIPTION

Prolactin (PRL) is a hormone originally known for its secretion from the pituitary gland and its ability to promote lactation in mammals. However, prolactin also plays other less well elucidated roles in regulating other systems. Prolactin receptor (PRLR) pre-mRNA may be alternatively spliced into a variety of mature mRNA forms, including but not limited to a long form, intermediate form, and short forms (e.g., SF1a, SF1b and SF1c in the human). For example, the long form receptor (also called “LF PRLR”) comprises exons 3-10 spliced together in humans and mice. The intermediate form (IF) in humans comprises exons 3-9 plus 2 pieces of exon 10, whereas the short forms of the human receptor (also called “SF PRLR”) are characterized by inclusion of differing proportions of exon 11 spliced to exons 3-9. Human SF1a also has a short 154 nucleotide piece of exon 10. In mice, the short forms link exons 3-9 to either exon 11, 12 or 13. These splice forms share identical extracellular domains but differ in intracellular domains. Hence, distinct intracellular signaling is mediated by the different forms.

As described herein, the long form receptor (LF PRLR) and intermediate form receptor (IF PRLR) mediate signals from prolactin that are pro-cancerous or promote survival/proliferation, for example, via increased levels of oncoprotein and/or anti-apoptotic factors, whereas the short forms of the receptor (SF PRLR) generally mediate signals that are anti-cancerous. Additionally, prolactin exacerbates certain autoimmune disorders, such as SLE, which in turn can progress to certain B cell malignancies, such as DLBCL or B-CLL. As described in Example 1, expression of the long form receptor (LF PRLR) and intermediate form receptor (IF PRLR) can be specifically inhibited using a splice modulating oligonucleotide (SMO), and the downstream effects of prolactin that lead to undesirable B cell survival/proliferation in B cell disorders, such as B malignancies and/or SLE, can be suppressed (see, e.g., Example 1, LF/IF mRNA expression as compared to total PRLR). For example, exacerbation of SLE and the progression into B cell lymphoma (e.g., DLBCL) or B-CLL can be suppressed. Unlike drugs that inhibit secretion of prolactin from the pituitary gland, inhibiting LF/IF PRLR expression blocks the effects of non-pituitary sources of prolactin, such as DLBCL cells. A LF/IF PRLR-specific treatment can also leave intact certain other prolactin receptor isoforms (e.g., SF1a, SF1b and/or SF1c), preserving many of the beneficial aspects of prolactin (see, e.g., Example 1). Finally, SMO treatment targeting LF/IF PRLR is less toxic than current standard of care therapies.

Accordingly, certain embodiments of the invention provide a method of inhibiting the expression of LF/IF PRLR in a B cell, comprising contacting the B cell with a splice modulating oligonucleotide (SMO) as described herein. In certain embodiments, the B cell is contacted in vitro. In certain embodiments, the B cell is contacted in vivo.

As used herein, the term “LF/IF PRLR” refers to long form (LF) PRLR and/or intermediate form (IF) PRLR. In certain embodiments, a cell expresses LF PRLR. In certain embodiments, a cell expresses IF PRLR. In certain embodiments, a cell expresses both LF PRLR and IF PRLR. In certain embodiments, expression of LF PRLR in a cell is inhibited by a SMO. In certain embodiments, expression of IF PRLR in a cell is inhibited by a SMO. In certain embodiments, expression of both LF PRLR and IF PRLR in a cell are inhibited by a SMO.

In certain embodiments, such a method reduces the expression of activation-induced cytidine deaminase (AID), anti-apoptotic factor BCL-2, and/or oncoprotein c-Myc as compared to a control (e.g., as compared to expression levels in the cell prior to contact with the SMO or as compared to levels in a corresponding control cell that was not contacted the SMO). Thus, certain embodiments also provide a method of reducing the expression of AID, BCL2, and/or oncoprotein cMyc in a B cell that expresses LF/IF PRLR comprising contacting the B cell with a splice modulating oligonucleotide (SMO) as described herein. In certain embodiments, the B cell is contacted in vitro. In certain embodiments, the B cell is contacted in vivo.

Certain embodiments of the invention also provide a method of inhibiting prolactin or LF/IF PRLR mediated B cell survival and/or proliferation in a subject in need thereof, comprising administering a SMO to the subject, wherein the SMO inhibits expression of the LF/IF PRLR (e.g., B cell expression of LF/IF PRLR) and/or reduces the relative expression of LF/IF PRLR compared to the expression of one or more SF PRLRs (e.g., relative B cell expression of LF/IF PRLR to one or more SF PRLRs). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

Certain embodiments of the invention provide a method of reducing B cell proliferation and/or number in a subject in need thereof, comprising administering a SMO to the subject, wherein the SMO inhibits expression of LF/IF PRLR (e.g., B cell expression of LF/IF PRLR) and/or reduces the relative expression of LF/IF PRLR compared to the expression of one or more SF PRLRs (e.g., relative B cell expression of LF/IF PRLR to one or more SF PRLRs). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

Certain embodiments also provide a method of treating or preventing a B cell mediated disorder in a subject in need thereof, comprising administering a SMO to the subject, wherein the SMO inhibits the expression of LF/IF PRLR (e.g., inhibiting B cell expression of LF/IF PRLR) and/or reduces the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

In certain embodiments, the method is for treating a B cell mediated disorder. In certain embodiments, the method is for preventing a B cell mediated disorder.

As used herein, the term “B cell mediated disorder” refers to a disease, disorder or condition that is characterized by abnormal B cells. The term “abnormal B cell” refers to a B-lineage cell, a mature or immature B cell, or its progenitor/precursor thereof (e.g., pro-B cells, pre-B cells), a B-lineage lymphoblast (B-lymphoblast), a memory B cell, a plasmablast, or a plasma cell, that is malignant, autoreactive, has a tendency of excessive or uncontrolled proliferation/clonal expansion, and/or has resistance against apoptosis. An abnormal B cell described herein may be either circulating in blood/lymphatic systems, or mobile or resident at immune and nonimmune organs/tissues throughout the body, including but not limited to, lymph nodes, spleen, bone marrow, liver, skin, lymphoid tissues, such as mucosa associated lymphoid tissue and skin associated lymphoid tissue, and other organs/tissues into which malignant B cells may infiltrate or metastasize.

In one embodiment, the B cell mediated disorder is associated with an excessive number/proliferation of B cells (e.g., malignant B cells, or abnormal clonal expansion of certain B cells). In certain embodiments, the disorder is associated with an abnormal B cell receptor (BCR) repertoire characterized by, for example, an excessive amount of autoreactive B cells or an abnormal clonal expansion of certain B cells with a BCR against certain autoantigen(s) and/or foreign antigen(s). In certain embodiments, the B cell mediated disorder is associated with a BCR repertoire characterized by an excessive amount of non-functional IgH sequences and/or an excessive amount of BCRs having a CDR3 region (e.g., CDR3 of heavy chain) with a length greater than about 20 or 25 amino-acids (aa).

In certain embodiments, the subject comprises abnormal B cells that are malignant and/or autoreactive. In certain embodiments, the subject comprises abnormal B cells that are malignant. In certain embodiments, subject comprises abnormal B cells that are autoreactive.

In certain embodiments, subject comprises B cells (e.g., abnormal B cell, such as DLBCL cells) that overexpress prolactin and/or LF/IF PRLR as compared to a control (e.g., from a B cell or B cell population from a healthy person or a person that does not have a B cell mediated disorder). The overexpression may be at the mRNA and/or protein level. In certain embodiments, subject comprises B cells (e.g., DLBCL cells) that overexpress LF/IF PRLR. In certain embodiments, LF/IF PRLR is overexpressed by at least about 10%, at least about 15%, at least about 20%, at least about 25%, at least about 30%, at least about 40%, at least about 50%, at least about 60%, at least about 70%, at least about 80%, at least about 90%, at least about 100%, at least about 200%, at least about 300%, at least about 400%, at least about 500%, or more as compared to a control or relative to one or more SF PRLRs. In certain embodiments, subject comprises B cells (e.g., DLBCL cells) that overexpress prolactin. In certain embodiments, prolactin is overexpressed by at least about 10%, at least about 15%, at least about 20%, at least about 25%, at least about 30%, at least about 40%, at least about 50%, at least about 60%, at least about 70%, at least about 80%, at least about 90%, at least about 100%, at least about 200%, at least about 300%, at least about 400%, at least about 500%, or more as compared to a control. Methods for detecting or measuring the expression of prolactin or PRLR (e.g., LF/IF PRLR and/or SF PRLR) are known in the art (e.g., RT-PCR, ELISA, western blot). For example, an assay described herein may be used.

In certain embodiments, the B cell mediated disorder is a B cell malignancy, such as B cell lymphoma, B cell chronic lymphocytic leukemia (B-CLL), or B cell acute lymphoblastic leukemia (B-ALL). In certain embodiments, the B cell malignancy is diffuse large B cell lymphoma (DLBCL) or Burkitt's lymphoma (BL). In certain embodiments, the B cell lymphoma is a non-Hodgkin's lymphoma (NHL). In certain embodiments, the NHL is diffuse large B cell lymphoma (DLBCL). In certain embodiments, the NHL is Burkitt's Lymphoma (BL). In certain embodiments, the NHL is follicular lymphoma. In certain embodiments, the B cell mediated disorder is the autoimmune disease systemic lupus erythematosus (SLE; lupus) which may progress to DLBCL. Other autoimmune B cell disorders with elevated PRL that predispose to B cell malignancies such as DLBCL include rheumatoid arthritis (RA) and Sjögren's syndrome (SS).

In certain embodiments, the B cell mediated disorder is a disorder involving a pre-leukemic B cell clone which may progress to B-ALL.

In certain embodiments, the B cell mediated disorder is a disorder that leads to a B cell malignancy. For example, as described herein, subjects having certain autoimmune disorders, such as SLE, have an increased likelihood of developing a B cell malignancy (e.g., DLBCL or B-CLL). Accordingly, in certain embodiments, the B cell mediated disorder is a B cell malignancy (e.g., DLBCL or B-CLL) that progressed from an autoimmune disorder (e.g., SLE). In certain embodiments, the B cell malignancy is a non-Hodgkin's lymphoma (e.g., DLBCL) that progressed from SLE. In certain embodiments, the B cell malignancy is B cell acute lymphoblastic leukemia (e.g., B-ALL) that progressed from a pre-leukemic B cell clone. In certain embodiments, the B cell mediated disorder is an indolent B cell disorder. In certain embodiments, the B cell malignancy is indolent B cell lymphoma.

Thus, certain embodiments also provide a method of reducing a subject's likelihood of developing a B cell mediated disorder, the method comprising administering a SMO to the subject, wherein the SMO inhibits expression of LF/IF PRLR (e.g., B cell expression) and/or reduces the relative expression of LF/IF PRLR compared to the expression of one or more SF PRLRs (e.g., relative B cell expression). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

Certain embodiments also provide a method of reducing a subject's likelihood of developing a B cell malignancy (e.g., likelihood of B cell lymphomagenesis or B cell leukemogenesis), wherein the subject has an autoimmune disease or has premalignant B cell clones, the method comprising administering a SMO to the subject, wherein the SMO inhibits expression of LF/IF PRLR (e.g., B cell expression) and/or reduces the relative expression of LF/IF PRLR compared to the expression of one or more SF PRLRs (e.g., relative B cell expression). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

In certain embodiments, the method is for preventing a subject from developing a B cell mediated disorder, such as a B cell malignancy. In certain embodiments, the method is for preventing SLE-associated B cell lymphomagenesis.

In certain embodiments, the autoimmune disease is SLE, RA, or SS.

In certain embodiments, the B cell mediated disorder is a B cell malignancy, such as B cell lymphoma (e.g., DLBCL), B-CLL, or B-ALL.

In certain embodiments, the B cell mediated disorder is B cell lymphocytosis (e.g., monoclonal B cell lymphocytosis).

In certain embodiments, a pre-treatment subject had, or was determined to have, increased LF/IF PRLR expression (e.g., increased LF/IF PRLR B cell expression) as compared to a control. In certain embodiments, a pre-treatment subject had, or was determined to have, increased prolactin expression (e.g., increased expression of prolactin in B cells or increased concentration of prolactin in a blood sample) as compared to a control.

In certain embodiments, administration of the SMO inhibits the expression (e.g., B cell expression) of LF/IF PRLR. In certain embodiments, administration of the SMO reduces the relative expression (e.g., relative B cell expression) of LF/IF PRLR compared to one or more SF PRLRs expression (e.g., SF1b and/or SF1c expression; or SF1a and/or SF1b expression). In certain embodiments, administration of the SMO reduces the relative expression of LF/IF PRLR over total PRLR expression (e.g., LF+IF+SF).

As described herein, certain B cells or clones (e.g., certain normal B cells) may produce no PRL and express both LF/IF and SF PRLRs, but other B cells or clones (e.g., certain abnormal B-lymphoblast from B-ALL patient) may produce PRL and express LF PRLR but not SF PRLRs. In certain embodiments, B cells or clones (e.g., certain abnormal B-lymphoblast from B-ALL patient) may express LF and/or IF PRLR but not SF PRLR. Therefore, as used herein, the term “inhibit” may refer to reducing LF/IF PRLR expression as compared to a control (e.g., as compared to levels in the subject prior to administration or as compared to levels in a control subject having a corresponding B cell mediated disorder that was not administered the SMO), as well as prophylactically preventing a relative increase in LF/IF PRLR expression as compared to a control.

In certain embodiments, administration of a SMO described herein inhibits expression of LF/IF PRLR. In certain embodiments, administration of a SMO described herein inhibits B cell expression of LF/IF PRLR. In certain embodiments, the SMO inhibits the expression of LF/IF PRLR by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100%, at least about 200%, at least about 300%, or more (e.g., as compared to a control value, such as compared to expression levels in the subject prior to treatment with the SMO or as compared to levels in a control subject having a corresponding B cell mediated disorder that was not administered the SMO). In certain embodiments, administration of the SMO reduces the relative expression (e.g., relative B cell expression) of LF/IF PRLR compared to total PRLR expression by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100%, at least about 200%, at least about 300%, or more (e.g., as compared to a control value, such as compared to expression levels in the subject prior to treatment with the SMO or as compared to levels in a control subject having a corresponding B cell mediated disorder that was not administered the SMO). In certain embodiments, the LF/IF PRLR expression levels after administration are comparable to levels in a heathy subject or a subject that does not have a B cell mediated disorder. In certain embodiments, a SMO as described herein is administered prophylactically. In such embodiments, administration may prevent LF/IF PRLR expression or onset of expression may be slowed. Methods for measuring the expression of LF/IF PRLR and total PRLR are known in the art. For example, in certain embodiments, an assay (e.g., RT-PCR, western blot, or RNA sequencing) described herein may be used.

In certain embodiments, administration of a SMO described herein inhibits expression (e.g., B cell expression) of the LF PRLR. In certain embodiments, administration of a SMO described herein inhibits expression (e.g., B cell expression) of the IF PRLR.

In certain embodiments, administration of the SMO reduces the expression (e.g., B cell expression) of activation-induced cytidine deaminase (AID), anti-apoptotic factor BCL-2, and/or oncoprotein c-Myc as compared to a control (e.g., as compared to expression levels in the subject prior to treatment with the SMO or as compared to levels in a control subject having a corresponding B cell mediated disorder that was not administered the SMO). In certain embodiments, the administration reduces the expression of AID (e.g., B cell expression of AID is reduced). In certain embodiments, the administration reduces the expression of BCL-2 (e.g., B cell expression of BCL-2 is reduced). In certain embodiments, the administration reduces the expression of c-Myc (e.g., B cell expression of c-Myc, such as B-lymphoblast expression of c-Myc, is reduced). In certain embodiments, the administration reduces the expression of c-Myc and BCL-2. In certain embodiments, the administration reduces the expression of AID. The reduced expression may be at mRNA level and/or protein level. In certain embodiments, the administration independently reduces the expression of AID, Bcl-2, and/or c-Myc by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, at least about 100%, at least about 200%, at least about 300%, at least about 400%, at least about 500%, or more, as compared to a control, e.g., such as compared to expression levels in the subject prior to treatment with the SMO or to a control subject that was not administered the SMO (e.g., one embodiment of SMO for LF/IF PRLR inhibition described herein is also referred to as Antimaia (see, Example 1, wherein Antimaia is also referred to as LFPRLR SMO)).

In certain embodiments, the administration of the SMO reduces the viability of abnormal B cells as compared to a control. In certain embodiments, the administration of the SMO reduces excessive proliferation or clonal expansion of abnormal B cells as compared to a control. In certain embodiments, the administration of the SMO reduces abnormal B cells' resistance against apoptosis or cell-killing as compared to a control. In certain embodiments, the administration of the SMO increases the proportion of dormant B cells at cell cycle phase G0 and/or G1 phase as compared to a control. In certain embodiments, the administration of the SMO sensitizes abnormal B cells to an antibody or immune cell-based immunotherapy. In certain embodiments, the administration of the SMO sensitizes abnormal B cells to immune cell mediated cytotoxicity (e.g., Natural Killer (NK) cell mediated cytotoxicity). In certain embodiments, the administration of the SMO sensitizes abnormal B cells to NK cell mediated cytotoxicity, wherein the expression of c-Myc in NK cells is not reduced. Methods for measuring cell viability, cell cycle and/or proliferation are known in the art. For example, in certain embodiments, an assay described herein (e.g., viability, cell cycle, proliferation, or cytotoxicity assay) may be used.

In certain embodiments, the administration of the SMO reduces white blood cell (WBC) numbers or count in the subject (e.g., in a sample, such as spleen) as compared to a control. In certain embodiments, the administration of the SMO reduces B cell numbers or count in the subject (e.g., in a sample, such as spleen). In certain embodiments, the administration of the SMO reduces total B cell numbers or count in the subject (e.g., in a sample, such as spleen). In certain embodiments, the administration of the SMO reduces plasma cell (e.g., Blimp1⁺CD138⁺B220⁻ long-lived plasma cells (LLPCs) or Blimp1⁺CD138⁺B220⁺ short-lived plasmablasts) numbers or count in the subject (e.g., in a sample, such as spleen). In certain embodiments, the administration of the SMO reduces B-lymphoblast numbers or count in the subject (e.g., in a sample, such as spleen). In certain embodiments, the administration of the SMO reduces malignant and/or autoreactive B cell numbers or count in the subject (e.g., in a sample, such as spleen). In certain embodiments, the administration of the SMO reduces the dissemination of the pre-malignant bone marrow B cells into the periphery (e.g., in a sample, such as spleen). In certain embodiments, the administration of the SMO reduces dendritic cell (e.g., plasmacytoid (pDC) and/or conventional DC (cDC)) numbers or count in the subject (e.g., in a sample, such as spleen). In certain embodiments, the administration of the SMO reduces dendritic cell (e.g., pDC and/or cDC) numbers or count in a subject with an autoimmune disease (e.g, SLE). Methods for measuring WBC, DC cell, or B cell number are known in the art. For example, in certain embodiments, an assay described herein may be used. Any suitable cytometry, histology, or imaging technology suitable for directly or indirectly counting B cells may be used. In certain embodiments, flow cytometry is used in which one or more B cell marker (e.g., CD19, CD20, CD138, Blimp1, or B220) may be labeled. In certain embodiments, CD19 and/or CD20 marker is used for B cells (e.g., CD19⁺ B cells, CD20⁺ B cells, or CD19⁺/CD20⁺ B cells). In certain embodiments, imaging technology such as positron emission tomography (PET) is used. In certain embodiments, the administration of the SMO reduces B cell numbers or a B cell lymphoma tumor volume by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, at least about 100%, at least about 200%, at least about 300%, at least about 400%, at least about 500%, or more as compared to a control, e.g., the subject prior to treatment with the SMO or a control subject that was not administered the SMO (e.g., Antimaia/LFPRLR SMO as described in Example 1).

In certain embodiments, the administration of the SMO modifies the subject's B cell receptor (BCR) repertoire. For example, in certain embodiments, the percentage of non-functional IgH and/or the percentage of BCRs with long CDR3 regions (e.g., long heavy chain CDR3 regions that are >20 amino acid in length) is reduced in the subject. In certain embodiments, the percentage of BCRs with long CDR3 regions (e.g., >20aa, 25aa, 30aa, 35aa, or 40aa, such as 35-42aa in length) is reduced in the subject, e.g., as compared to a control such as the subject prior to treatment with the SMO or a control subject that was not administered the SMO. In certain embodiments, the percentage of non-functional IgH and/or the percentage of BCRs with long CDR3 regions (e.g., >20aa, 25aa, 30aa, 35aa, or 40aa, such as 35-42aa in length) is reduced by at least about 0.2%, at least about 0.5%, at least about 1%, at least about 2%, at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, at least about 100%, at least about 200%, at least about 300%, at least about 400%, at least about 500%, or more as compared to a control, e.g., the subject prior to treatment with the SMO or a control subject that was not administered the SMO (e.g., Antimaia/LFPRLR SMO as described in Example 1).

In certain embodiments, the administration of the SMO reduces B cell somatic hypermutation of V_{H} and/or CDR3 domains. Without wanting to be bound by theory, such a result is consistent with reduced AID expression and activity.

Certain embodiments also provide a method of reducing the expression of AID, BCL2, and/or oncoprotein cMyc in a subject in need thereof (e.g., a subject who has cancer) comprising administering a SMO as described herein to the subject, wherein the SMO is capable of inhibiting the expression of LF/IF PRLR (e.g., inhibiting B cell expression of LF/IF PRLR) and/or is capable of reducing the relative expression of LF/IF PRLR compared to the expression of SF PRLR (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1). For example, the proportion of LF/IF PRLR as compared to total PRLR is reduced and the proportion of SF PRLR(s) as compared to total PRLR is increased.

In certain embodiments, the method reduces the expression of AID (e.g., as compared to a control). In certain embodiments, the method reduces the expression of BCL2 (e.g., as compared to a control). In certain embodiments, the method reduces the expression of cMyc (e.g., as compared to a control).

In certain embodiments, the method may further comprise administering one or more additional therapeutic agents as described herein.

Certain embodiments also provide a SMO as described herein for reducing the expression of AID, BCL2, and/or oncoprotein cMyc in a subject in need thereof, wherein the SMO is capable of inhibiting the expression of LF/IF PRLR (e.g., inhibiting B cell expression of LF/IF PRLR) and/or is capable of reducing the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1).

Certain embodiments provide the use of a SMO as described herein for the preparation of a medicament for reducing the expression of AID, BCL2, and/or oncoprotein cMyc in a subject in need thereof, wherein the SMO is capable of inhibiting the expression of LF/IF PRLR (e.g., inhibiting B cell expression of LF/IF PRLR) and/or is capable of reducing the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1).

In certain embodiments, such a SMO may be for use/used in combination with one or more additional therapeutic agents described herein.

In certain embodiments, such a SMO may be for use/used as a single therapeutic agent.

In certain embodiments, such a SMO may be for use/used as a single therapeutic agent, or optionally in combination with one or more therapeutic agent(s), to treat a B cell malignancy (e.g., DLBCL, follicular lymphoma) in a subject.

In certain embodiments, such a SMO may be for use/used as a single therapeutic agent(s), or optionally in combination with one or more therapeutic agent, to treat an autoimmune disease in a subject. In certain embodiments, the subject has SLE (e.g., a SLE patient with or without a B cell malignancy).

In certain embodiments, PMBCs, or B cells (e.g., plasma cells and/or their precursor B cells), overexpress LF/IF PRLR, and/or total PRLR in a subject (e.g., a subject having SLE), as compared to a control.

For example, in certain embodiments, pathogenic, autoreactive B cells (e.g., pathogenic, autoreactive plasma cells and/or their precursor B cells) in a subject (e.g., a subject having SLE) may have at least 15%, 20%, 30%, 40%, 50%, 60%, 70%, 80%, 90%, 100%, 200%, 300% or more, higher LF/IF PRLR expression level as compared to the expression level of the normal, non-autoreactive B cells (e.g., plasma cells and/or their precursor B cells) in the same subject, or as compared to the expression level in PBMCs or B cells or counterpart subset(s) in a healthy subject or population. Accordingly, the level of expression of LF/IF PRLR may distinguish between pathogenic, autoreactive B cells and normal, non-autoreactive B cells. Thus, pathogenic, autoreactive B cells (e.g., plasma cells and/or their precursor B cells) may be selectively inhibited or eradicated by a SMO described herein, while normal, non-autoreactive B cells may be spared. In certain embodiments, plasma cells (e.g., CD138⁺ plasma cells, Blimp1⁺CD138⁺ plasma cells, or Blimp1⁺CD138⁺XBP1⁺ IRF4⁺ plasma cells) overexpress LF/IF PRLR, and/or total PRLR in the subject.

In certain embodiments, the number of B cells (e.g., pathogenic, autoreactive B cell) is reduced (e.g., in a sample such as blood, spleen, and/or bone marrow) in the subject (e.g., a subject having SLE) after the administration of an agent described herein (e.g., a SMO). For example, the number of B cells (e.g., plasma cells, and/or their precursor B cells) in a post-administration sample is reduced as compared to a control such as the number prior to the administration of an agent (e.g., SMO), or the number of a patient (e.g., a patient with autoimmune disease such as SLE) or patient population. In certain embodiments, the post-administration sample is a blood sample. In certain embodiments, the post-administration sample is a spleen marrow sample. In certain embodiments, the post-administration sample is a bone marrow sample. In certain embodiments, the post-administration sample is a biopsy sample. In certain embodiments, the number of B cells (e.g., pathogenic, autoreactive B cell) is reduced by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, at least about 100%, at least about 200%, at least about 300%, at least about 400%, at least about 500%, or more as compared to a control, e.g., the subject prior to treatment with the SMO or a control subject that was not administered the therapeutic SMO (e.g., Antimaia/LFPRLR SMO as described in Example 1).

In certain embodiments, the number of plasma cells (e.g., pathogenic, autoreactive plasma cell) is reduced. In certain embodiments, plasma cells may be detected by one or more markers selected from the group consisting of CD138⁺, Blimp1⁺, XBP1⁺, IRF4⁺, CD27⁺, and CD38⁺. For example, in certain embodiments, the plasma cells are CD138⁺ plasma cells, CD38⁺ plasma cells, CD27⁺ plasma cells, Blimp1⁺CD138⁺ plasma cells, or CD138⁺ XBP1⁺ IRF4⁺ BLIMP1⁺ plasma cells. In certain embodiments, plasma cells are CD19^{low} plasma cells. In certain embodiments, plasma cells are CD20^{-/low} plasma cells. In certain embodiments, the number of plasmablast cells is reduced. In certain embodiments, the number of Blimp1⁺CD138⁺B220⁺ plasmablast cells is reduced. In certain embodiments, the number of CD19⁺CD38^{hi}CD27^{hi}CD269⁺MHCII⁺ plasmablast cells is reduced. In certain embodiments, the number of long-lived plasma cells (LLPC) is reduced. In certain embodiments, the number of Blimp1⁺CD138⁺B220⁻ LLPC is reduced. In certain embodiments, the number of Blimp1⁺CD38^{hi}CD27⁺CD269⁺MHCII⁻ long-lived plasma cells (LLPC) is reduced. In certain embodiments, the number of CD138⁺Blimp1⁺CD38^{hi}CD27⁺CD269⁺MHCII⁻ long-lived plasma cells (LLPC) is reduced. In certain embodiments, the plasma cells secrete IgG, or IgA, or IgM. In certain embodiments, the plasma cells secrete IgE.

In certain embodiments, the number of precursor B cells (e.g., pathogenic, autoreactive precursor B cell) that are capable of giving rise to plasma cells (e.g., pathogenic, autoreactive plasma cells) is reduced (e.g., in a sample such as blood, spleen, and/or bone marrow) in the subject (e.g., a subject having SLE). In certain embodiments, the precursor B cells may be detected by one or more markers selected from the group consisting of CD19⁺, CD20⁺, CD22⁺, and B220⁺. In certain embodiments, the precursor B cells are CD19⁺CD20⁺ precursor B cells. Additionally, follicular (FO) B cells may give rise to potentially autoreactive, class-switched plasma cells, thus, in certain embodiments, the number of follicular (FO) B cells is reduced. In certain embodiments, the FO B cells are CD19⁺ CD21⁺ CD23⁺ CD93⁻ FO B cells. In certain embodiments, the FO B cells are CD19⁺CD20⁺CD21⁺CD10⁻ FO B cells. In certain embodiments, the ratio of the number of follicular (FO) B cells over the number of marginal zone (MZ) B cells is reduced. In certain embodiments, the MZ B cells are CD19⁺ CD21⁺ CD23⁻ CD93⁻ MZ B cells. In certain embodiments, the MZ B cells are CD1c⁺CD19⁺CD20⁺CD21^{hi}CD27^{var} MZ B cells. In certain embodiments, the expression level of AID is reduced in precursor B cells as described herein, such as a germinal center B cell (e.g., CD10⁺CD19⁺CD20⁺CD23⁺CD27⁺CD38^{hi}CD269⁺BCMA⁺CD24^{-/low} germinal center B cell).

In certain embodiments, the number of B cells, such as plasma B cells or their precursor B cells, having long BCR and/or Ig CDR3 region (CDR3 having >20 aa in length) is reduced.

In certain embodiments, both of 1) the number of plasma cells (e.g., pathogenic, autoreactive plasma cell), and 2) the number of their precursor B cells (e.g., pathogenic, autoreactive precursor B cell) that are capable of giving rise to plasma cells are reduced (e.g., in a sample such as blood, spleen, and/or bone marrow) in the subject (e.g., a subject having SLE) by a SMO described herein (e.g., SMO as a sole therapeutic agent, or optionally in combination with other therapeutic agent(s)).

In certain embodiments, the number of normal, non-autoreactive B cells is not reduced (e.g., in a sample such as blood, spleen, and/or bone marrow) in the subject (e.g., a subject having SLE). In certain embodiments, the number of normal, non-autoreactive plasma cells is not reduced in the subject. In certain embodiments, the number of normal, non-autoreactive precursor B cells is not reduced in the subject.

In certain embodiments, the number of normal, non-autoreactive B cells is reduced by less than 30%, 20%, 15%, 10%, or 5% by the administered SMO.

As used herein, the term “pathogenic, autoreactive” B cells in a subject (e.g., a subject having autoimmune disorder such as SLE) refers to B cells (e.g., plasma cells) that are reactive to self-antigens. For example, pathogenic, autoreactive B cells may have autoreactive BCRs and/or produce autoantibodies (e.g., anti-DNA or anti-histone autoantibodies). As used herein, the term “normal, non-autoreactive” B cells in a subject refers to B cells (e.g., plasma cells) that might be reactive to foreign antigen(s) and are not reactive to self-antigens.

As used herein, the term “plasma cell” refers to an antibody-secreting cell, including short-lived plasma cell, and long-lived plasma cell. As used herein, the term “plasmablast” refers to a plasma cell that is still capable of proliferating. In certain embodiments, the short-lived plasma cell has a lifespan of less than a month, two weeks, or one week, such as about 3-7 or 3-5 days. As used herein, the term “long-lived plasma cell” or “LLPC” refers to a plasma cell that lives for month(s), year(s) or decade(s). Both short-lived and long-lived plasma cells are terminally differentiated and non-proliferating. In certain embodiments, the LLPC has a life span that is greater than 2, 3, 4, 5, or 6 months. In certain embodiments, the LLPC has a life span that is greater than a year. As used herein, the term “precursor B cell” or “predecessor B cell” refers to a cell committed to the B lineage and capable of giving rise to a plasma cell. In certain embodiments, this precursor B cell may be a mature B cell (e.g., follicular B cell, or an antigen-activated B cell, etc.).

In certain embodiments, the level of autoreactive antibodies is reduced (e.g., in a sample such as blood or serum sample) in the subject (e.g., a subject having SLE). Autoreactive antibodies include but are not limited to, antinuclear antibodies (ANA), such as anti-DNA antibodies, and/or anti-histone antibodies. In certain embodiments, the level of anti-DNA antibodies (e.g., anti-double stranded DNA) is reduced. In certain embodiments, the level of anti-histone antibodies is reduced. Methods of detecting the level of autoreactive antibodies are known in the art (e.g., ELISA and immunofluorescence assay) and also described herein (e.g., see Examples 2-4 below). In certain embodiments, the pathologically elevated level of autoreactive antibodies in the subject (e.g., a subject having SLE) may be reduced to a level that is closer to or similar to a control or reference, e.g., the level of a healthy subject or population. In certain embodiments, the level of autoreactive antibodies is maintained at a level similar to a control or reference, e.g., the level of a healthy subject or population.

In certain embodiments, the level of protein in a urine sample (proteinuria) of the subject (e.g., a subject having SLE) is reduced. For example, a patient subject having SLE may have autoantibodies reactive against self that accumulate in the kidneys or antibodies reactive with self-antigens in kidney, leading to glomerulonephritis, and/or pathologically elevated protein level in urine (proteinuria). Methods of detecting the level of protein in a urine sample are known in the art and also described herein (e.g., see Example 3 below). In certain embodiments, the pathologically elevated level of protein in urine of the subject (e.g., a subject having SLE) may be reduced to a level that is closer to or similar to a control or reference, e.g., the level of a healthy subject or population. In certain embodiments, the level of protein in urine is maintained at a level similar to a control or reference, e.g., the level of a healthy subject or population.

In certain embodiments, the methods (e.g., for treating SLE) as described herein may further comprise detecting the expression level of prolactin and/or the expression level of LF/IF PRLR or total PRLR in a sample obtained from the subject prior to administration of the SMO. In certain embodiments, the sample is a PBMC sample. In certain embodiments, the sample comprises B cells or subset(s) thereof (e.g., plasma cells and/or their precursor B cells) as described herein.

Certain embodiments provide the use of a SMO as described herein to prepare a medicament for reducing the number of B cells (e.g., pathogenic, autoreactive B cells) in a subject in need thereof.

Certain embodiments provide a composition comprising a SMO as described herein for the prophylactic or therapeutic treatment of a B cell mediated disorder (e.g., SLE, or B cell malignancy).

Certain embodiments provide the use of a composition comprising a SMO as described herein to prepare a medicament for reducing the number of B cells (e.g., pathogenic, autoreactive B cells) in a subject in need thereof. Certain embodiments also provide a method of treating a cancer associated with the overexpression of AID, BCL2 and/or oncoprotein cMyc in a subject in need thereof, comprising administering a SMO as described herein to the subject, wherein the SMO inhibits the expression of LF/IF PRLR (e.g., inhibiting B cell expression of LF/IF PRLR) and/or reduces the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1).

In certain embodiments, such a method may be used to treat a cancer wherein the malignant transformation is associated with the overexpression of AID. In certain embodiments, the cancer is a B cell malignancy (e.g., a B cell malignancy described herein). In certain embodiments, the cancer is a gastric cancer.

In certain embodiments, such a method may be used to treat a cancer associated with the overexpression of BCL2.

In certain embodiments, such a method may be used to treat a cancer associated with the overexpression of oncoprotein c-Myc.

In certain embodiments, the method may further comprise administering one or more additional therapeutic agents as described herein.

Certain embodiments provide a SMO as described herein for treating a cancer associated with the overexpression of AID, BCL2 and/or oncoprotein cMyc in a subject in need thereof, wherein the SMO is capable of inhibiting the expression of LF/IF PRLR (e.g., inhibiting B cell expression of LF/IF PRLR) and/or is capable of reducing the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1).

Certain embodiments provide the use of a SMO as described herein for the preparation of a medicament for treating a cancer associated with the overexpression of AID, BCL2 and/or oncoprotein cMyc in a subject in need thereof, wherein the SMO is capable of inhibiting the expression of LF/IF PRLR (e.g., inhibiting B cell expression of LF/IF PRLR) and/or is capable of reducing the relative expression of LF/IF PRLR compared to the expression of one or more SF PRLRs (e.g., reduces relative B cell expression of LF/IF PRLR to one or more SF PRLRs). In certain embodiments, the SMO reduces the relative expression of LF/IF PRLR over total PRLR expression (e.g., LF+IF+SF, see Example 1).

In certain embodiments, such a SMO may be for use/used in combination with one or more additional therapeutic agents described herein.

### Splice Modulating Oligonucleotide (SMO)

Various forms of the prolactin receptor are produced by alternative splicing of the prolactin receptor pre-mRNA (Abramicheva, et al., Biochemistry (Mosc) 84(4):329-345 (2019); and Ormandy et al., DNA Cell Biol 17:761-70 (1998)). The long form of the human prolactin receptor (LF PRLR) protein is encoded by mRNA with exons 3-10 of PRLR pre-mRNA spliced together. The intermediate form of the human prolactin receptor (IF PRLR) is encoded by exons 3-9 plus 2 portions of exon 10 of PRLR pre-mRNA spliced together. The short form human PRLR splice variants comprise exons 3-9 and differing portions of exon 11 of the human prolactin receptor pre-mRNA spliced together, except that SF1a of the human PRLR also contains 154 nucleotides of exon 10 (accession numbers M31661 (LFPRLR), AF166329 (IF PRLR), AF416619 (SF1a), AF416618 (SF1b), GU133399 (SF1c), incorporated by reference herein). As described herein, LF/IF PRLR mRNA can be advantageously modulated (e.g., down-regulated) in a subject (e.g., in B cells) using a SMO (e.g., a SMO described herein).

As described herein, a SMO for use in the present invention is capable of inhibiting the expression of LF/IF PRLR; reducing the relative expression of LF/IF PRLR as compared to the expression of one or more SF PRLRs; and/or reducing the relative expression of LF/IF PRLR over total PRLR expression (e.g., LF+IF+SF). For example, the SMO may inhibit pre-mRNA splicing that produces the LF/IF PRLR. In certain embodiments, the SMO may hybridize to a target sequence in the PRLR pre-mRNA that comprises or overlaps at least one of: an intron-exon junction, a 5′splice site, a 3′splice site, a branch site, an intronic splice enhancer, an exonic splice enhancer, an intronic splice silencer or an exonic splice silencer. Methods for designing and synthesizing SMOs to modulate splicing are known in the art and described herein. For example, certain SMOs that may be used for LF/IF PRLR inhibition are described in U.S. Pat. No. 9,909,128, which is incorporated by reference herein for all purposes. Thus, in certain embodiments, a SMO for use herein is a SMO as described in U.S. Pat. No. 9,909,128.

In certain embodiments, the SMO spans an intron-exon junction to achieve exon-skipping during pre-mRNA processing, thus reducing the mRNA level of LF/IF PRLR (e.g., in favor of short forms of the receptor (SF PRLR)). In certain other embodiments, the SMO may hybridize to a binding site for a splicing protein, and thereby modulate splicing to reduce the mRNA level of LF/IF PRLR (e.g., in favor of short forms of the receptor (SF PRLR)).

In certain embodiments, the SMO is capable of hybridizing to a target sequence that spans the intron 9/exon10 junction of the PRLR pre-mRNA and reducing the mRNA level of LF/IF PRLR. In certain embodiments, the SMO is capable of hybridizing to target sequence UGUGUCUGUGUUUUAAUAGAAGGGC (SEQ ID NO:6) from human PRLR pre-mRNA, or to a corresponding sequence from a different species (e.g., from mouse).

In certain embodiments, the SMO is capable of hybridizing to a target sequence that is present in intron 9 of the PRLR pre-mRNA and reducing the mRNA level of LF/IF PRLR. In certain embodiments, the SMO is capable of hybridizing to a target sequence that is present in exon 10 of the PRLR pre-mRNA and reducing the mRNA level of LF/IF PRLR. In certain embodiments, the target sequence present in intron 9 or exon 10 is a binding site, or a portion thereof, for a splicing protein. In certain embodiments, the SMO is capable of hybridizing to a target sequence from human PRLR pre-mRNA selected from the group consisting of: CUUGAGUU, CCCUCUAG, and GUACUGUUUUU (SEQ ID NO:9), or to a corresponding sequence from a different species (e.g., from mouse).

As described herein, SF1a mRNA includes a small 154 nucleotide portion of exon 10, but not the intact full-length exon 10. Therefore, it is theoretically possible that, in addition to modulating LF/IF PRLR mRNA, SF1a mRNA may also be modulated by certain SMOs in some tissues (e.g., certain SMOs that modulate intron 9-exon 10 splicing activity). In particular, such modulation would be dependent on, e.g., splicing factor regulation in a given tissue and expression of SF1a in said tissue. For example, regulation of splicing by the human form of Antimaia does not appear to downregulate SF1a mRNA expression in SU-DHL-6 and Karpas-422 cells, potentially due to splicing factor regulation.

Thus, in certain embodiments, a SMO as described herein is also capable of reducing the mRNA level of SF1a (e.g., in certain tissues). In certain embodiments, the SMO is more efficient in reducing LF/IF PRLR mRNA as compared to reducing SF1a PRLR mRNA (if at all) in a B cell described herein, thereby, reducing the relative expression of LF/IF PRLR mRNA compared to SF1a PRLR mRNA. In other embodiments, a SMO as described herein does not reduce the mRNA level of SF1a (e.g., in certain tissues). In certain embodiments, a SMO as described herein does not reduce SF1a PRLR mRNA in a B cell described herein. In certain embodiments, a SMO as described herein inhibits splicing that is needed for the inclusion of more than 154 nucleotides of exon 10 in a mature mRNA.

In certain embodiments, the oligonucleotide is a modified oligonucleotide (e.g., comprises synthetic, non-natural or altered bases, sugars and/or backbone modifications). In certain embodiments, the oligonucleotide comprises one or more modified nucleosides or nucleotides. In certain embodiments, the oligonucleotide comprises a modified backbone. For example, in certain embodiments, the oligonucleotide is a morpholino oligonucleotide (i.e., a phosphorodiamidate morpholino oligomer (PMO)). As used herein, a morpholino oligomer has a backbone of methylenemorpholine rings linked through phosphorodiamidate groups.

Antimaia is the name given to one embodiment of a splice-modulating oligomer (used in Example 1, which is referenced therein as LFPRLR SMO, wherein the mouse form of Antimaia corresponds to mLFPRLR SMO and the human form of Antimaia corresponds to hLFPRLR SMO (see, Example 1, Table s1a) that is capable of inhibiting the expression of the LF/IF prolactin receptor, and by doing so, may increase the relative expression of the short form prolactin receptors. Accordingly, the proportion of LF/IF PRLR as compared to total PRLR may be reduced and the proportion of SF PRLR(s) as compared to total PRLR may be increased. Human and mouse forms of Antimaia have the following sequences: 5′-GCCCTTCTATTAAAACACAGACACA-3′(human, SEQ ID NO:1); 5′-GCCCTTCTATTGAAACACAGATACA-3′ (mouse, SEQ ID NO:2). Antimaia, is a modified oligonucleotide and comprises morpholino groups to enhance stability/half-life. As such, the bases shown in SEQ ID NOS:1 and 2 are attached to a backbone of methylenemorpholine rings that are linked through phosphorodiamidate groups. Additionally, Antimaia is a Vivo-Morpholino oligonucleotide, wherein an octaguanidine dendrimer is attached via a linker group to the last methylenemorpholino ring associated with the 3′ terminal residue of the oligonucleotide and the terminal phosphorodiamidate group at the 5′ end of the oligonucleotide has a tertiary amine that is capped with —CH₂C(═O)NH₂. In certain embodiments, a vivo-morpholino oligonucleotide has the structure of formula I:

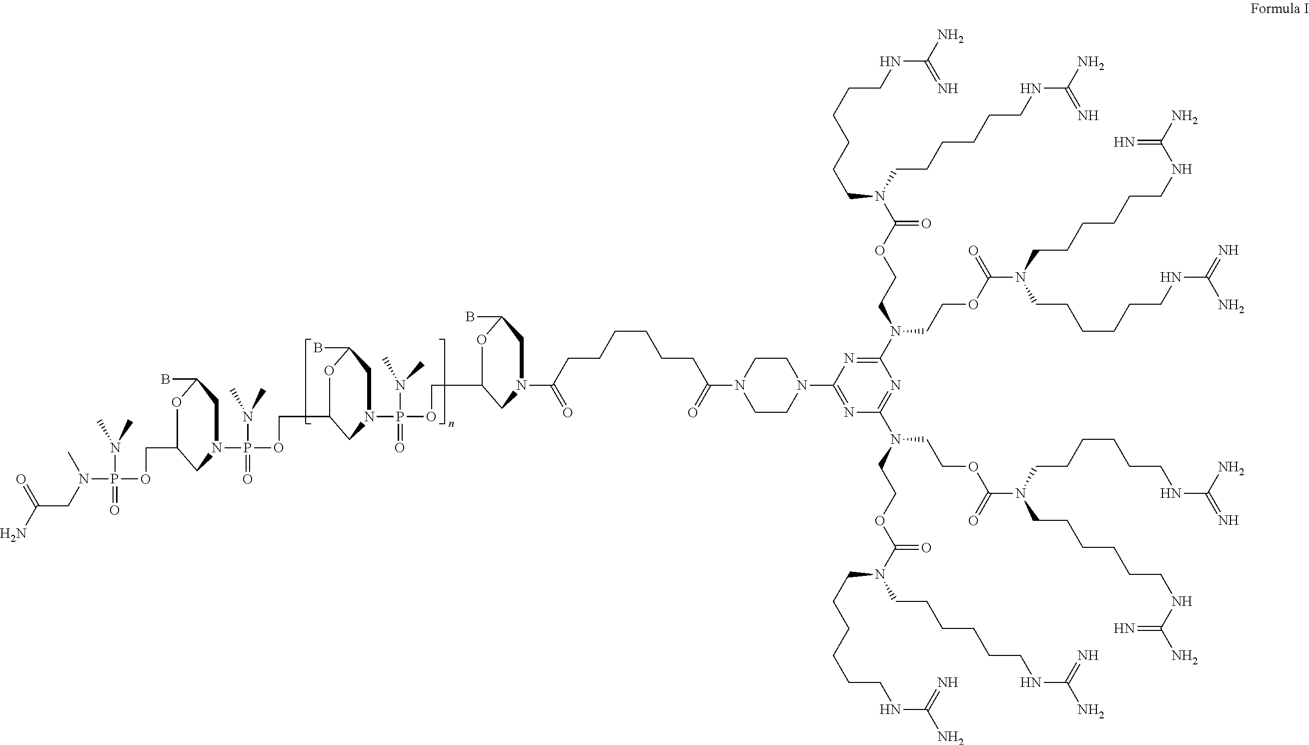

wherein each B is independently a base according to a given oligonucleotide sequence (e.g., SEQ ID NO:1 or 2), and n is an integer of from 5 to 98. The length of the oligonucleotide in formula I is n+2. For example, n is 23 when the length of the oligonucleotide is 25 nt (e.g., SEQ ID NO:1 or 2). In certain embodiments, n is from about 5 to 80, 6 to 70, 7 to 60, 8 to 50, or 9 to 40. In certain embodiments, n is from about 5 to 35, 10 to 30, or 15 to 25.

Thus, in certain embodiments, the oligonucleotide (e.g., the morpholino oligonucleotide) comprises a nucleic acid sequence having at least about 85% sequence identity to GCCCTTCTATTAAAACACAGACACA (SEQ ID NO:1). In certain embodiments, the oligonucleotide (e.g., the morpholino oligonucleotide) comprises a nucleic acid sequence having at least about 86%, 87%, 88%, 89%, 90%, 91%, 92%, 93%, 94%, 95%, 96%, 97%, 98%, 99%, or 100% identity to SEQ ID NO:1. In certain embodiments, the oligonucleotide (e.g., the morpholino oligonucleotide) comprises a nucleic acid sequence having at least about 90% sequence identity to SEQ ID NO:1. In certain embodiments, the oligonucleotide (e.g., the morpholino oligonucleotide) comprises a nucleic acid sequence having at least about 95% sequence identity to SEQ ID NO:1. In certain embodiments, the oligonucleotide (e.g., the morpholino oligonucleotide) comprises SEQ ID NO:1. In certain embodiments, the oligonucleotide (e.g., the morpholino oligonucleotide) consists of SEQ ID NO:1.

In certain embodiments, the oligonucleotide (e.g., the morpholino oligonucleotide) comprises a nucleic acid sequence having at least about 85% sequence identity to GCCCTTCTATTGAAACACAGATACA (SEQ ID NO:2). In certain embodiments, the oligonucleotide (e.g., the morpholino oligonucleotide) comprises a nucleic acid sequence having at least about 86%, 87%, 88%, 89%, 90%, 91%, 92%, 93%, 94%, 95%, 96%, 97%, 98%, 99%, or 100% identity to SEQ ID NO:2. In certain embodiments, the oligonucleotide (e.g., the morpholino oligonucleotide) comprises a nucleic acid sequence having at least about 90% sequence identity to SEQ ID NO:2. In certain embodiments, the oligonucleotide (e.g., the morpholino oligonucleotide) comprises a nucleic acid sequence having at least about 95% sequence identity to SEQ ID NO:2. In certain embodiments, the oligonucleotide (e.g., the morpholino oligonucleotide) comprises SEQ ID NO:2. In certain embodiments, the oligonucleotide (e.g., the morpholino oligonucleotide) consists of SEQ ID NO:2.

In certain embodiments, the oligonucleotide comprises a nucleic acid sequence having at least about 85% sequence identity to AACTCAAG. In certain embodiments, the oligonucleotide comprises a nucleic acid sequence having at least about 85% sequence identity to CTAGAGGG.

In certain embodiments, the oligonucleotide comprises a nucleic acid sequence having at least about 85% sequence identity to AAAAACAGTAC (SEQ ID NO:5).

In certain embodiments, the oligonucleotide (e.g., the morpholino oligonucleotide) comprises a nucleic acid sequence having at least about 86%, 87%, 88%, 89%, 90%, 91%, 92%, 93%, 94%, 95%, 96%, 97%, 98%, 99%, or 100% identity sequence identity to SEQ ID NO: 3, 4, or 5.

In certain embodiments, an oligonucleotide described herein is operably linked (e.g., directly or via a linking group) to a cell penetrating moiety. For example, in certain embodiments, the oligonucleotide is operably linked to an octaguanidine dendrimer. In certain embodiments, the cell penetrating moiety is linked (e.g., via a linking group) to the 5′ end of the oligonucleotide. In certain embodiments, the cell penetrating moiety is linked (e.g., via a linking group) to the 3′ end of the oligonucleotide.

In certain embodiments, the cell penetrating moiety comprises a dendrimer. For example, the cell penetrating moiety comprises an octaguanidine dendrimer. Exemplary intracellular drug delivery technology and octaguanidine dendrimers are also described in U.S. Pat. No. 8,637,492, Moulton et al., Molecules, 14, 1304-1323(2009). doi:10.3390/molecules14031304, and Morcos, et al., Biotechniques. 45(6):613 (2008). doi: 10.2144/000113005, which are incorporated by reference herein.

In certain embodiments, the cell penetrating moiety comprises a cell penetrating peptide (e.g., a cationic CPP, such as TAT peptide having amino acid sequence of GRKKRRQRRRPQ (SEQ ID NO:10)).

In certain embodiments, the oligonucleotide is operably linked (e.g., directly or via a linking group) to a cell penetrating peptide.

In certain embodiments, the oligonucleotide is comprised within an intracellular delivery system. In certain embodiments, the oligonucleotide is comprised within a nanoparticle. In certain embodiments, the oligonucleotide is comprised within a lipid nanoparticle.

In certain embodiments, a SMO as described herein is incorporated into a composition that further comprises a carrier. In certain embodiments, the composition is a pharmaceutical composition comprising a pharmaceutically acceptable carrier. In certain embodiments, the composition is a liquid composition. In certain embodiments, the composition is a solid composition (e.g., powder or lyophilized formulation). In certain embodiments, the composition is a lyophilized composition that further comprises one or more excipients selected from the group consisting of a cryo-lyoprotectant (e.g., trehalose, sucrose) and a bulking agent (e.g., mannitol, glycine). In certain embodiments, the solid composition may be reconstituted (e.g., with water, saline or Dextrose solution) prior to use.

Certain embodiments also provide a kit comprising a SMO as described herein, packaging material, and instructions for administering the SMO to a mammal in need thereof to prevent or treat a B cell mediated disorder (e.g., instructions for administering the SMO to a mammal selected for treatment or identified as a candidate for treatment). In certain embodiments, the kit comprises a syringe (e.g., a pre-filled syringe) or a vial comprising the composition as described herein. In certain embodiments, the kit further comprises a needle that is or could be fitted with the syringe (e.g., to deliver subcutaneous, intradermal or intramuscular injection).

### Additional Therapies and Agents

In certain embodiments, a method described herein further comprises administering one or more additional therapeutic agents or therapies to the subject.

In certain embodiments, the additional therapeutic agents or therapies are useful for treating a B cell mediated disorder. In certain embodiments, the additional therapeutic agents or therapies are useful for treating a B cell malignancy. In certain embodiments, the additional therapeutic agents or therapies are useful for treating an autoimmune disease, such as SLE, RA, or SS.

In certain embodiments, the one or more additional therapeutic agents or therapies are selected from the group consisting of cyclophosphamide, doxorubicin, vincristine, prednisone, anti-B cell antibody, anti-B cell CAR-T, NK cell-based therapy, checkpoint inhibitor, stem cell transplantation, a BCL2 inhibitor (e.g., Venetoclax), and combinations thereof. In certain embodiments, the additional therapeutic agent is a chemotherapeutic agent, such as doxorubicin, or vincristine. In certain embodiments, the additional therapeutic agent is a corticosteroid, such as prednisone. In certain embodiments, the additional therapeutic agent is an antibody or antibody drug conjugate (ADC), such as anti-CD20 or anti-CD19 antibody, or an ADC thereof. In certain embodiments, the additional therapy is a cancer immunotherapy. In certain embodiments, the additional therapy is an antibody-based cancer immunotherapy. In certain embodiments, the additional therapy is an immune cell-based cancer immunotherapy (e.g., T or NK cell therapy). In certain embodiments, the additional therapy is a NK cell-based therapy, such as an NK cell-based therapy as described in Kumar et al. Generation and validation of CRISPR-engineered human natural killer cell lines for research and therapeutic applications. STAR Protocols, Volume 2, Issue 4, 17 Dec. 2021, 100874, which is incorporated by reference herein. In certain embodiments, the additional therapy is a checkpoint inhibitor, such as an anti-CTLA4, anti-PD1 or anti-PDL1 agent. In certain embodiments, the additional therapeutic agent is a Chimeric Antigen Receptor (CAR)-T cell, such as a BCMA-directed CAR T. In certain embodiments, the additional therapy is stem cell transplantation. In certain embodiments, the additional therapy is a Myc inhibitor. In certain embodiments, the one or more additional therapeutic agents or therapies are selected from the group consisting of cyclophosphamide, doxorubicin, vincristine, prednisone, an anti-B cell antibody, an anti-B cell CAR-T, a NK cell-based therapy, a checkpoint inhibitor, stem cell transplantation, a nonsteroidal anti-inflammatory drug (NSAID), a corticosteroid, an immunosuppressant, an antimalarial drug, an anti-B cell activating factor (BAFF) antibody, a Myc inhibitor, a BCL2 inhibitor, an anti-psychotic and combinations thereof. In certain embodiments, the one or more additional therapeutic agents or therapies are selected from the group consisting of cyclophosphamide, doxorubicin, vincristine, prednisone, an anti-B cell antibody, an anti-B cell CAR-T, a NK cell-based therapy, a checkpoint inhibitor, stem cell transplantation, a nonsteroidal anti-inflammatory drug (NSAID), a corticosteroid, an immunosuppressant, an antimalarial drug, an anti-B cell activating factor (BAFF) antibody, a Myc inhibitor, a BCL2 inhibitor, an anti-psychotic, a proteasome inhibitor, an anti-plasma cell agent (e.g., anti-CD138) and combinations thereof. In certain embodiments, the one or more additional therapeutic agents include a proteasome inhibitor (e.g., bortezomib) or other anti-plasma cell agents, which may be used to treat multiple myeloma, plasmacytoma or other plasma cell malignancies (e.g., anti-CD138).

In certain embodiments, the one or more additional therapeutic agents or therapies are selected from the group consisting of a nonsteroidal anti-inflammatory drug (NSAID), a corticosteroid, an immunosuppressant, a vinca alkaloid, L-asparaginase, a DNA intercalating anthracycline, a DNA alkylating nitrogen mustard, methotrexate, an antimalarial drug, an anti-B cell activating factor (BAFF) antibody, an anti-B cell antibody, a BCL2 inhibitor (e.g., Venetoclax), and combinations thereof.

As dopamine antagonists elevate prolactin, they may be contributing to the increasing incidence of SLE and/or DLBCL. Therefore, dopamine antagonists and drugs that inhibit prolactin secretion may be contraindicated in subjects having certain B cell mediated disorders, such as SLE or DLBCL. Unlike drugs that inhibit prolactin, the long/intermediate form receptor specific intervention methods described herein (using a SMO such as Antimaia) could be combined with anti-psychotics (e.g., dopamine antagonists) that elevate prolactin. Thus, one could effectively treat lupus and/or DLBCL in patients with psychiatric and post-traumatic stress disorders. Accordingly, in certain embodiments, a method described herein further comprises administering an anti-psychotic (e.g., dopamine antagonists).

In certain embodiments, the one or more additional therapeutic agents is a SMO as described herein, which is different from the initial SMO administered.

The one or more additional therapeutic agents may be administered either simultaneously or sequentially with the SMO. In certain embodiments, the one or more additional therapeutic agents is administered simultaneously with the SMO. In certain embodiments, a pharmaceutical composition comprising the SMO and the at least one other therapeutic agent is administered. In certain embodiments, SMO and the one or more additional therapeutic agents are administered sequentially. In certain embodiments, the SMO is administered first and one or more additional therapeutic agents is administered second. In certain embodiments, the one or more additional therapeutic agents is administered first and the SMO is administered second.

### Diagnostic and Screening Methods

As described herein, LF/IF PRLR and/or prolactin expression levels may be used as biomarkers of B cell mediated disease (e.g., SLE, B cell malignancy), and therefore, may be used, e.g., to identify or select subjects that are a candidate for treatment and/or that are likely to benefit from treatment; to monitor disease progression and/or to identify subjects at a higher risk of developing a B cell malignancy; to monitor response to therapy, and optionally, be used to adjust a subject's treatment regimen; and to screen test agents for activity as B cell mediated disease therapies. In particular, elevated levels of LF/IF PRLR and/or B cell-derived prolactin as compared to a control may be indicative of a B cell mediated disorder (e.g., SLE, B cell malignancy), or a higher likelihood of developing a B cell malignancy, such as DLBCL; may be indicative of a subject that is a candidate for treatment or that is likely to benefit from treatment; and/or may be indicative that a subject is not responding to a treatment or is in need of a treatment modification, such as an increase in dose.

Accordingly, steps of evaluating LF/IF PRLR and/or prolactin expression levels in a sample from a patient may be incorporated into a method described herein. For example, expression levels may be evaluated prior to administration of a therapy (e.g., a SMO described herein, such as Antimaia), after administration of a therapy or at both time points.

Expression levels may also be evaluated as an initial step and a therapy may or may not be administered in conjunction with the evaluation. For example, certain embodiments provide a method of identifying and/or selecting a subject as a candidate for treatment with a therapy for treating a B cell mediated disorder (e.g., SLE, and/or a B cell malignancy), comprising detecting the expression level of prolactin and/or LF/IF PRLR in a sample from the subject, wherein the subject is identified and/or selected as a candidate for treatment when the expression level of prolactin (e.g., blood or serum level of prolactin, or B cell-derived prolactin, or PRL of non-B cell origin within the immune microenvironment) and/or LF/IF PRLR is higher than a control value, and wherein the identified subject is optionally administered a therapy to treat a B mediated disorder, such as a SMO described herein.

Certain embodiments also provide a method of identifying a subject as having a higher likelihood of developing a B cell mediated disorder (e.g., a B cell malignancy such as DLBCL, B-CLL, or B-ALL, or follicular lymphoma), the method comprising detecting the expression level of prolactin (e.g., blood or serum level of prolactin, or B cell-derived prolactin, or PRL of non-B cell origin within the immune microenvironment) and/or LF/IF PRLR in a sample from the subject, wherein the subject is identified as having a higher likelihood of developing a B cell mediated disorder (e.g., a B cell malignancy) when the expression level of prolactin and/or LF/IF PRLR is higher than a control value, and wherein the identified subject is optionally administered a therapy to treat a B cell mediated disorder (e.g., a B cell malignancy), such as a SMO described herein. For example, as described herein, subjects with certain autoimmune disorders, such as SLE, are at an increased risk of developing a B cell malignancy. Thus, in certain embodiments, the subject having a higher likelihood of developing a B cell mediated disorder has an autoimmune disorder, such as SLE (e.g., having hyperprolactinemia). In certain embodiments, the subject is an infant that has pre-leukemic B cell clone(s) (e.g., identified from newborn blood spot test. Such subject might be predisposed to developing B-ALL). The subject as described herein could be selected for monitoring and/or for early intervention, for example, by administering a LF/IF PRLR inhibition SMO as described herein, optionally in combination with one or more additional therapeutic agents.

In certain embodiments, LF/IF PRLR levels are evaluated. For example, in certain embodiments, a method described herein may comprise the steps of detecting the expression level of LF/IF PRLR in a sample (e.g., a PBMC sample, or B cells sample as described herein such as plasma cells and/or their precursor B cells) obtained from the subject and identifying the subject as likely to benefit from administration of a therapy to treat a B cell mediated disorder (e.g., a SMO described herein, such as Antimaia) when increased expression levels are detected. For example, if the subject has an increased expression level of LF/IF PRLR (mRNA and/or protein) compared to a control (e.g., a control reference level from a sample of a healthy person, or from a sample of a patient cohort with a B cell disorder with less aggressiveness and/or better prognosis), the subject may be identified as having an increased risk of exacerbation of a B cell disorder (e.g., lupus and/or DLBCL) and thus likely to benefit from administration of a therapy described herein. In certain embodiments, a lupus patient could be identified as having an increased risk of developing a B cell lymphoma (e.g., DLBCL) if the patient has an increased expression of LF/IF PRLR compared to a control. In certain embodiments, a lupus patient could be identified as likely to benefit from administration of a therapy to treat a B cell mediated disorder such as SLE (e.g., using a SMO described herein, such as Antimaia) if the patient has an increased expression of LF/IF PRLR or total PRLR in a sample (e.g., a PBMC sample, or B cell sample such as plasma cells described herein and/or precursor B cells capable of giving rise to plasma cells described herein) compared to a control.

In certain embodiments, prolactin levels are evaluated (e.g., by a method known in the art or described herein, such as by an ELISA assay or PCR). For example, in certain embodiments, a method described herein may comprise the steps of detecting the level of expression of prolactin in a sample obtained from a subject and identifying the subject as likely to benefit from administration of a therapy to treat a B cell mediated disorder (e.g., a SMO described herein, such as Antimaia) when increased expression levels are detected. For example, if the subject has an increased level of prolactin expression (mRNA and/or protein level) compared to a control (e.g., a control reference level of a sample from a healthy person, or from a sample of a patient cohort of a B cell disorder with less aggressiveness and/or better prognosis), the subject may be identified as having an increased risk of aggravation of a B cell disorder (e.g., lupus and/or DLBCL) and thus likely to benefit from administration of the therapy described herein.

In certain embodiments, a method described herein further comprises optionally detecting one or more downstream factors impacted by the prolactin-LF/IF PRLR signaling (e.g., pSTAT3, Myc, Bcl2, TCL1, and/or AID) in a sample obtained from the subject prior to administering a B cell mediated therapy. If the subject has an increased expression level (mRNA and/or protein) compared to a control (e.g., a control reference level of a sample from a healthy person, or from a sample of a patient cohort of a B cell disorder with less aggressiveness and/or better prognosis), the subject may be identified as having an increased risk of aggravation of a B cell disorder (e.g., lupus and/or DLBCL) and thus would likely to benefit from administration of a therapy described herein.

In certain embodiments, a treatment method or a diagnostic/screening method described herein comprises detecting constitutive overexpression of both Myc and Bcl2 (e.g., chromosomal translocation of both Myc and Bcl2, such as IGH-Myc and IGH-Bcl2 chromosomal translocation) in a B cell (e.g., a malignant B cell such as B-ALL or DLBCL). If the detected B cell in the subject does not have constitutive overexpression of both Myc and Bcl2 (e.g., no chromosomal translocation of both Myc and Bcl2), the subject may be identified as likely to benefit from administration of a LF/IF PRLR inhibition SMO as described herein. Alternatively, if the detected B cell in the subject has constitutive overexpression of both Myc and Bcl2, the subject may be identified as potentially less sensitive to a LF/IF PRLR inhibition SMO as described herein as compared to a subject lacking constitutive overexpression of both Myc and Bcl2, and may benefit from increased dosing and/or a combination therapy. Thus, in certain embodiments, if the detected B cell in the subject has constitutive overexpression of both Myc and Bcl2, the subject may be identified as a candidate for an increased dose of a LF/IF PRLR inhibition SMO as described herein and/or as a candidate for a combination therapy, wherein a LF/IF PRLR inhibition SMO as described herein is administered in combination with an additional therapy described herein.

In certain embodiments, the sample is a blood sample (e.g., an intravenous blood sample, or prick blood sample such as blood spot sample of a newborn). In certain embodiments, the sample comprises peripheral blood mononuclear cells (PBMCs). In certain embodiments, the sample comprises B cells (e.g., B cell sample such as plasma cells described herein and/or precursor B cells capable of giving rise to plasma cells described herein). In certain embodiments, the sample comprises abnormal B cells. In certain embodiments, the sample is a biopsy sample (e.g., any B cell lymphoma or lupus lesion biopsy from a tissue/organ such as lymph nodes, skin, bone marrow, or spleen).

In certain embodiments, B cells or subset thereof, or B cell derived marker (e.g., prolactin, PRLR, AID, Myc, TCL1, or Bcl2) may be characterized using any suitable assay methods (e.g., flow cytometry, RT-PCR, western blot, ELISA, or sequencing), optionally after isolation from a biological sample. For example, B cells may be probed after optional enrichment or separation step(s) using suitable technique such as fluorescence activated cell sorting (FACS) and/or the magnetic activated cell sorting (MACS).

Certain embodiments also provide a method for screening a test agent for activity as a treatment for a B cell mediated disorder (e.g., DLBCL), the method comprising contacting an abnormal B cell (e.g., a DLBCL cell) with the test agent and measuring the expression of LF/IF PRLR, wherein a decrease in the expression of LF/IF PRLR as compared to the expression of LF/IF PRLR in a control cell indicates the test agent has activity as a treatment for a B cell mediated disorder (e.g., DLBCL).

### Administration

Compounds described herein (e.g., SMOs or other therapeutic agents) can be formulated as pharmaceutical compositions and administered to a mammalian host, such as a human patient in a variety of forms adapted to the chosen route of administration, i.e., orally or parenterally, by intravenous, intradermal, intramuscular, intraperitoneal, intrathecal, topical, nasal, inhalation, pulmonary, suppository, sub dermal osmotic pump, or subcutaneous routes. In certain embodiments, a compound described herein is administered intratumorally, such as via one or more intratumoral or intranodal injections. In certain embodiments, a compound described herein is administered via an implant, such as an implanted pump.

Thus, compounds may be systemically administered, e.g., orally, in combination with a pharmaceutically acceptable vehicle such as an inert diluent or an assimilable edible carrier. They may be provided as a lyophilized formulation (e.g., with trehalose or sucrose as cryo-lyoprotectant, and/or mannitol as bulking agent), or enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered subcutaneously, intradermally, intranasally, intramuscularly, intrathecally, intravenously or intraperitoneally by infusion or injection. A compound may also be administered via intranasal and/or pulmonary delivery (e.g., delivered as a spray or mist). Additionally, the compound may be administered by local injection, such as by intrathecal injection, epidural injection or peri-neural injection using a scope. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds described herein to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of the invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound required for use in treatment will vary with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Implantable drug delivery devices, also referred to as implanted pumps or drug delivery pumps, may be used to administer therapeutic agents to various locations within the body. Some drug delivery devices have variable volumetric flow rates, adjustable through an external programmer device. Other implantable drug delivery devices have fixed volumetric flow rates, but can be activated and deactivated externally. Still other drug delivery devices have fixed volumetric flow rates and are not adapted to be controlled from outside of the body. For example, implantable, electromechanical drug delivery devices can include, within a fluid impermeable and sealed casing, a watch-type drive mechanism that drives a circular wheel. Microchip based drug delivery devices are also available, and can include a plurality of drug reservoirs etched into a substrate to produce, for example, a single microchip.

The SMOs described herein can also be administered in combination with other therapeutic agents or therapies. For example, the SMOs may be administered with other agents that are useful for treating a B cell mediated disorder (e.g., a B cell malignancy or SLE). Accordingly, in one embodiment the invention also provides a composition comprising a SMO as described herein, at least one other therapeutic agent (e.g., an immune cell therapy such as a CAR-T or NK therapy), and a pharmaceutically acceptable diluent or carrier. The invention also provides a kit comprising a SMO described herein, one or more additional therapeutic agents and instructions for administering the SMO and the other therapeutic agent or agents to a subject for the treatment of a B cell mediated disorder.

### Certain Definitions

As used herein, the term, “splice modulating oligonucleotide” or SMO refers to an oligonucleotide, 7-100 nucleotides in length, characterized by having a nucleotide sequence that is complementary to a target nucleotide sequence in a pre-mRNA present in a mammalian cell and renders the oligonucleotide operative, when contacted with the mammalian cell in therapeutically effective amounts, to modulate (e.g., up-regulate or down-regulate) an amount of a mRNA splice variant produced from the pre-mRNA in the cell or to increase a degradation rate of the pre-mRNA in the cell and/or decrease a translation rate of a mRNA. In embodiments in which SMOs modulate splicing of a pre-mRNA, such SMOs may comprise a nucleotide sequence complementary to a target sequence in the pre-mRNA that comprises or overlaps: an intron-exon junction; an intronic or exonic splice silencer; and/or an intronic or exonic splice enhancer. In embodiments in which SMOs increase a pre-mRNA degradation rate or decrease a mRNA translation rate, such SMOs may comprise a nucleotide sequence complementary to one or more target sequence(s) in the pre-mRNA that comprise(s) or overlap(s) a nucleotide sequence from a single exon or two or more nucleotide sequences from two or more exons.

The nucleotides of SMOs can be modified or unmodified DNA nucleotides, RNA nucleotides, or combinations thereof. In some embodiments, a SMO composition comprises at least one of a DNA oligonucleotide, an RNA oligonucleotide, a morpholino oligonucleotide, or a “vivo morpholino” oligonucleotide. In particular, the oligonucleotide can include modified oligonucleotide backbones such as, but not limited to, phosphorothioates, phosphorodiamidates, chiral phosphorothioates, phosphorodithioates, phosphotriesters, aminoalkylphosphotriesters, methyl and other alkyl phosphonates 3′-alkylene phosphonates and chiral phosphonates, phosphinates, phosphoramidates 3′-amino phosphoramidate and aminoalkylphosphoramidates, thionophosphoramidates, thionoalkylphosphonates, thionoalkyl phosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to various salts, mixed salts and free acid forms are also included. References that teach the preparation of such modified backbone oligonucleotides are provided, for example, in U.S. Pat. Nos. 4,469,863 and 5,750,666, all incorporated by reference herein. The design and synthesis of antisense oligonucleotides is well known in the art. Computer programs for the design of antisense oligonucleotide sequences are also available.

In some embodiments, at least seven, eight, 10, 15, 20, 25, 30, 40, or 50 contiguous nucleotides in a SMO are complementary to the target nucleotide sequence in the selected pre-mRNA that comprises, or overlaps, a sequence motif selected from the group consisting of a 5′ splice site, a 3′ splice site, a branch site, an intronic splice enhancer, an intronic splice silencer, an exonic splice enhancer, and an exonic splice silencer. In some embodiments, at 8-12, 8-15, 8-20, 10-25, 15-30, 20-40, or 30-50 contiguous nucleotides in a SMO are complementary to the target nucleotide sequence in the selected pre-mRNA that comprises, or overlaps, a sequence motif selected from the group consisting of a 5′ splice site, a 3′ splice site, a branch site, an intronic splice enhancer, an intronic splice silencer, an exonic splice enhancer, and an exonic splice silencer.

In some embodiments, a SMO is 7-50 nucleotides in length, 7-25 nucleotides in length, 7-15 nucleotides in length, 8-12 nucleotides in length, 8-17 nucleotides in length, 8-25 nucleotides in length, 8-50 nucleotides in length, 9-12 nucleotides in length, 9-25 nucleotides in length, 10-17 nucleotides in length, 10-25 nucleotides in length, 20-30 nucleotides in length, or 25 nucleotides in length. In embodiments where a SMO comprises at least 75% RNA nucleotides, the SMO can be less than 21 nucleotides in length. In some embodiments, a SMO is entirely single-stranded. In some embodiments, a SMO is partially single-stranded. In some embodiments, a SMO is capable of reversibly or permanently forming stem loop structures, hairpin structures, and the like.

As used herein, the term “intron-exon junction” refers to sequences of nucleotides in a pre-mRNA that reside at the junction of an intron and an exon in the pre-mRNA. An intron-exon junction may extend up to 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, and 50 nucleotides in both 5′-ward and 3′-ward directions from the intron-exon border.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Modified nucleic acids (e.g., morpholino oligonucleotides) as described herein may have enhanced stability or resistance against enzymatic degradation. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention.

The terms “chemical modification”, “modification” or “modified” may refer to a chemical change in a compound when compared to its naturally occurring counterpart. For example, a nucleobase, a sugar moiety or an internucleoside linkage may be chemically modified.

The term “modified oligonucleotide” may refer to such sequences that contain synthetic, non-natural or altered bases, sugars and/or backbone modifications.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

“Expression” refers to the transcription and/or translation in a cell of an endogenous gene, transgene, as well as the transcription and stable accumulation of sense (mRNA) or functional RNA. Expression may also refer to the production of protein.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, J M B, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

As used herein, the phrase “sample” or “physiological sample” is meant to refer to a biological sample obtained from a subject that contains a protein or nucleic acid of interest (e.g., prolactin or LF/IF PRLR protein or RNA). Thus, the sample may be evaluated at, e.g., the nucleic acid or protein level. In certain embodiments, the physiological sample comprises, e.g., tissue, blood, serum, or plasma. In certain embodiments, the sample may include a fluid. In certain embodiments, the sample comprises blood (e.g., whole blood), serum and/or plasma. In certain embodiments, the sample comprises serum. In certain embodiments, the sample comprises blood. In certain embodiments, the sample comprises peripheral blood mononuclear cells (PBMCs). In certain embodiments, the sample comprises B cells. In certain embodiments, the sample comprises abnormal B cells. In certain embodiments, the sample comprises a tissue sample (e.g., obtained from, e.g., lymph nodes, spleen, bone marrow, liver, skin, joint, lymphoid tissues, such as mucosa associated lymphoid tissue and skin associated lymphoid tissue, and other organs/tissues into which malignant B cells may infiltrate or metastasize). In certain embodiments, the tissue sample is from a B cell lymphoma or lupus lesion biopsy from a tissue/organ, such as lymph nodes or skin).

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient). In certain embodiments, the patient has a B cell malignancy (e.g., a B cell NHL, CLL, or B-ALL) and/or autoimmune diseases (e.g., SLE, RA, or SS).

As described herein, levels of a protein or nucleic acid of interest may be compared to control or reference levels. Such a control or reference level may vary. For example, in certain embodiments, the term “control” may refer to a heathy subject or a subject that does not have a B cell mediated disorder or have a higher likelihood of developing such a disorder. Alternatively, the term “control” may refer to a subject that has a B cell mediated disorder that was not administered a treatment described herein (e.g., a SMO described herein) or to the subject prior to treatment (or a sample therefrom). Similarly, a “reference level” may refer to a level or a range of levels that is measured in, e.g., a healthy individual or in a subject that does not have a B cell mediated disorder or a higher likelihood of developing such a disorder. In certain other embodiments described herein, a “reference level” may also refer to a level or a range of levels in a subject that has a B cell mediated disorder that was not administered a treatment described herein (e.g., a SMO described herein) or to the subject prior to treatment (or a sample therefrom).

In certain embodiments, a control or reference level may be established using data from a population of control subjects. In some embodiments, the population of subjects is matched to a test subject according to one or more patient characteristics such as age, sex, ethnicity, or other criteria. In some embodiments, the control level is established using the same type of sample from the population of subjects as is used for assessing the levels in the test subject.

The term “subject,” “individual,” and “patient,” as used interchangeably herein, refer to a mammal, including but not limited to humans, higher non-human primates, rodents, domestic, cows, horses, pigs, sheep, dogs and cats. In one embodiment, the subject is a human.

The term “subject in need thereof” includes subjects that have been diagnosed with a B cell mediated disorder or are suspected of having such a disorder, as well as subjects that have been identified as having an above average likelihood of developing such a disorder. For example, in certain embodiments, the subject has been diagnosed with, or is suspected of having, a disease or condition that is associated with an abnormal B cell, or is identified as being at a higher risk of developing such a condition. In certain embodiments, the subject has been diagnosed with, or is suspected of having, an autoimmune disease or condition (e.g., SLE). In certain embodiments, the “subject in need thereof” is a subject that has been diagnosed as having an above average likelihood of developing a B cell malignancy, such as B cell lymphoma (e.g., non-Hodgkin's lymphoma such as DLBCL). In certain embodiments, the “subject in need thereof” is a subject that has been diagnosed as having B cell malignancy, such as a B cell lymphoma (e.g., DLBCL). In certain embodiments, the “subject in need thereof” is a subject that has been diagnosed as having SLE or suspected to have SLE. In certain embodiments, the subject has a B cell malignancy, such as B cell lymphoma. In certain embodiments, the subject has non-Hodgkin's lymphoma. In certain embodiments, the subject has DLBCL. In certain embodiments, the subject has SLE. In certain embodiments, the subject is suspected to have SLE. In certain embodiments, the subject has B cell lymphoma (e.g., DLBCL), which progressed from an autoimmune disorder (e.g., SLE).

The term “therapeutically effective amount,” in reference to treating a disease state/condition, refers to an amount of a therapeutic agent alone (e.g., a SMO described herein) or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as a B cell mediated disorder (e.g., SLE, aggravation of SLE, and/or DLBCL). For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “therapeutic agent” refers to any agent or material that has a beneficial effect on the mammalian recipient.

The invention will now be illustrated by the following non-limiting Examples.

### Example 1. Isoform-Specific Knockdown of Long and Intermediate Prolactin Receptors Interferes with Evolution of B-Cell Neoplasms

Prolactin (PRL) may be elevated in B-cell mediated lymphoproliferative diseases and may promote B-cell survival. However, whether PRL or PRL receptors drive the initiation, establishment, and sustenance of B-cell malignancies is unknown. We measured changes in B cells after knocking down the pro-proliferative, anti-apoptotic long isoform of the PRL receptor (LFPRLR) in vivo in systemic lupus erythematosus (SLE)- and B-cell lymphoma-prone mouse models, and the LFPRLR plus intermediate isoforms (LF/IFPRLR) in vitro in malignant human B cells. As described herein, to knockdown LF/IFPRLRs without suppressing expression of the counteractive short PRLR isoforms (SFPRLRs), splice-modulating DNA oligomers were employed. In SLE-prone mice, LFPRLR knockdown reduced numbers and proliferation of B-cell subsets and lowered the risk of B-cell transformation by downregulating expression of activation-induced cytidine deaminase. LFPRLR knockdown in lymphoma-prone mice reduced B-cell numbers and their expression of BCL2. In overt human B-cell malignancies, LF/IFPRLR knockdown reduced B-cell viability and their MYC and BCL2 expression. Unlike their normal counterparts which produced no autocrine PRL and expressed SFPRLRs, malignant human B cells expressed autocrine PRL and often no SFPRLRs. Isoform-specific knockdown of the LF/IFPRLR was not toxic to mice or to normal human immune cells, suggesting it is a safe approach to block the evolution of B-cell malignancies.

### Introduction

B-cell malignancies, such as diffuse large B-cell lymphoma (DLBCL) and acute lymphoblastic leukemia (ALL), have widely divergent outcomes^{1,2}. To identify early interventions for high-risk B-cell malignancies, we are delineating the mechanisms underlying the evolution of these neoplasms: B-cell transformation (Stage 1), overt neoplasm establishment (Stage 2), and sustenance of overt malignancy (Stage 3).

Because the initiation of B-cell malignancies is >3-fold more frequent in patients with the B cell-mediated autoimmune disease, systemic lupus erythematosus (SLE), than the healthy population³⁻⁸, SLE-prone mouse models represent robust biological systems to identify signaling pathways that elevate the risk of B-cell transformation. Similarly, because B-cell cancers often clonally evolve from pre-malignant and indolent B-cell clones⁹⁻¹¹, transgenic mice prone to developing B-cell neoplasms are robust models for investigating the mechanisms that promote the establishment of overt B-cell malignancies. For analysis of sustenance of overt B-cell malignancies, we have used cell lines and samples derived from patients with these neoplasms in this Example.

Prolactin (PRL), a sex hormone, classically recognized for its crucial role in lactation but produced by both sexes in response to acute and chronic stress¹², may drive pathological processes including tumorigenesis^{13,14}. It was postulated herein that PRL promotes the evolution of B-cell malignancies because it (1) may promote the survival of autoreactive B cells¹⁵ and may be associated with the exacerbation of SLE^{16,17}, a disease that increases the risk of B-cell transformation (2) may enhance the survival of normal and pathogenic mouse B cells and induce their expression of the proto-oncogenes MYC and BCL2 in vitro¹⁸⁻²¹ and (3) may complex with the immunoglobulin G (IgG) heavy chain to promote proliferation of malignant B cells in chronic lymphocytic leukemia (CLL), a malignancy associated with autoimmune manifestations²².

Murine and human PRL receptors (PRLRs) are cytokine receptors with long (LF) and short (SF) isoforms, generated by alternative splicing²³. Humans have an additional intermediate splice isoform (IF)²⁴, at least in pathological tissues. These different PRLRs have identical extracellular and transmembrane domains but differ in their intracellular domains such that they initiate different signals. A major signaling difference between the two categories of receptor is that the LFPRLR can activate the Janus kinase/signal transducer and activation of transcription (Jak/STAT) pathway, whereas the shorter cytoplasmic domains of the SFPRLRs cannot engage STAT¹³.

Whether PRL, working through specific PRLR isoforms, drives the genesis (Stage 1), establishment (Stage 2), and maintenance (Stage 3) of overt B-cell malignancies is unknown. It was hypothesized herein that, PRL, by signaling through LF/IFPRLR, enhances the risk of clonal evolution of autoimmune and pre-malignant B cells and sustains overt B-cell malignancies. To test this, we measured the effects of knocking down expression of the LFPRLR on B cells in vivo in mouse models of SLE²⁷ (Stage 1) and DLBCL²⁸ (Stage 2), and of the LF/IFPRLR in vitro in neoplastic human B-cell lines (Stage 3).

For isoform-specific knockdown of LFPRLR in mice and LF/IFPRLR in human cells, we employed an innovative and tractable approach involving, e.g., 25mer splice-modulating DNA morpholino oligomers (SMOs), termed here ‘LFPRLR SMO’, that selectively prevent splicing to produce the LFPRLR in mice and LF and IF PRLRs in humans. More specifically, the SMOs prevent the inclusion of exon 10 during pre-mRNA splicing, that encodes an intracellular domain of the PRLR required for binding STAT proteins. The sequence of LFPRLR SMO is different for mouse and human²⁹. The SMOs are highly stable, non-immunogenic DNA oligomers, e.g., linked to an octaguanidine dendrimer that ensures effective whole-body uptake. They are administered subcutaneously and have been found to be efficacious and non-toxic in healthy mice and syngeneic and human xenograft models of breast cancer^{29,30}.

It was found herein that signaling of PRL through the LF/IFPRLR raises the risk of B-cell transformation in SLE-prone mice, increases the survival of premalignant B cells in DLBCL-prone mice, and enhances the survival of established human B-cell malignancies. High expression of PRL in tumors associates with poor clinical prognosis in patients with B-cell malignancies. We identify blocking synthesis of the LF/IFPRLR using LFPRLR SMO as an isoform-specific and clinically attractive strategy that merits further investigation as a potential treatment for autoimmune and malignant B cell-mediated lymphoproliferative diseases.

### Results

### Knockdown of LFPRLR Reduces Splenic B-Cell Subsets in SLE-Prone Mice

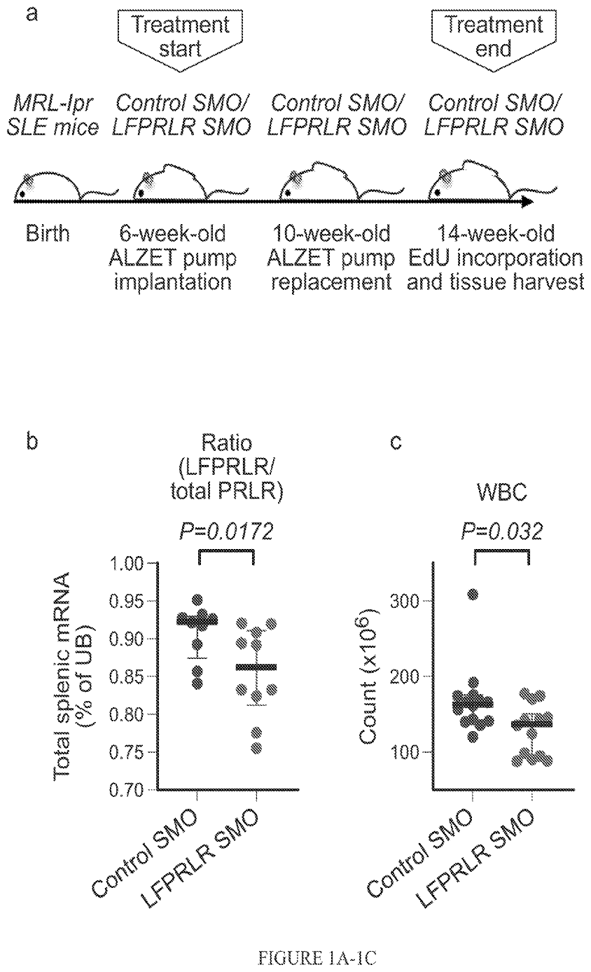

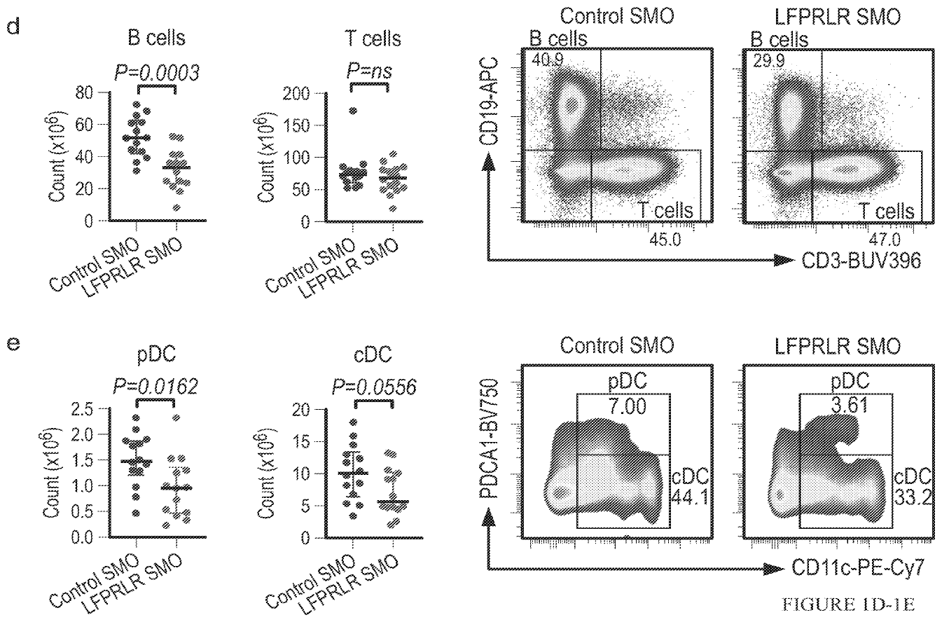

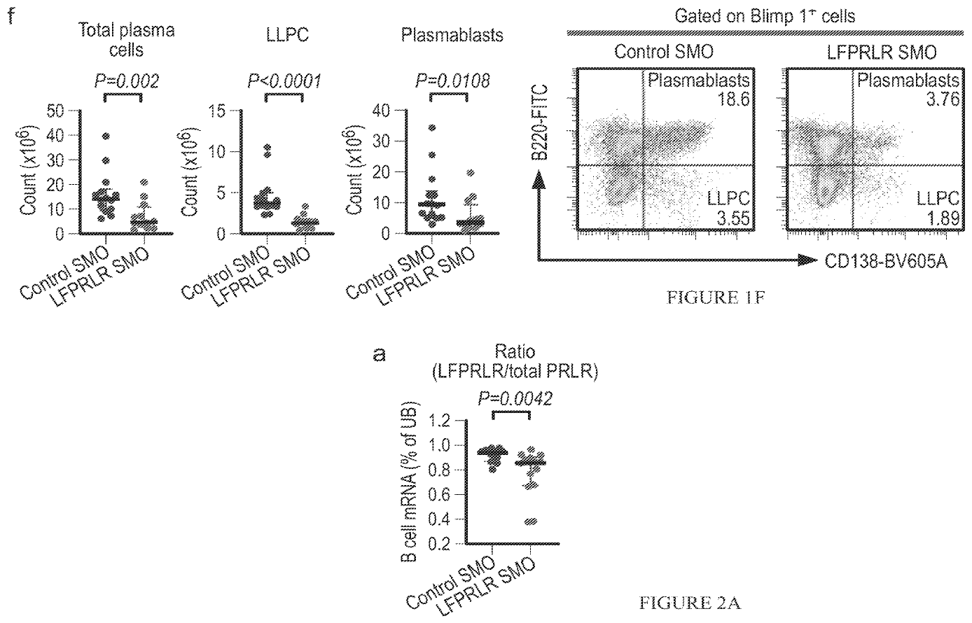

To investigate whether the PRL-LFPRLR axis raises the risk of B-cell transformation, SLE-prone MRL-lpr mice treated with either control SMO or LFPRLR SMO were compared. Among SLE-prone models, MRL-lpr mice were chosen because they accumulate genetic lesions indicative of B-cell transformation³¹ and exhibit the spontaneous lymphoproliferation mutation Fas^{lpr27} that might be implicated in lymphoma pathogenesis^{32,33}. Female MRL-lpr mice succumb to SLE between 17-22 weeks postnatally²⁷. Therefore, female mice were treated with control SMO or LFPRLR SMO from the age of 6 weeks until 14 weeks when we quantified expression of molecular indicators known to raise the risk of B-cell transformation (FIG. 1a). Using qPCR, it was confirmed that treatment with LFPRLR SMO downregulated the LFPRLR: total PRLR ratio in splenic white blood cells (WBCs) (FIG. 1b).

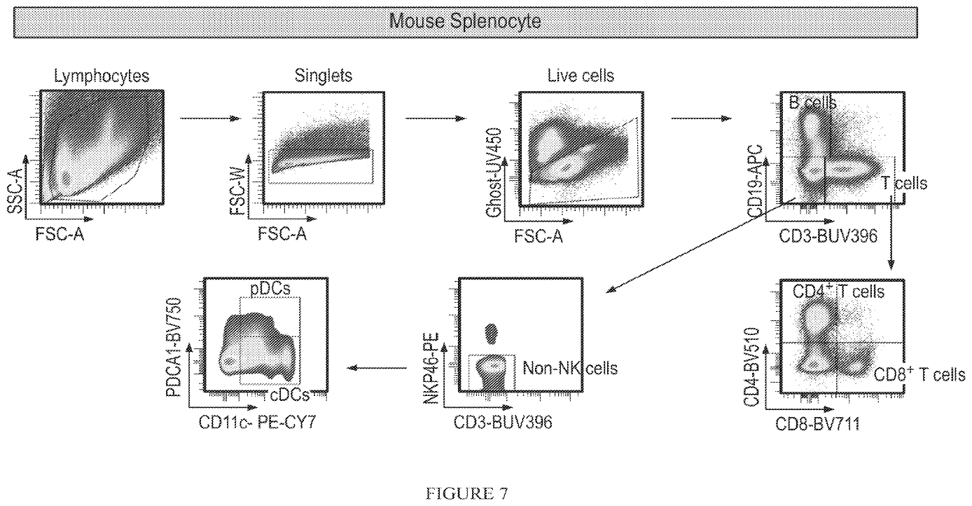

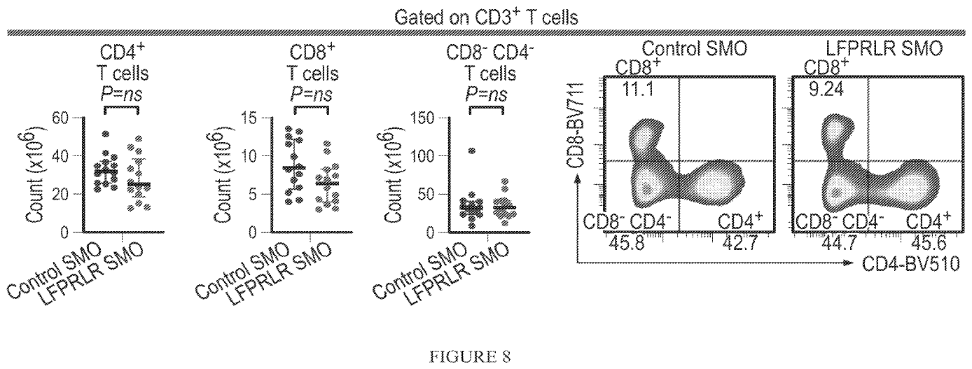

Because splenic T-, B-, and dendritic cells (DCs) drive SLE pathology³⁴, changes in these WBCs were examined after LFPRLR knockdown by flow cytometry (FIG. 7). WBC, B-cell, plasmacytoid (pDC), and conventional DC (cDC) counts were significantly reduced after LFPRLR knockdown, whereas counts of T cells and T-cell subsets (CD4⁺, CD8⁺, and pathologic CD4⁻CD8⁻ SLE T cells) remained unaltered (FIGS. 1c-e, 8).

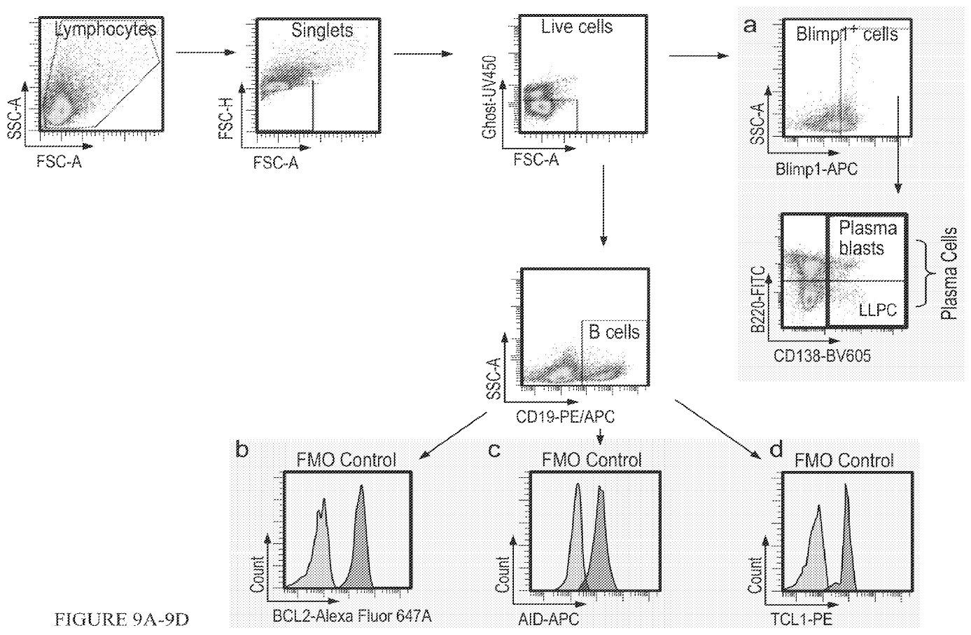

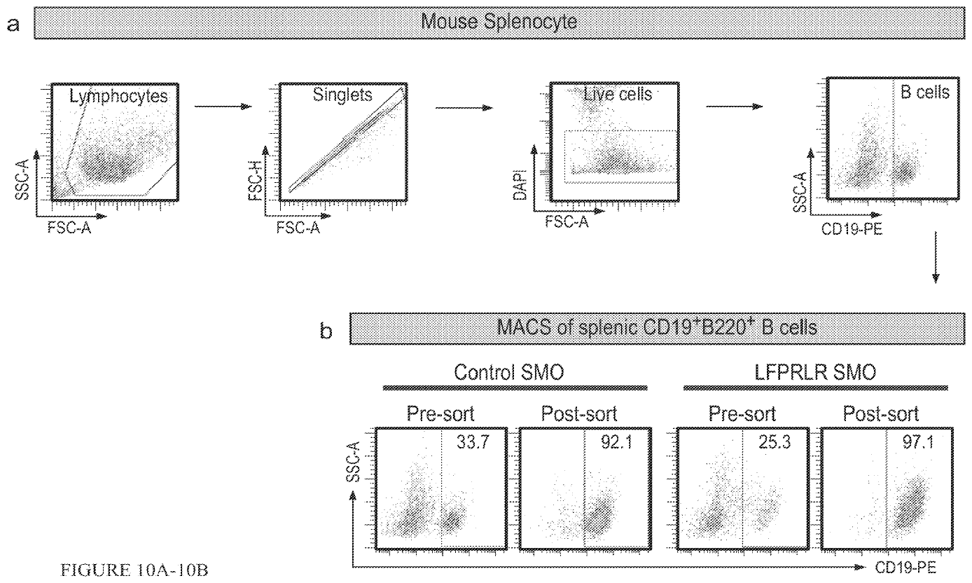

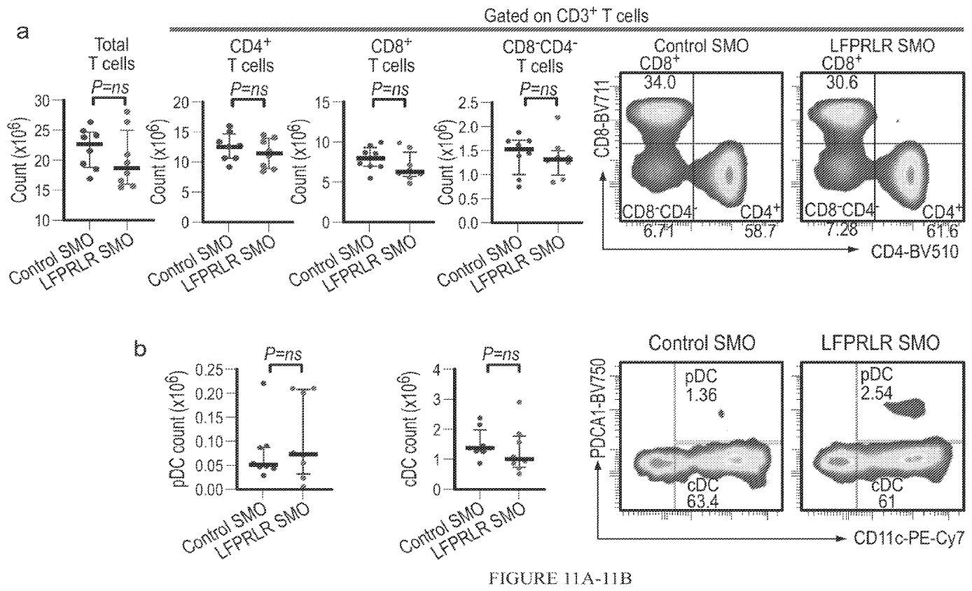

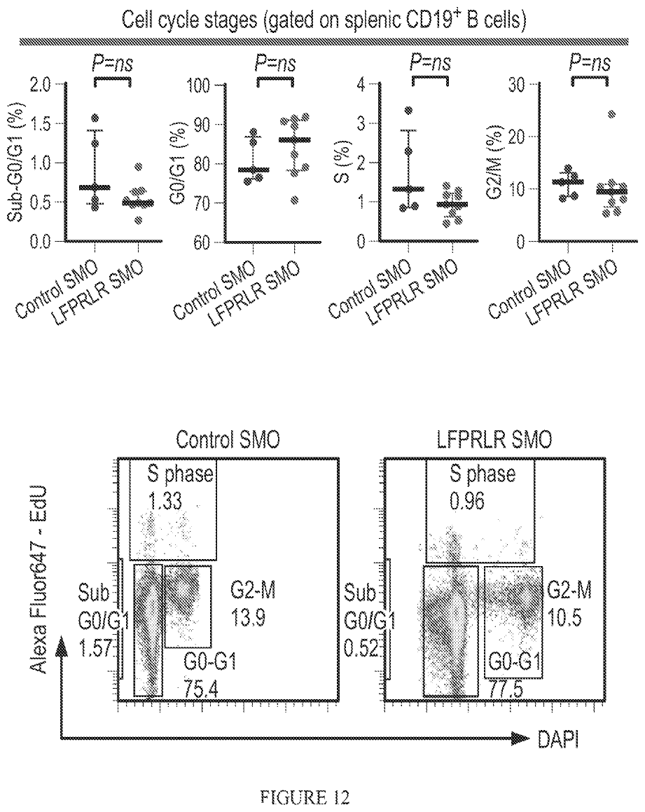

B-cell pathology in SLE is largely driven by autoantibody-producing Blimp1⁺CD138⁺B220⁺ short-lived plasmablasts and Blimp1⁺CD138⁺B220⁺ long-lived plasma cells (LLPCs)³⁵. B cells mature into plasma cells with help from DCs³⁶⁻⁴². Reductions in B cells and DCs after LFPRLR knockdown suggested that treatment with LFPRLR SMO may affect plasma cell subsets. Plasmablast and LLPC numbers were significantly reduced after LFPRLR knockdown (FIGS. 1f, 9a). We conclude that LFPRLR knockdown reduces numbers of splenic B-cell subsets in SLE-prone mice.

Knockdown of LFPRLR in SLE-Prone Mice Decreases Factors Associated with the Risk of Lymphoma Initiation

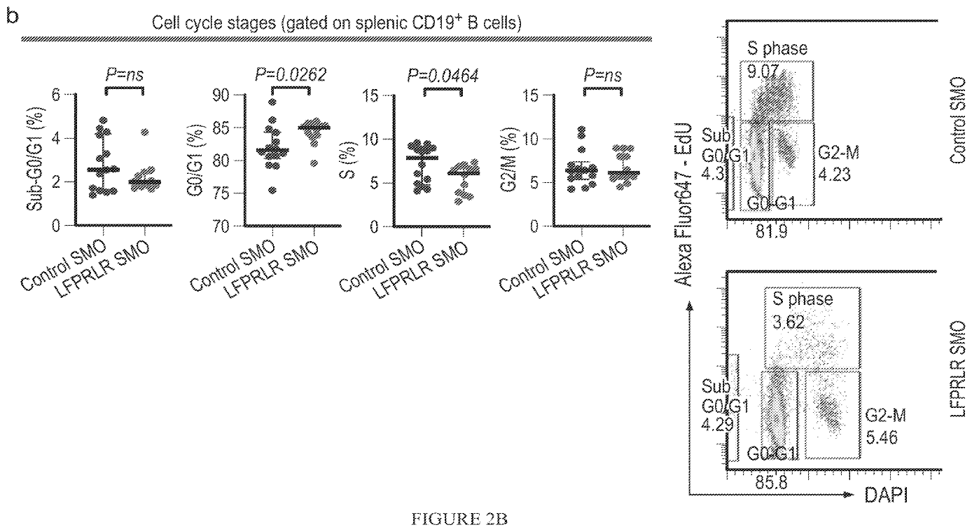

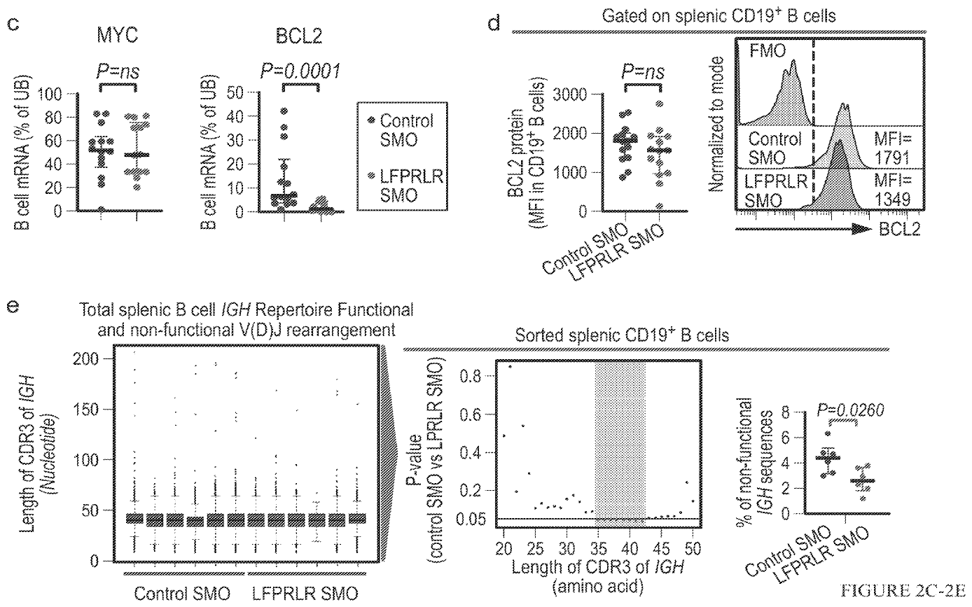

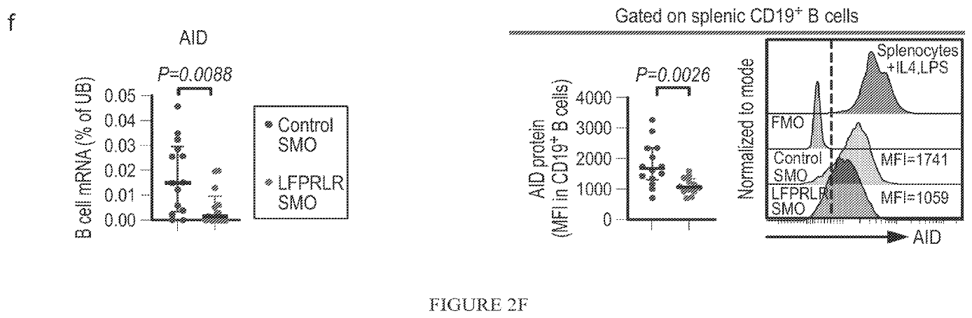

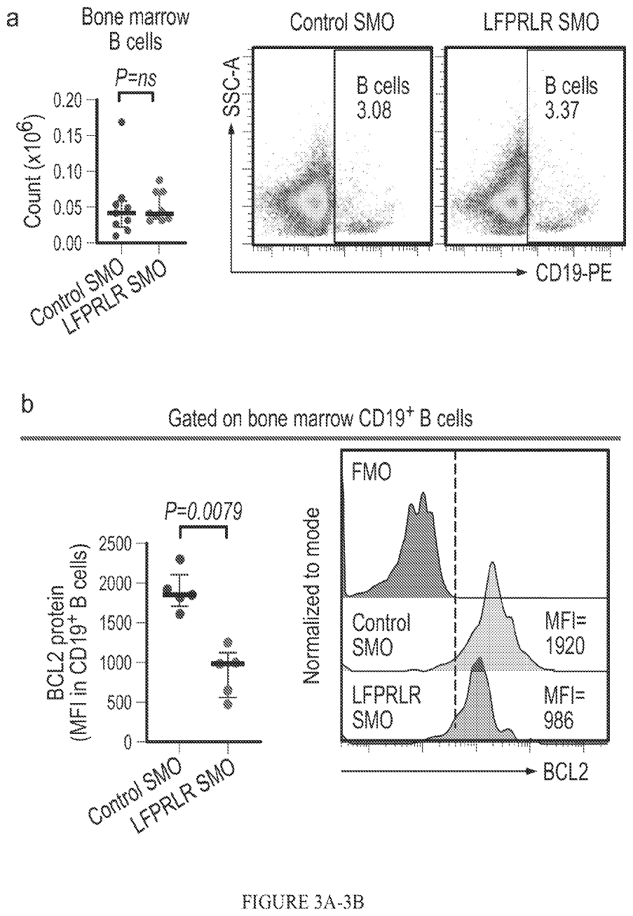

B-cell turnover and composition of the mature B-cell repertoire were examined in SLE-prone mice post LFPRLR knockdown. After confirming that treatment with LFPRLR SMO reduces the LFPRLR: total PRLR ratio in magnetically sorted splenic B cells (FIG. 10) by qPCR (FIG. 2a), B-cell proliferation was measured (FIG. 2b). LFPRLR knockdown reduced the percentage of B cells in S phase while concomitantly increasing the proportion of G0/G1 B cells. LFPRLR knockdown did not increase the frequency of pre-apoptotic sub-G0/G1 B cells, concordant with Fas dysfunctionality in MRL-lpr mice that prevents the death of arrested B cells.

Because PRL may induce the expression of MYC and BCL2 in lymphocytes¹⁸⁻²¹, the expression of these proto-oncogenes was examined in splenic B cells after LFPRLR knockdown. We found no changes in MYC, significantly reduced BCL2 mRNA, but no changes in BCL2 protein (FIGS. 2c-d, 9b). Since BCL2 protein levels affect survival rather than proliferation, no change in BCL2 protein is concordant with the reduced B-cell cycling after LFPRLR knockdown, a process that is known to be unaffected by changes in BCL2 expression in B cells⁴³. However, the significant reduction in BCL2 mRNA suggests the possibility of longer-term downregulation of BCL2 protein by LFPRLR knockdown.

Next, we investigated whether LFPRLR induced indicators of evolution of autoimmune B cells into malignancies by comparing the splenic B-cell repertoire of SLE-prone mice treated with control SMO (n=6) or LFPRLR SMO (n=6) using immunoglobulin heavy chain (IGH) sequencing. A healthy B-cell repertoire has very few deleterious, potentially polyreactive B-cell clones with IGH complementary determining region 3 (CDR3) of lengths ≥20 amino acids (≥60 nucleotides)⁴⁴⁻⁴⁷ and B cells carrying non-functional IGH rearrangements with an increased propensity towards malignant transformation¹¹. Although the B-cell repertoire was normally distributed after LFPRLR knockdown, frequencies of potentially abnormal B cells with long CDR3s ranging from 35-42 amino acids, and those with non-functional IGH rearrangements were significantly reduced in LFPRLR SMO-treated SLE-prone mice (FIG. 2e).

B-cell malignancies evolve from hyperactivation of, and off-target genomic alterations induced by, the B-cell enzyme, activation induced cytidine deaminase (AID)^{11,48}. AID diversifies the B-cell repertoire through somatic hypermutation (SHM) and class switch recombination (CSR)⁴⁹. It was hypothesized herein that PRL-LFPRLR signaling may induce AID expression because (1) estradiol, which promotes AID transcription and accelerates malignant transformation of B-lymphocytes in MRL-lpr mice⁵⁰ could, in part, mediate this process via stimulation of PRL secretion, and (2) a reduction in both DCs that mediate CSR, and LLPC that result from class-switched B cells in LFPRLR SMO-treated SLE-prone mice suggested that PRL-LFPRLR signaling may regulate AID expression. Expression of AID mRNA and protein was significantly reduced in splenic B cells after LFPRLR knockdown (FIGS. 2f, 9c). Hence, PRL signaling through LFPRLR may prime B cells for acquisition of oncogenic mutations by promoting AID expression. Hence, the PRL-LFPRLR axis allows survival of deleterious B cells, thereby increasing the risk of B-cell lymphoma initiation (Stage 1).

### Knockdown of LFPRLR Impacts Early B-Lymphopoiesis in SLE-Prone Mice

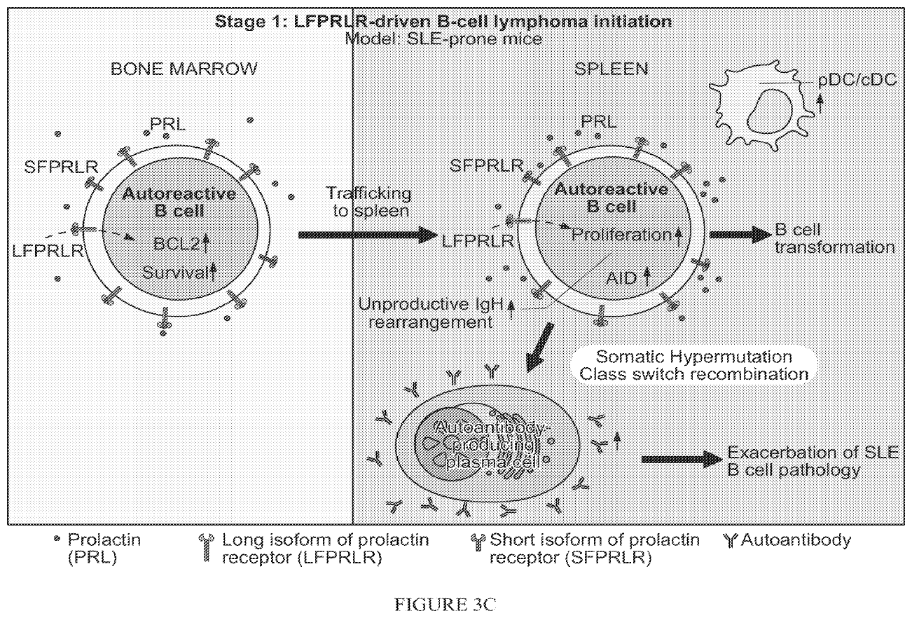

We determined the extent to which reduced splenic B cells after LFPRLR knockdown in SLE-prone mice resulted from changes in early B-lymphopoiesis. Although bone marrow B-cell counts were unchanged after LFPRLR knockdown, their expression of BCL2 was strikingly reduced (FIGS. 3a-b). Because BCL2 is critical for survival of immature B cells⁵¹, including autoreactive B cells^{45,52,53}, we conclude that the LFPRLR likely contributes to the inappropriate survival and retention of abnormal, early B cells in SLE-prone mice.

Overall, LFPRLR drives inappropriate early and mature B-lymphopoiesis in SLE-prone mice; the former mediated via induction of BCL2 expression and the latter by increasing DC numbers, which can in turn promote mature B-cell cycling, AID expression, and plasma cell generation. Consequently, deleterious B cells with non-productive IGH rearrangements and long CDR3s are retained in the repertoire, thereby raising the risk of lymphomagenesis (FIG. 3c).

Knockdown of LFPRLR in DLBCL-Prone Mice Suppresses Factors that can Drive Overt B-Cell Lymphomagenesis

To determine whether the PRL-LFPRLR axis elevates the risk of establishment of overt B-cell malignancies from pre-malignant B cells, i.e., Stage 2 of evolution of a B-cell malignancy, B-cell pathology in DLBCL-prone CD79b-TCL1-tg mice²⁸, treated with either control SMO or LFPRLR SMO, were compared. Mice hemizygous for the TCL1 transgene can develop DLBCL beginning at 4 months of age. However, most of the mice die from lymphoma when they are 7-13 months old. Mild lymphocytosis and increased splenic B-cell proliferation is observed in 1-3-month-old pre-malignant TCL1-tg mice compared to their healthy counterparts²⁸.

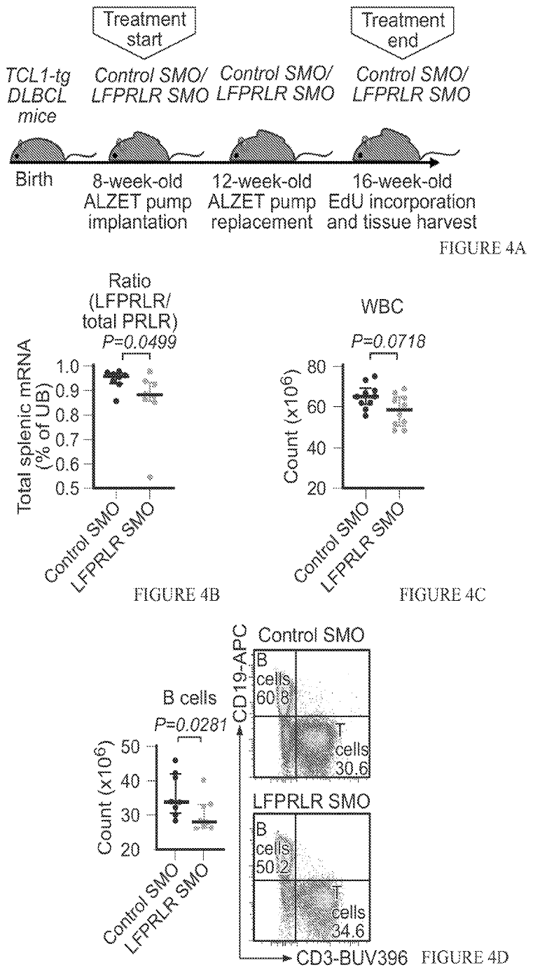

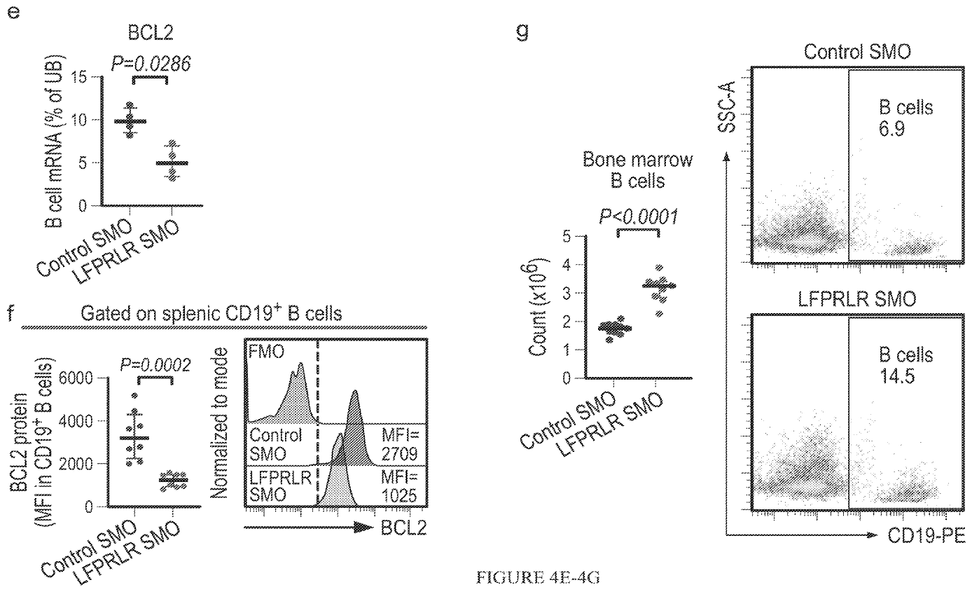

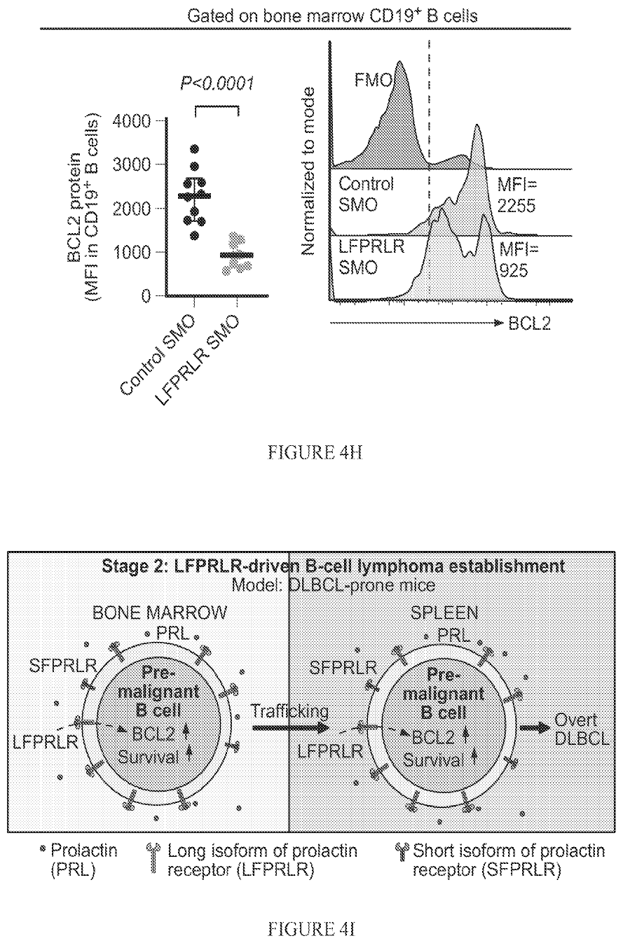

To examine the role of LFPRLR in raising the risk of overt lymphoma development from pre-malignant B cells, 8-week-old TCL1-tg mice were treated with the SMOs until 16-weeks-old, which, in most mice is before the development of overt DLBCL (FIG. 4a). We confirmed LFPRLR knockdown in total splenic WBCs by qPCR (FIG. 4b) and observed a trend towards reduced splenic WBC after LFPRLR knockdown (FIG. 4c). Among splenic WBC subsets, LFPRLR knockdown significantly reduced B-cell counts but not counts of total T cells, T-cell subsets, or DC subsets (FIGS. 4d, 11). Hence, abrogation of synthesis of the LFPRLR specifically impacts pre-malignant B cells and provides the rationale for interrogating the role of LFPRLR in Stage 2 of B-cell lymphomagenesis.

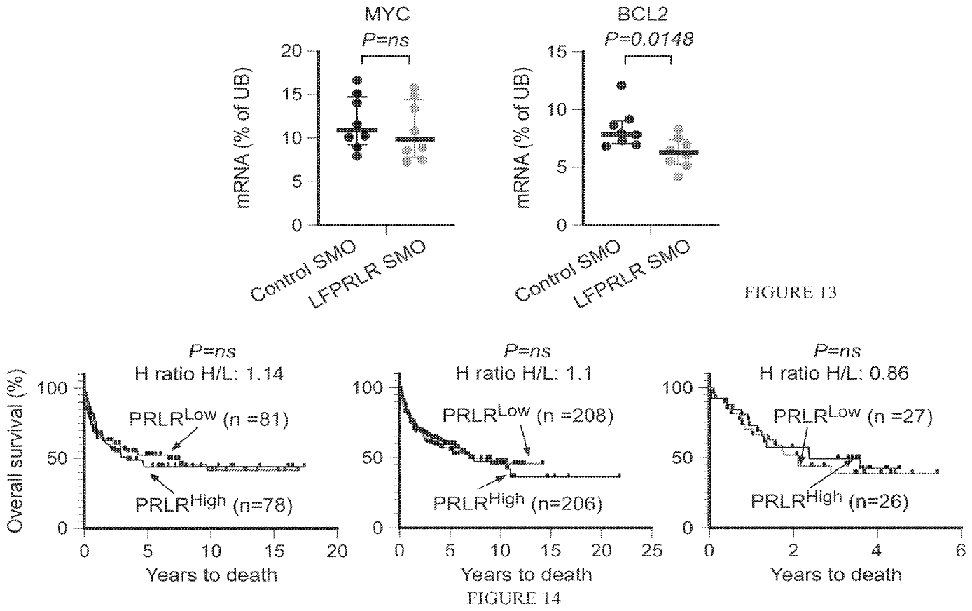

B-cell cycling was unaffected after LFPRLR knockdown in TCL1-tg mice (FIG. 12). To examine whether LFPRLR knockdown modulated expression of MYC and BCL2 mRNA, we initially used splenic WBCs because we could sort only a limited number of B cells even after pooling samples from −3-4 mice per group. MYC expression was unchanged but that of BCL2 was significantly downregulated after LFPRLR knockdown (FIG. 13). With the few sorted pooled splenic B cells, the downregulation of BCL2 mRNA and protein in pre-malignant B cells were confirmed after LFPRLR knockdown (FIGS. 4e-f).

In contrast to SLE-prone mice, bone marrow B-cell counts in DLBCL-prone mice were significantly increased after LFPRLR knockdown. However, their expression of BCL2 was again significantly reduced (FIGS. 4g-h). Together with reduced splenic B-cell numbers (FIG. 4d), this suggests that LFPRLR knockdown impairs the dissemination of the pre-malignant bone marrow B cells into the periphery.

Overall, the PRL-LFPRLR axis promotes BCL2 expression in early and mature pre-malignant B cells, thereby raising the chance of overt B-lymphomagenesis (FIG. 4i).

### PRL-LFPRLR Signaling Maintains Overt Human B-Cell Malignancies

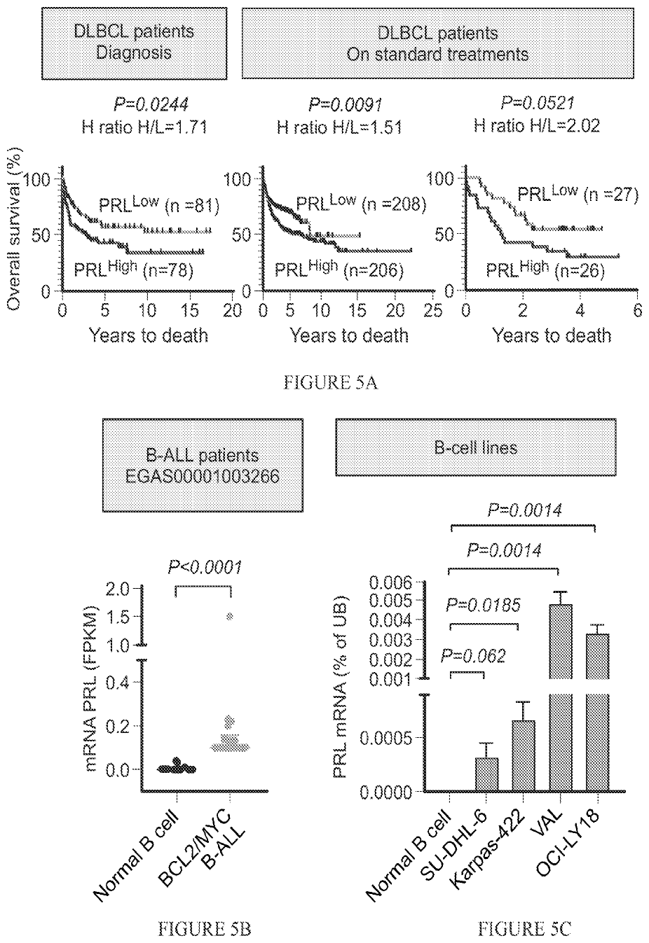

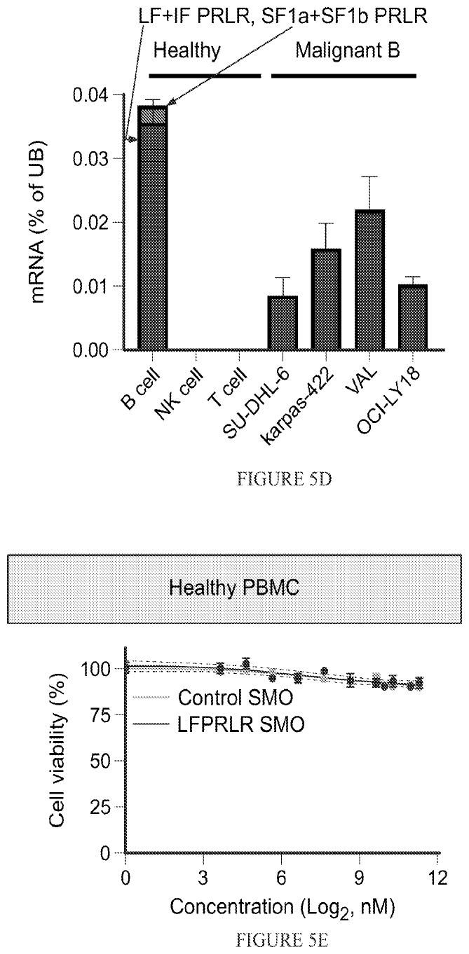

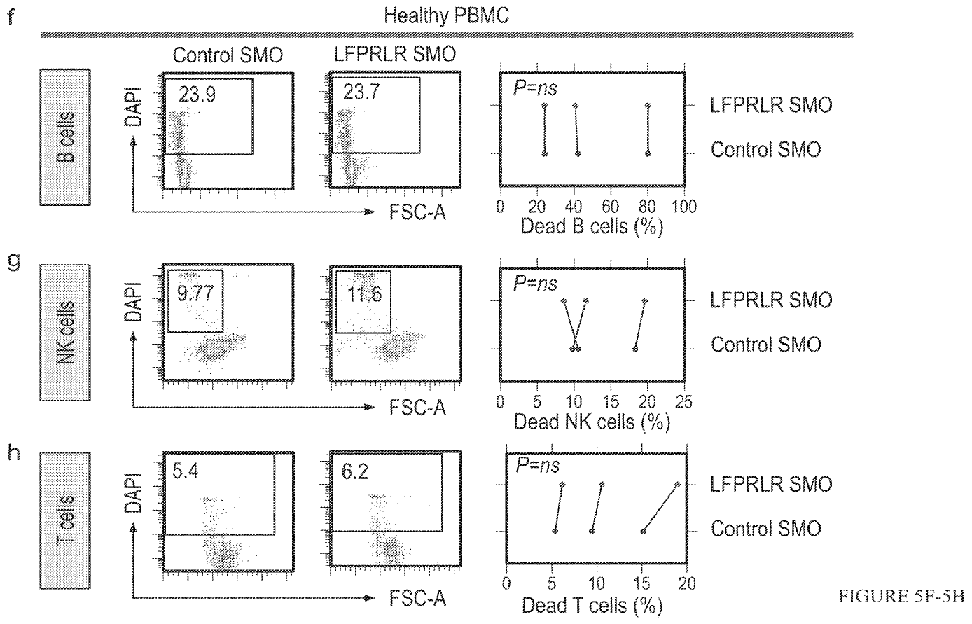

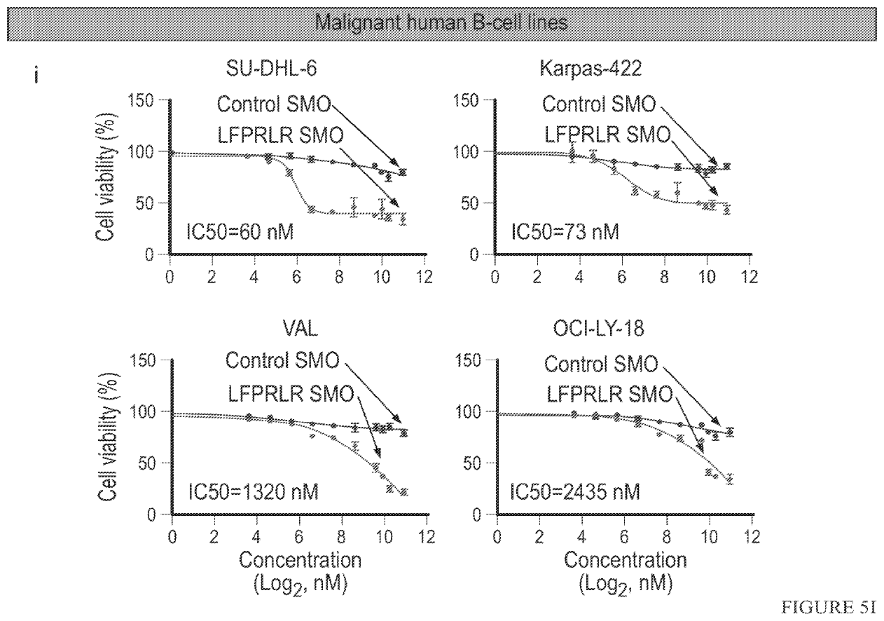

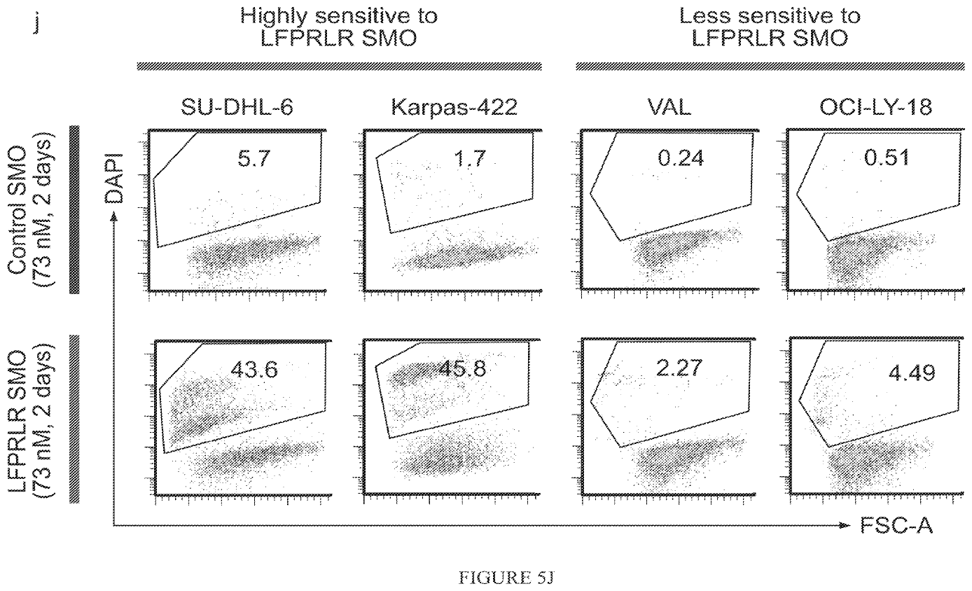

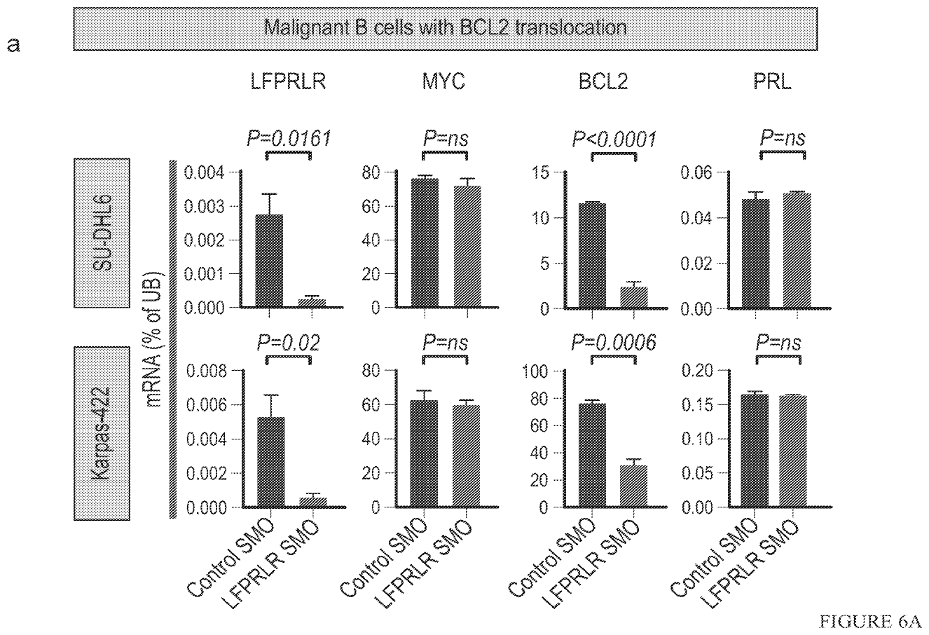

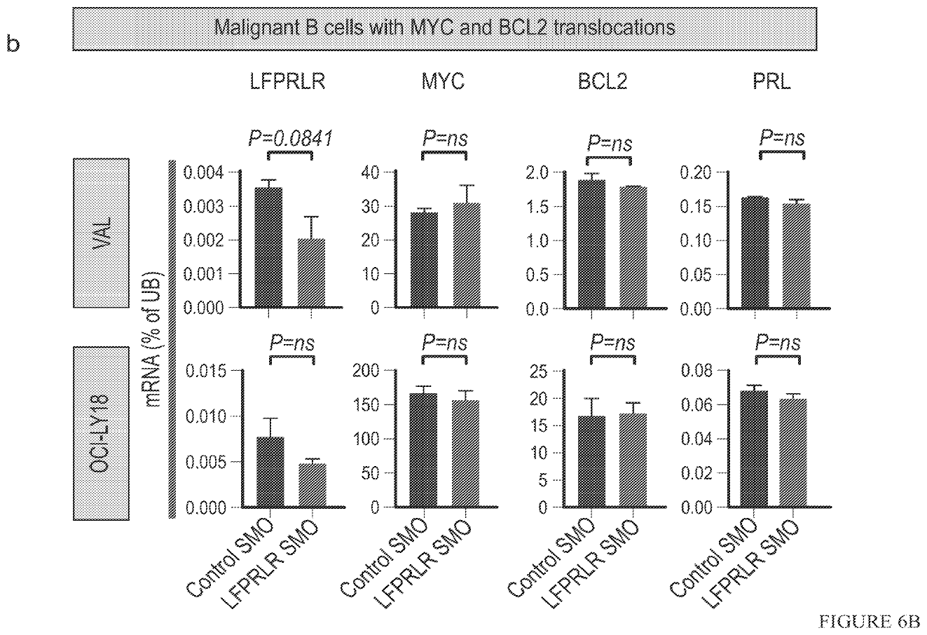

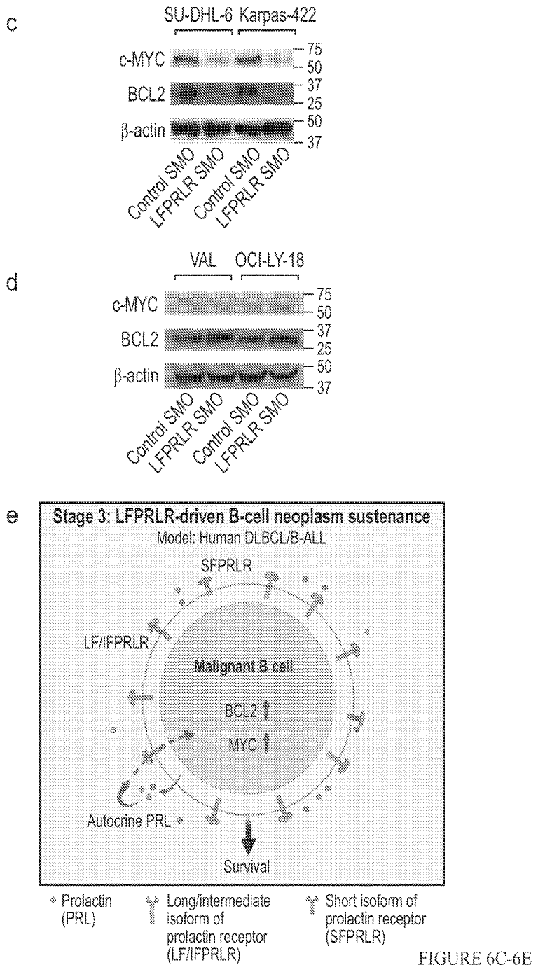

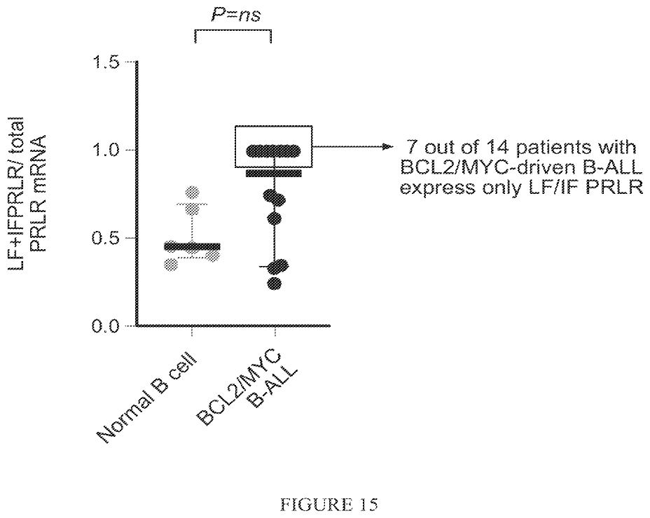

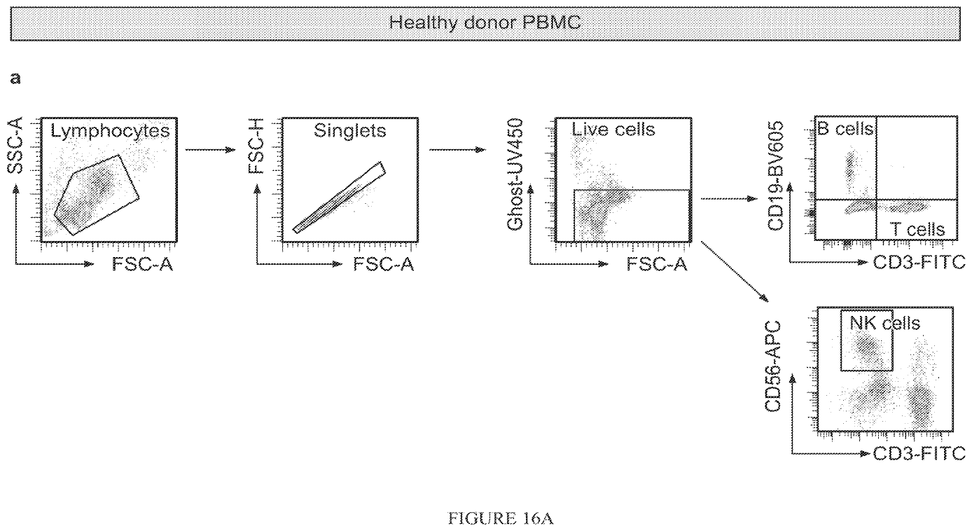

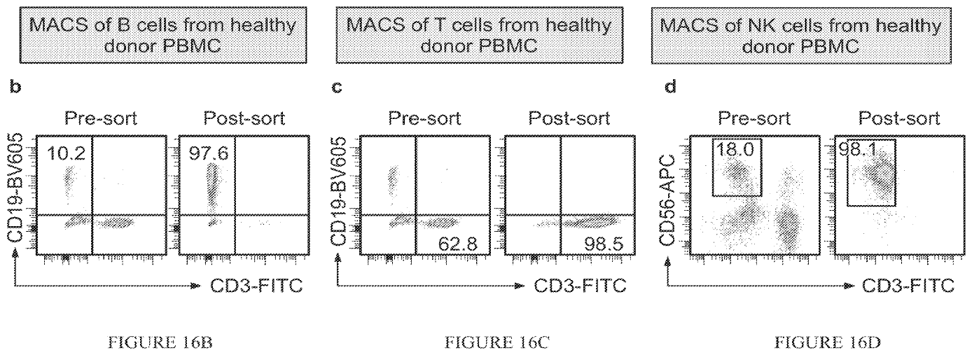

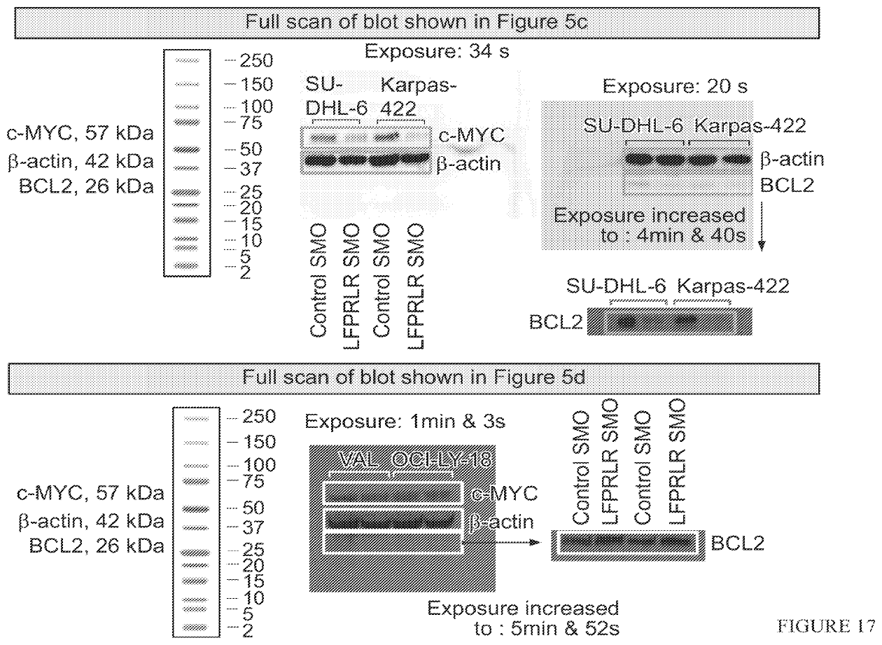

We determined whether expression of autocrine/paracrine PRL in tumor samples from patients with DLBCL and/or Burkitt's lymphoma (BL)⁵⁴⁻⁵⁶ was indicative of clinical outcome. Higher-than-median expression of PRL transcript (PRL^{high}) significantly associated with lower overall survival in patients with DLBCL/BL, both at diagnosis⁵⁴ and after standard treatments^{55,56} (FIG. 5a). Because expression of individual PRLR isoforms in these samples was unavailable, we compared overall survival after separating patients into PRLR^{high} and PRLR^{low} groups based on their median mRNA expression of total PRLR. Total PRLR levels did not correlate with survival of DLBCL/BL patients (FIG. 14), suggesting either that local production of autocrine PRL was more important than levels of PRLR expression and/or that some tumors still express some level of SFPRLRs.

Next, we investigated whether B-lymphoblasts in ALL patients with poorly prognostic MYC/BCL2-driven B-ALL⁵⁷ express more autocrine PRL than their healthy counterparts. Unlike normal B cells, B cells in B-ALL patients produced autocrine PRL (FIG. 5b). While normal B cells expressed both LF/IF and SFs, 50% of the patients with MYC/BCL2-driven B-ALL expressed only the LF/IFPRLR (FIG. 15). Consistent with these findings, unlike normal B cells of healthy donors, MYC/BCL2-driven B-ALL (VAL) and DLBCL (Karpas-422, SU-DHL-6, OCI-Ly18) human cell lines expressed autocrine PRL and only the LF/IFPRLR (FIGS. 5c-d). Thus, the upregulation of PRL-LF/IFPRLR signaling (high PRL and no SFPRLR) likely contributes to the maintenance of aggressive B-cell malignancies.

The preclinical potential of the LFPRLR SMO was confirmed by showing that LFPRLR knockdown is not toxic to healthy peripheral blood mononuclear cells (PBMCs) (FIG. 5e). Although normal B cells express more LF than SFPRLRs (FIG. 5d), SFPRLRs do not have to be present in a 1:1 ratio to inhibit the activity of LFPRLRs²⁴⁻²⁶. Moreover, the lack of production of autocrine PRL by normal B cells (FIGS. 5b-c) likely renders them insensitive to LFPRLR knockdown (FIG. 5f). Unlike B cells, most normal T and NK cells did not express the LFPRLR (FIG. 5d) and, therefore, administration of the LFPRLR SMO was not toxic to these cells (FIGS. 5g-h, 16).

Malignant B-cell survival after LFPRLR SMO treatment was measured by 3-(4,5-dimethylthiazol-2-yl)-5-(3-carboxymethoxyphenyl)-2-(4-sulfophenyl)-2H-tetrazolium (MTS) assay and flow cytometry. Because malignant B cells produce PRL that can drive their PRLR signaling, exogenous PRL was not added in experiments of this Example 1. LFPRLR knockdown killed Karpas-422 and SU-DHL-6 DLBCLs with half maximal inhibitory concentration (IC50) at 60-70 nM of LFPRLR SMO; however, IC50s for the VAL B-ALL and OCI-Ly18 DLBCL cell lines were 20-40-fold higher (FIGS. 5i-j). Hence, human B-cell malignancies are differentially sensitive to knockdown of the LF/IFPRLR.

### Knockdown of LFPRLR in Malignant Human B Cells Reduces BCL2 and MYC Expression

We investigated why the Karpas-422 and SU-DHL-6 cell lines were sensitive to LF/IFPRLR knockdown at nanomolar concentrations of LFPRLR SMO, while the VAL and OCI-Ly18 cell lines were killed only at higher concentrations (FIGS. 5i-j). We excluded the hypothesis that VAL and OCI-Ly-18 did not express the LF/IFPRLR (FIG. 5d), suggesting instead that differential sensitivity might be due to dysregulation of splicing factors required for optimal functioning of the LFPRLR SMO in these lines.

Splicing dysregulation is often caused by constitutive overexpression of MYC and BCL2⁵⁸, two important downstream targets of PRL signaling¹⁸⁻²¹, that also drive sustenance of B-cell malignancies. A striking difference between the LFPRLR SMO-sensitive SU-DHL-6 and Karpas-422 and the less sensitive VAL and OCI-Ly18 lines is the constitutive expression of both MYC and BCL2 from chromosomal translocations in VAL and OCI-Ly18. Therefore, we determined whether an inability to downregulate MYC and/or BCL2 in VAL and OCI-Ly18 reduced their sensitivity to low concentrations of LFPRLR SMO. Treatment with a low concentration (73 nM) of LFPRLR SMO reduced MYC protein and BCL2 mRNA and protein in the sensitive SU-DHL-6 and Karpas-422 lines, but such reduction was absent in the VAL and OCI-Ly18 lines (FIGS. 6a-d, 17). Overall, B-cell malignancies rely on PRL signaling to different extents, and the extent of this reliance is likely decided by the cell's ability to constitutively and concurrently overexpress MYC and BCL2 (FIG. 6e).

### Discussion

In this Example, the newly identified causal role of the PRL-LF/IFPRLR axis in promoting the evolution of B-cell malignancies suggests that the LF/IFPRLR may represent a therapeutic target in patients with B-cell malignancies and in those vulnerable to developing these malignancies.

The molecular mechanisms underlying the increased risk of transformation of B-cell clones in autoimmune diseases are not well understood³⁴. We find that LFPRLR not only promotes the accumulation of deleterious B cells by increasing their proliferation and their chances of being mutated by AID, but also exacerbates B-cell pathology in SLE. Thus, targeting the synthesis of the LF/IFPRLR represents an attractive strategy to lower the risk of SLE-associated B-lymphomagenesis.

The LFPRLR promotes BCL2 expression in pre-malignant B-cells, thereby underscoring its importance in driving overt B-cell lymphomagenesis. Given the limited attempts to interfere with cancer progression from a pre-malignant state to overt cancer, studies in this Example support the importance of exploring the potential of the LFPRLR SMO in preventing overt leukemogenesis/lymphomagenesis from pre-leukemic¹⁰ and indolent B cells⁹ and monoclonal B-cell lymphocytosis⁵⁹.

We find that PRL-LF/IFPRLR signaling maintains the growth and survival of established DLBCL and B-ALL, and that this maintenance is associated with the induction of MYC and BCL2. The LFPRLR SMO may thus be a non-toxic alternative to inhibit the production of the ‘difficult-to-drug’ MYC oncoprotein⁶⁰ and to block the production of BCL2. Findings in this Example showing that normal T and NK cells are insensitive to LF/IFPRLR knockdown also suggest that LFPRLR SMO may be combined with immune cell-based therapies for treating B-cell malignancies.

The LFPRLR SMO offers several advantages over other drugs that affect PRL signaling. For example, dopamine agonists that inhibit PRL production by the pituitary^{34,61} have no effect on non-pituitary autocrine/paracrine sources of PRL in the body⁶², including malignant B cells. Dopamine agonists would also abrogate beneficial PRL-SFPRLR signaling, which may mediate apoptosis of deleterious B cells. By isoform-specific targeting, the chances of off-target toxicities are reduced, such as in the liver, which primarily expresses the SFPRLRs⁶³. Traditional receptor antagonists or anti-PRLR antibodies affect all PRLR isoforms since they all have identical extracellular domains²⁰. Therefore, unlike the LFPRLR SMO, they cannot specifically target the axis governing disease evolution.

Our goal was to determine the role of signaling through the LFPRLR at different stages of B-cell malignancy evolution by examining mouse models exhibiting increased risk of initiation or enhanced progression of this disease and human data. Death from SLE in control SMO-treated Mrl-lpr mice²⁷ precludes survival studies that determine the extent to which LFPRLR knockdown blocks the initiation of B-cell lymphomas. In TCL1-tg mice, development of overt DLBCL is slow and uneven²⁸, demanding survival studies with a large number of mice and of more than a year's duration. Therefore, in both models, we instead examined how LFPRLR promotes the expression of molecular drivers of B-lymphomagenesis. Current findings in this Example strongly support a role for LFPRLR signaling at all stages of disease and justify further preclinical development of the LFPRLR SMO.

### Methods

### SMO

SMO and control oligomers linked to octaguanidine dendrimers for cell/tissue penetration in vivo, were custom synthesized (Gene Tools, Philomath, Oregon) (Table Sla). SMO sequences were designed to bind to the intron 9-exon 10 junction in mouse or human LF/IFPRLR pre-mRNA²⁹.

TABLE S1a
Splice modulating oligomer

| SMO | Sequence |
|---|---|
| Control SMO | 5′-AGACGAGATTCGATCGGAGTA-3′ |
| | (SEQ ID NO: 11) |
| mLFPRLR SMO | 5′-GCCCTTCTATTGAAACACAGATACA-3′ |
| | (SEQ ID NO: 2) |
| HLFPRLR SMO | 5′-GCCCTTCTATTAAAACACAGACACA-3′ |
| | (SEQ ID NO: 1) |

### Animal Models

Animal studies were conducted in compliance with Institutional Animal Care and Use Committees at City of Hope and University of California, Riverside. 6-week-old mice female SLE-prone MRL-lpr mice homozygous for the Fas cell surface death receptor mutation (Fas^{lPr}) were anesthetized with isoflurane, and Alzet minipumps (Durect, Cupertino, CA) were implanted subcutaneously between the scapulae. Mice were randomly assigned to control SMO or LFPRLR SMO groups and coded by ear punch. Animals in each group were housed individually after pump implantation until wound clips were removed. Alzet pumps that delivered 100 pmoles/h/mouse of either control SMO or LFPRLR SMO were changed after 4 weeks. At week 8 of treatment, two hours before euthanasia, each animal received an intraperitoneal injection of 2.8 mg of the nucleoside analog, 5-ethynyl-2′-deoxyuridine (EdU). 8-week-old male and female DLBCL-prone TCL1-tg mice were implanted with Alzet minipumps and treated with control or LFPRLR SMO for 8 weeks, as described for MRL-lpr mice.

### Tissue Processing

Spleens were placed in Roswell Park Memorial Institute (RPMI) medium containing 40 units/ml DNAse (RPMI⁺), transferred to a 70 μM cell strainer over a new dish, and mashed with a syringe plunger. Bone marrow was flushed from femurs and tibia with RPMI⁺ using a syringe with a 25½ G needle into a 70 μM cell strainer. A syringe plunger broke up cell clumps and residual cells were washed through the strainer. After pelleting and red blood cell lysis in 1× lysis buffer (BD Biosciences), WBCs were washed in RPMI+. To ensure that all samples across different groups were treated identically, cells were resuspended in freezing solution (90% FBS/10% DMSO) and stored in liquid nitrogen until analysis.

### Cell Lines and Culture

Mycoplasma-negative human cell lines were obtained from DSMZ and cultured in RPMI 1640 with 10% fetal bovine serum, 100 U/mL Penicillin, and 100 μg/mL Streptomycin (Complete RPMI) (Invitrogen/Life technologies).

Quantitative Real-Time PCR (qPCR)

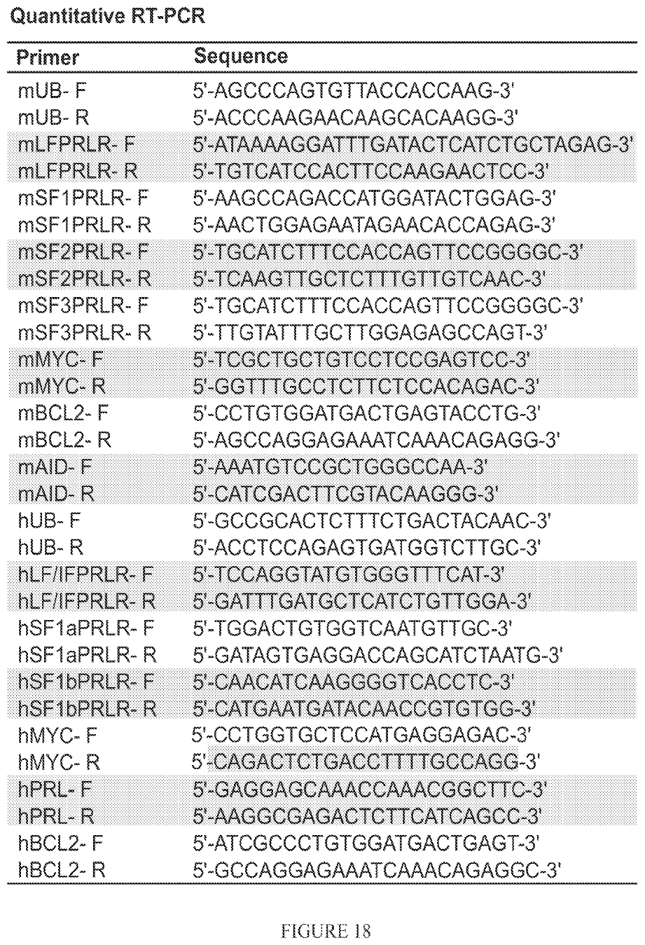

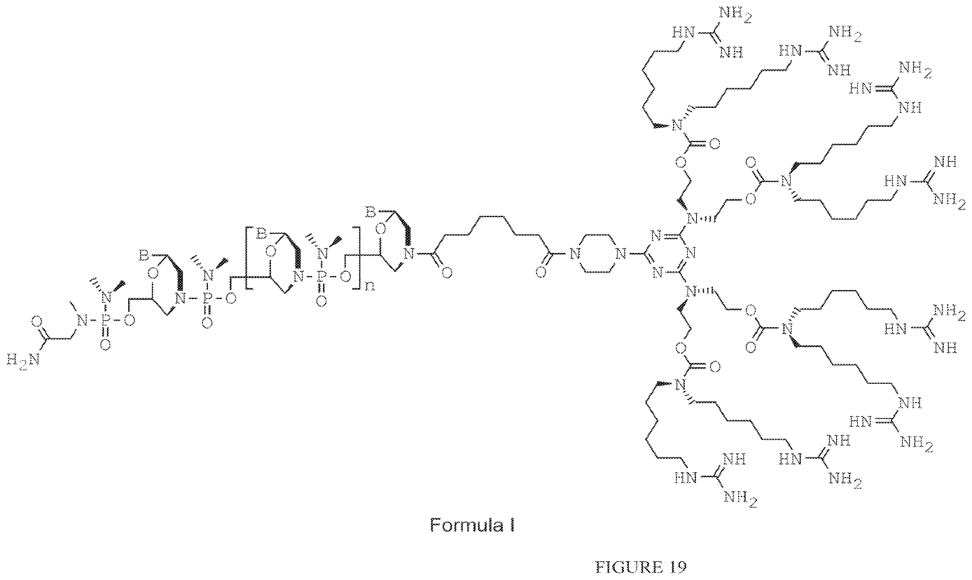

RNA was extracted using a kit (NucleoSpin® RNA Plus, MACHERY-NAGEL) according to manufacturer's instructions. cDNA was synthesized using SuperScript™ IV Reverse Transcriptase (Invitrogen). qPCR was conducted using Power SYBR Green PCR master mix (ThermoFisher Scientific) and QuantStudio 7 flex real-time PCR system (Applied Biosystems). FIG. 18 lists qPCR primers.

Expression of PRLR isoforms was represented as LF+IFPRLR/total PRLR to compare signaling outcome downstream of PRL between groups. Murine total PRLR=LF+SF3 (SF1, SF2 were not expressed in splenocytes and B cells). Human total PRLR=LF+IF+SF1a+SF1b.

### B-Cell Clonality

IGH repertoire of splenic B cells were PCR-amplified using primers for the J558 V_{H}-region gene and the Cp constant region (Table S1c), as described previously^{64,65}. Selective amplification of the J558 V_{H} family was conducted due to the increased representation of this family in both the production of autoantibodies and non-binding antibodies in MRL-lpr SLE-prone mice⁶⁶. Amplicons were subjected to next generation sequencing (NGS): TrueSeq library was prepared by adapter ligation to full-length products and sequenced on the MiSeq Illumina 2×150 bp platform. Abundances of unique nucleotide sequences were calculated and CDR3 length, spectrum, and functionality were analyzed using International Immunogenetics Information System (IMGT) HighV-QUEST. VDJTools (v1.1.7) was used for visualization. CDR3 amino acid frequencies were calculated using customized R scripts and heatmaps were generated using the pheatmap package and unsupervised hierarchical clustering using Euclidian distance. Next generation sequencing of the immunoglobulin heavy chain repertoire described in this Example is deposited in Gene Expression Omnibus under accession GSE207186.

TABLE S1c
mIGH sequencing

| Primer | Sequence |
|---|---|
| V_{H}1_F | 5′-AAGGCCACACTGACTGTAGAC-3′ |
| | (SEQ ID NO: 12) |
| Cμ_R | 5′-TGGCCACCAGATTCTTATCAG-3′ |
| | (SEQ ID NO: 13) |

### Analysis of PRLR Isoforms in B-ALL Patients

Expression of PRLR isoforms was parsed based on previous knowledge that SFs lack most of exon 10, whereas LF and IF include entire or large portions of exon 10²³. Raw paired-end fastq files of RNA-seq were mapped to human GRCh38 using STAR v.2.7.6a. Raw exon counts across the whole genome were obtained using DEXSeq⁶⁷, normalized based on variance stabilizing transformation method using R package DESeq2⁶⁸, and batch corrected using R package sva.

### Flow Cytometry

Cells were thawed in Complete RPMI and stained with fluorochrome-tagged surface antibodies and Ghost-UV450 for 30 min on ice. Using eBioscience™ Transcription Factor Staining Buffer Set, cells were fixed, permeabilized, and stained with intracellular antibodies for 30 min on ice followed by acquisition on the BD FACSymphony cytometer. Gates were set using single-stained and fluorescence minus one (FMO) controls. Data were analyzed using FlowJo10.7.1. Table S2a lists cytometry antibodies.

TABLE S2a
List of antibodies used in flow cytometry

| Antibodies | Clone ID | Specificity | Dilution | Source |
|---|---|---|---|---|
| Anti-CD19 | 1D3/CD19 | Mouse | 7:1000 | Biolegend |
| Anti- | RA3-6B2 | Mouse/Human | 5:1000 | Biolegend |
| CD45R/B220 | | | | |
| Anti-CD138 | 281-2 | Mouse | 1:100 | Biolegend |
| (Syndecan-1) | | | | |
| Anti-Blimp-1 | 5E7 | Mouse | 1:100 | Biolegend |
| Anti-AID | mAID-2 | Mouse/Human | 1:100 | eBioscience |
| Anti-BCL2 | BCL/10C4 | Mouse/Rat | 1:100 | Biolegend |
| Anti-CD3 | UCHT1 | Human | 1:100 | Biolegend |
| Anti-CD56 | 5.1H11 | Human | 1:100 | Biolegend |
| Anti-CD19 | HIB19 | Human | 1:100 | Biolegend |
| Anti-CD4 | RM4-5 | Mouse | 1:100 | Biolegend |
| Anti-CD19 | 1D3 | Mouse | 1:100 | BD |
| Anti-CD11c | N418 | Mouse | 1:100 | Biolegend |
| Anti-CD8 | 5306.7 | Mouse | 1:100 | Biolegend |
| Anti-CD3 | 17A2 | Mouse | 1:100 | BD |
| Anti-PDCA1 | 927 | Mouse | 1:100 | BD |
| Anti-NKP46 | 29A1.4 | Mouse | 1:100 | BD |
| Ghost Dye ™ UV | | | 1:100 | Tonbo |
| 450 | | | | |

### Cell Cycle

EdU incorporation was assessed using Click-iT™ Plus EdU flow cytometry. Cells were washed in 1% bovine serum albumin in phosphate buffered saline and stained for surface antigens, as above, followed by fixation, permeabilization, click-iT EdU detection, and 4′,6-diamidino-2-phenylindole (DAPI) staining. Cells were analyzed on BD FACSymphony and FlowJo10.7.1.

### Magnetic Activated Cell Sorting (MACS)

Mouse splenic B cells were enriched using CD19-microbeads (Miltenyi). Human healthy donor peripheral blood mononuclear B, T, and NK cells (PBMCs) were enriched using Miltenyi kits. Enrichment was confirmed by flow cytometry.

### Immunoblotting

Cells were lysed in RIPA buffer (Sigma) supplemented with 1% protease inhibitor ‘cocktail’ (Pierce). Proteins were separated by electrophoresis through 4-20% TGX gradient gels (BioRad) and transferred to polyvinylidene fluoride membranes (Immobilion; Millipore). Mouse and human proteins were detected by immunoblotting using antibodies (Table S2b) and western enhanced chemiluminescence (BioRad).

TABLE S2b
List of antibodies used in Immunoblotting

| Antibodies | Clone ID | Specificity | Dilution | Source |
|---|---|---|---|---|
| Anti-cMYC | D84C12 | Mouse/Human | 1:1000 | Cell Signaling |
| | | | | Technology |
| Anti-β-Actin | 8H10D10 | Mouse/Human | 1:500 | Cell Signaling |
| | | | | Technology |
| Anti-BCL2 | D17C4 | Mouse/Human | 1:1000 | Cell Signaling |
| | | | | Technology |

### MTS Assay

One thousand cells were seeded per well in 96-well plates and incubated in Complete RPMI containing different concentrations of control or LFPRLR SMO (0, 5, 25, 125, 250, 500, 750, 1000, 1500, 2000 nM) at 37° C. for 48 h. Twenty μl of MTS (Promega) was added to 100 μl of media containing cells and incubated for 3 h. Absorbance was measured at 490 nm with TECAN Infinite M1000 Pro microplate reader.

### Statistics

Exact p-values are provided if significant (P<0.05) or trending towards significance (0.05<P<0.1). Statistical tests were two-tailed. Pairwise comparisons between mouse cohorts were performed using the Mann-Whitney U test. For Kaplan-Meier survival analyses, p-values were calculated using a log-rank test. Female SLE-prone mice were used because SLE is more frequent in females. In experiments involving pre-malignant or malignant B cells, we represented both sexes equally because B-cell malignancies develop at the same frequencies in both males and females and malignant B cells from males and females had similar PRL and PRLR isoform expression. qPCR samples were run in 3 technical replicates. Experiments involving cell lines were repeated 3 times for reproducibility.

Abbreviations: DNA, deoxyribonucleic acid; mRNA, messenger ribonucleic acid; PRL, prolactin; PRLR, prolactin receptor; LFPRLR, long isoform of PRLR; IFPRLR, intermediate isoform of PRLR; SFPRLR, short isoform of PRLR; ALL, acute lymphoblastic leukemia; DLBCL, diffuse large B-cell lymphoma; SLE, systemic lupus erythematosus; MYC, MYC proto-oncogene, BHLH transcription factor; BCL2, B-cell lymphoma 2; Jak, Janus Kinase; STAT, signal transducer and activator of transcription; AID, activation induced cytidine deaminase; TCL1, T-cell leukemia/lymphoma protein 1; SMO, splice-modulating oligomer; FAS, fas cell surface death receptor; CDR3, complementary determining region 3; IGH, immunoglobulin heavy chain; V_{H}, variable region of the IGH; PBMC, peripheral blood mononuclear cells; WBC, white blood cell; NK, natural killer; DC, dendritic cell; OS, overall survival; CD, cluster of differentiation; mRNA, messenger RNA; IACUC, Institutional Animal Care and Use Committee; MTS, (3-(4,5-dimethylthiazol-2-yl)-5-(3-carboxymethoxyphenyl)-2-(4-sulfophenyl)-2H-tetrazolium); MFI, median fluorescence intensity; FMO, fluorescence minus one; IC50, half-maximal inhibitory concentration; MACS, magnetic activated cell sorting; DAPI, 4′,6-diamidino-2-phenylindole; CHOP, cyclophosphamide, hydroxydaunorubicin, vincristine, and prednisone; BL, Burkitt lymphoma; FPKM, fragments per kilobase of transcript per million mapped reads; JAX, Jackson Laboratories; NGS, next generation sequencing; UB, ubiquitin; EdU, 5-ethynyl-2′-deoxyuridine; DSMZ, Deutsche Sammlung von Mikroorganismen und Zellkulturen

### DOCUMENTS CITED IN EXAMPLE 1

1 Sinha, R., Nastoupil, L. & Flowers, C. R. Treatment strategies for patients with diffuse large B-cell lymphoma: past, present, and future. Blood and lymphatic cancer: targets and therapy 2012, 87 (2012).
2 Paul, S., Kantarjian, H. & Jabbour, E. J. in Mayo Clinic Proceedings. 1645-1666 (Elsevier).
3 Koff, J. L. & Flowers, C. R. B cells gone rogue: the intersection of diffuse large B cell lymphoma and autoimmune disease. Expert review of hematology 9, 553-561 (2016).
4 Tessier-Cloutier, B. et al. Cell of origin in diffuse large B-cell lymphoma in systemic lupus erythematosus: molecular and clinical factors associated with survival. Lupus science & medicine 6, e000324 (2019).
Bernatsky, S. et al. Lymphoma risk in systemic lupus: effects of disease activity versus treatment. 73, 138-142 (2014).
6 Bernatsky, S. et al. Cancer risk in childhood-onset systemic lupus. 15, 1-4 (2013).
7 Gokce, M. et al. Acute lymphoblastic leukaemia in a child with systemic lupus erythematosus. 21, 910-913 (2012).
8 Maheshwari, A. et al. Clinical and laboratory observation systemic lupus erythematosus and acute lymphocytic leukemia: An unusual case. 32, 154-156 (2011).
9 Lossos, I. S. & Gascoyne, R. D. Transformation of follicular lymphoma. Best practice & research Clinical haematology 24, 147-163 (2011).
Mori, H. et al. Chromosome translocations and covert leukemic clones are generated during normal fetal development. Proceedings of the National Academy of Sciences 99, 8242-8247 (2002).
11 Swaminathan, S. et al. Mechanisms of clonal evolution in childhood acute lymphoblastic leukemia. Nature immunology 16, 766-774 (2015).
12 Bernard, V., Young, J. & Binart, N. Prolactin—a pleiotropic factor in health and disease. Nature Reviews Endocrinology 15, 356-365 (2019).
13 Abramicheva, P. & Smirnova, O. J. B. Prolactin receptor isoforms as the basis of tissue-specific action of prolactin in the norm and pathology. 84, 329-345 (2019).
14 Ben-Jonathan, N., Liby, K., McFarland, M., Zinger, M. J. T. i. E. & Metabolism. Prolactin as an autocrine/paracrine growth factor in human cancer. 13, 245-250 (2002).
Peeva, E. & Zouali, M. Spotlight on the role of hormonal factors in the emergence of autoreactive B-lymphocytes. Immunology letters 101, 123-143 (2005).
16 Saha, S., Tieng, A., Pepeljugoski, K. P., Zandamn-Goddard, G. & Peeva, E. Prolactin, systemic lupus erythematosus, and autoreactive B cells: lessons learnt from murine models. Clinical reviews in allergy & immunology 40, 8-15 (2011).
17 Walker, S. E., Allen, S. H., Hoffman, R. W. & McMurray, R. W. Prolactin: a stimulator of disease activity in systemic lupus erythematosus. Lupus 4, 3-9 (1995).
18 Berczi, I., Nagy, E., De Toledo, S., Matusik, R. & Friesen, H. J. T. J. o. i. Pituitary hormones regulate c-myc and DNA synthesis in lymphoid tissue. J Immunol 146, 2201-2206 (1991).
19 Dominguez-Caceres, M. A. et al. Prolactin induces c-Myc expression and cell survival through activation of Src/Akt pathway in lymphoid cells. Oncogene 23, 7378-7390 (2004).
20 Flores-Fernández, R. et al. Prolactin Rescues Immature B Cells from Apoptosis-Induced BCR-Aggregation through STAT3, Bcl2ala, Bcl212, and Birc5 in Lupus-Prone MRL/lpr Mice. Cells 10, 316 (2021).
21 Kochendoerfer, S., Krishnan, N., Buckley, D. & Buckley, A. J. J. o. E. Prolactin regulation of Bcl-2 family members: increased expression of bcl-xL but not mcl-1 or bad in Nb2-T cells. J Endocrinol 178, 265-274 (2003).
22 Walker, A. M. et al. Prolactin-immunoglobulin G complexes from human serum act as costimulatory ligands causing proliferation of malignant B lymphocytes. Proceedings of the National Academy of Sciences 92, 3278-3282 (1995).
23 Trott, J., Hovey, R., Koduri, S. & Vonderhaar, B. Alternative splicing to exon 11 of human prolactin receptor gene results in multiple isoforms including a secreted prolactin-binding protein. Journal of molecular endocrinology 30, 31-48 (2003).
24 Grible, J. M. et al. The human intermediate prolactin receptor is a mammary proto-oncogene. NPJ breast cancer 7, 1-11 (2021).
Tan, D. & Walker, A. M. Short form 1b human prolactin receptor down-regulates expression of the long form. Journal of molecular endocrinology 44, 187 (2010).
26 Van Coppenolle, F. et al. Prolactin stimulates cell proliferation through a long form of prolactin receptor and K+ channel activation. Biochemical Journal 377, 569-578 (2004).
27 Andrews, B. S. et al. Spontaneous murine lupus-like syndromes. Clinical and immunopathological manifestations in several strains. The Journal of experimental medicine 148, 1198-1215 (1978).
28 Hoyer, K. K. et al. Dysregulated TCL1 promotes multiple classes of mature B cell lymphoma. Proceedings of the National Academy of Sciences 99, 14392-14397 (2002).
29 Yonezawa, T. et al. Anti-metastatic outcome of isoform-specific prolactin receptor targeting in breast cancer. Cancer Letters 366, 84-92 (2015).
30 Chen, K.-H. E. et al. Prolactin enhances T regulatory cell promotion of breast cancer through the long form prolactin receptor. Translational oncology 14, 101195 (2021).
31 White, C. A. et al. AID dysregulation in lupus-prone MRL/Fas lpr/lpr mice increases class switch DNA recombination and promotes interchromosomal c-Myc/IgH loci translocations: modulation by HoxC4. Autoimmunity 44, 585-598 (2011).
32 Rys, R. N., Venkataraman, M., Zeng, J., Mann, K. K. & Johnson, N. J. B. Fas Mutations in Non-Hodgkin's Lymphoma (NHL): Implications for Disease Progression and Therapeutic Resistance. Blood 134, 1520 (2019).
33 Gronbok, K. et al. Somatic Fas mutations in non-Hodgkin's lymphoma: association with extranodal disease and autoimmunity. Blood 92, 3018-3024 (1998).
34 Tsokos, G. C. Systemic Lupus Erythematosus. The New England Journal of Medicine 365, 2110-2121 (2011).
Malkiel, S., Barlev, A. N., Atisha-Fregoso, Y., Suurmond, J. & Diamond, B. Plasma cell differentiation pathways in systemic lupus erythematosus. Frontiers in immunology 9, 427 (2018).
36 Vincent, F. B., Morand, E. F., Schneider, P. & Mackay, F. The BAFF/APRIL system in SLE pathogenesis. Nature Reviews Rheumatology 10, 365-373 (2014).
37 Bekeredjian-Ding, I. B. et al. Plasmacytoid dendritic cells control TLR7 sensitivity of naive B cells via type I IFN. J Immunol 174, 4043-4050 (2005).
38 Cerutti, A., Qiao, X., He, B. J. I. & biology, c. Plasmacytoid dendritic cells and the regulation of immunoglobulin heavy chain class switching. Immunol Cell Biol 83, 554-562 (2005).
39 Ding, C., Cai, Y., Marroquin, J., Ildstad, S. T. & Yan, J. J. T. J. o. I. Plasmacytoid dendritic cells regulate autoreactive B cell activation via soluble factors and in a cell-to-cell contact manner. J Immunol 183, 7140-7149 (2009).
Huang, X., Dorta-Estremera, S., Yao, Y., Shen, N. & Cao, W. J. F. i. i. Predominant role of plasmacytoid dendritic cells in stimulating systemic autoimmunity. Front Immunol 6, 526 (2015).
41 William, J., Euler, C., Christensen, S. & Shlomchik, M. J. J. S. Evolution of autoantibody responses via somatic hypermutation outside of germinal centers. Science 297, 2066-2070 (2002).
42 William, J., Euler, C. & Shlomchik, M. J. J. T. J. o. I. Short-lived plasmablasts dominate the early spontaneous rheumatoid factor response: differentiation pathways, hypermutating cell types, and affinity maturation outside the germinal center. J Immunol 174, 6879-6887 (2005).
43 McDonnell, T. J. et al. Deregulated Bcl-2-immunoglobulin transgene expands a resting but responsive immunoglobulin M and D-expressing B-cell population. Molecular and Cellular Biology 10, 1901-1907 (1990).
44 Kipps, T. J. & Carson, D. A. Autoantibodies in chronic lymphocytic leukemia and related systemic autoimmune diseases. BLOOD-NEW YORK-81, 2475-2475 (1993).
Wardemann, H. et al. Predominant autoantibody production by early human B cell precursors. Science 301, 1374-1377 (2003).
46 Johnson, T. A., Rassenti, L. Z. & Kipps, T. J. J. T. J. o. I. Ig VH1 genes expressed in B cell chronic lymphocytic leukemia exhibit distinctive molecular features. J Immunol 158, 235-246 (1997).
47 Saini, S. S., Allore, B., Jacobs, R. M. & Kaushik, A. J. E. j. o. i. Exceptionally long CDR3H region with multiple cysteine residues in functional bovine IgM antibodies. Eur. J. Immunol 29, 2420-2426 (1999).
48 Tsai, A. G. et al. Human chromosomal translocations at CpG sites and a theoretical basis for their lineage and stage specificity. Cell 135, 1130-1142 (2008).
49 Muramatsu, M. et al. Class switch recombination and hypermutation require activation-induced cytidine deaminase (AID), a potential RNA editing enzyme. Cell 102, 553-563 (2000).
50 Mai, T. et al. Estrogen receptors bind to and activate the HOXC4/HoxC4 promoter to potentiate HoxC4-mediated activation-induced cytosine deaminase induction, immunoglobulin class switch DNA recombination, and somatic hypermutation. Journal of Biological Chemistry 285, 37797-37810 (2010).
51 Merino, R. n., Ding, L., Veis, D. J., Korsmeyer, S. J. & Nuñez, G. Developmental regulation of the Bcl-2 protein and susceptibility to cell death in B lymphocytes. The EMBO journal 13, 683-691 (1994).
52 Keenan, R. A. et al. Censoring of autoreactive B cell development by the pre-B cell receptor. Science 321, 696-699 (2008).
53 Osmond, D. G. Proliferation kinetics and the lifespan of B cells in central and peripheral lymphoid organs. Current opinion in immunology 3, 179-185 (1991).
54 Hummel, M. et al. A biologic definition of Burkitt's lymphoma from transcriptional and genomic profiling. New England Journal of Medicine 354, 2419-2430 (2006).
55 Lenz, G. et al. Stromal gene signatures in large-B-cell lymphomas. New England Journal of Medicine 359, 2313-2323 (2008).
56 Jais, J.-P. et al. The expression of 16 genes related to the cell of origin and immune response predicts survival in elderly patients with diffuse large B-cell lymphoma treated with CHOP and rituximab. Leukemia 22, 1917-1924 (2008).
57 Gu, Z. et al. PAX5-driven subtypes of B-progenitor acute lymphoblastic leukemia. Nature genetics 51, 296-307 (2019).
58 Rahman, M. A., Kramer, A. R. & Abdel-Wahab, O. SnapShot: splicing alterations in cancer. Cell 180, 208-208. e201 (2020).
59 Strati, P. & Shanafelt, T. D. Monoclonal B-cell lymphocytosis and early-stage chronic lymphocytic leukemia: diagnosis, natural history, and risk stratification. Blood, The Journal of the American Society of Hematology 126, 454-462 (2015).
60 Dang, C. V., Reddy, E. P., Shokat, K. M. & Soucek, L. Drugging the ‘undruggable’cancer targets. Nature Reviews Cancer 17, 502-508 (2017).
61 Walker, S. Treatment of systemic lupus erythematosus with bromocriptine. Lupus 10, 197-202 (2001).
62 Ben-Jonathan, N., Liby, K., McFarland, M. & Zinger, M. Prolactin as an autocrine/paracrine growth factor in human cancer. Trends in Endocrinology & Metabolism 13, 245-250 (2002).
63 Nagano, M. & Kelly, P. A. Tissue distribution and regulation of rat prolactin receptor gene expression. Quantitative analysis by polymerase chain reaction. Journal of Biological Chemistry 269, 13337-13345 (1994).
64 Swaminathan, S. et al. BACH2 mediates negative selection and p53-dependent tumor suppression at the pre-B cell receptor checkpoint. 19, 1014-1022 (2013).
65 Duy, C. et al. BCL6 is critical for the development of a diverse primary B cell repertoire. 207, 1209-1221 (2010).
66 Foster, M. H., MacDonald, M., Barrett, K. J. & Madaio, M. P. J. T. J. o. I. VH gene analysis of spontaneously activated B cells in adult MRL-lpr/lpr mice. J558 bias is not limited to classic lupus autoantibodies. 147, 1504-1511 (1991).
67 Anders, S., Reyes, A. & Huber, W. Detecting differential usage of exons from RNA-seq data. Nature Precedings, 1-1 (2012).
68 Love, M. I., Huber, W. & Anders, S. Moderated estimation of fold change and dispersion for RNA-seq data with DESeq2. Genome biology 15, 1-21 (2014).

### Example 2. LFPRLR as a Specific Therapeutic Target in Pathogenic B-Cell Subsets in SLE, and LFPRLR SMO could Specifically Eradicate Pathogenic Plasma Cells and their Precursor B Cells in SLE

### Introduction

SLE is a chronic autoimmune disease, with genetic, hormonal, and environmental factors contributing to disease manifestation. It is characterized by the overproduction and accumulation of abnormal, hyperactivated B cells and their terminally differentiated derivatives, plasma cells (Hiepe F, et al. Nat Rev Rheumatol. 2011 March; 7(3):170-178, and Malkiel S, et al., Front Immunol. 2018; 9:427). The autoantibodies produced by dysregulated plasma cells recognize a wide array of self-targets, including normal cellular DNA, RNA and proteins such as histones (Tsokos G C., et al. N Engl J Med. 2011 Dec. 1; 365(22):2110-2121), leading to tissue inflammation and malfunction. Current therapeutic approaches target plasma cells (the proteasome inhibitor, Bortezomib) or their B-cell precursors (pan B-cell depletion) but not both concurrently, and subject the patient to neurotoxicity or render them immunodeficient, respectively (Hiepe F, et al. Nat Rev Nephrol. 2016 April; 12(4):232-240, and Taddeo A, et al., Arthritis Res Ther. 2015 Mar. 2; 17(1):39). Described herein are less toxic approaches that concurrently target pathogenic plasma cells and their precursors in patients with SLE.

The extent to which PRL causally drives immune pathology in SLE is unknown. As shown in Example 1, among leukocyte compartments in SLE-prone mice, LFPRLR knockdown most markedly reduced the numbers of specific pathogenic B-cell subsets, including potentially autoreactive splenic CD19⁺ B cells with long immunoglobulin complementary determining regions, short-lived plasmablasts, and long-lived plasma cells (LLPCs) (Taghi Khani, et al. Commun Biol 6, 295 (2023)).

As described herein, our studies have (1) identified aberrant increases in the transcript expression of the LFPRLR in peripheral blood mononuclear cells (PBMCs) of patients with SLE and in SLE-prone mice, and (2) found that LFPRLR knockdown in SLE-prone mice reduces frequencies of germinal center-derived follicular (FO) B cells that give rise to autoimmune class-switched plasma cells (Dema B, et al., Antibodies. 2016 Jan. 4; 5(1):2) while concomitantly increasing percentages of marginal zone (MZ) B cells. 5′ and 3′ single cell RNA sequencing of splenocytes from SLE-prone mice with and without LFPRLR knockdown, have identified plasma cells as the pathogenic splenic immune subset that expresses the highest levels of PRLR, with LFPRLR as the predominant PRLR isoform. As described herein, studies of Example 1 and the instant Example suggest that the level of expression of the LFPRLR could differentiate between normal and autoimmune B-cell subsets. Based on the above, knocking down expression of the LFPRLR would be an advance, over current therapies, by simultaneously and specifically decreasing both autoimmune B cells and their derivative plasma cells.

Current treatments for SLE include anti-inflammatory and immunosuppressive medications and pan B-cell depleting regimens that alleviate symptoms and slow down tissue damage. However, sustained remission of SLE remains a clinical challenge for several reasons. First, B-cell depleting regimens do not eradicate the existing autoantibody-producing, short-lived plasmablasts and long-lived plasma cells (LLPCs), both of which drive SLE pathology. Second, proteosome inhibitors such as bortezomib eradicate plasma cells but not their less mature pathologic B-cell precursors (Hiepe F, et al. Nat Rev Nephrol. 2016 April; 12(4):232-240) in germinal centers (Brynjolfsson S F, at al. Front Immunol. 2018 Nov. 16; 9:2673). Third, these agents and anti-inflammatory medications (steroids) kill both pathologic and normal immune cells in patients, creating problems with generalized immunosuppression. In contrast, LFPRLR SMO may specifically and concurrently eradicate abnormal B-cell precursors that give rise to new pathogenic plasma cells and existing pathogenic plasma cells in SLE without effect on normal cells.

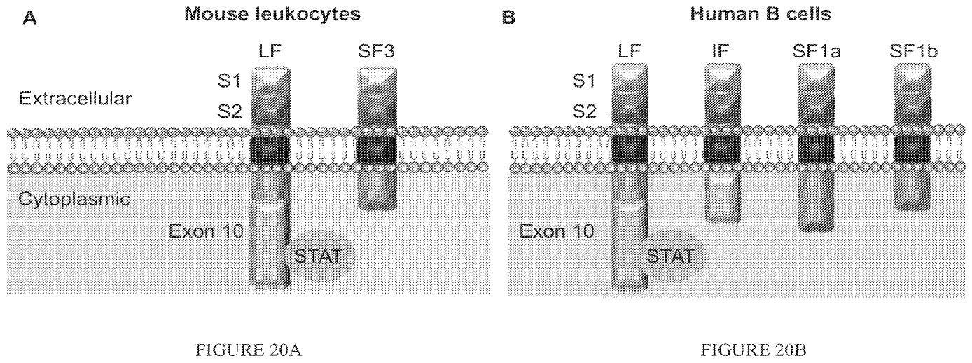

Two categories of PRLRs are formed by alternative splicing of a single PRLR pre-mRNA in mice and humans, generating the long (LF) and short (SF) PRLR isoforms (Trott J F, et al., J Mammary Gland Biol Neoplasia. 2008 March; 13(1):3-11 and Bouilly J, et al., Mol Cell Endocrinol. 2012 Jun. 5; 356(1-2):80-87). In humans, there is an intermediate form (IF), also produced by alternative splicing (Grible J M, et al., NPJ Breast Cancer. 2021 Mar. 26; 7(1):37), at least in pathological specimens. LF and SF PRLRs in both species have identical extracellular and transmembrane domains. They only differ in the intracellular signaling domains: LF and IFPRLR include the entire or a portion of exon 10, respectively, which is missing in the SFPRLRs. As a result, the LFPRLR can activate STATs, whereas the SFPRLRs cannot (Abramicheva P A, et al. Biochem Biokhimiia. 2019 April; 84(4):329-345) (see FIG. 20).

We knocked down the LFPRLR in MRL-lpr mice, which develop symptoms of systemic autoimmunity at 13 weeks and succumb to SLE at 17 weeks of age. LFPRLR knockdown reduced the number of splenic B cells, dendritic cells (DCs), plasmablasts, and LLPCs, and reduced circulating anti-double stranded (ds) DNA autoantibodies (see Examples 1 and 4). Among splenic B-cell subsets, knockdown of LFPRLR specifically reduced (see Example 1) the potentially autoreactive B cells with long complementary determining regions 3 (CDR3s) of >20 amino acids (Wardemann H, et al. Science. 2003; 301(5638):1374-1377). LFPRLR knockdown also suppressed the expression of AID (activation induced cytidine deaminase) (see Example 1), an enzyme which is important for the production of pathogenic, class-switched plasma cells in SLE. Therefore, the signaling of PRL through the LFPRLR on B cells in SLE may (1) promote germinal center reactions leading to production of newly class-switched autoantibody-secreting plasma cells, and (2) may be anti-apoptotic, resulting in the accumulation of autoantibody-producing plasma cells.

Thus, LFPRLR SMO has the unique ability to specifically reduce B cells with long CDR3s that are potentially autoimmune, reduce numbers of existing plasma cells (see Example 1), and concurrently suppress the germinal center reaction that produces new plasma cells. Thus, LFPRLR SMO has advantages over pan B-cell depleting therapies, global immunosuppressants such as glucocorticoids, and toxic proteosome inhibitors currently used to alleviate SLE in patients.

### The LFPRLR is Aberrantly Overproduced in Mouse and Human SLE

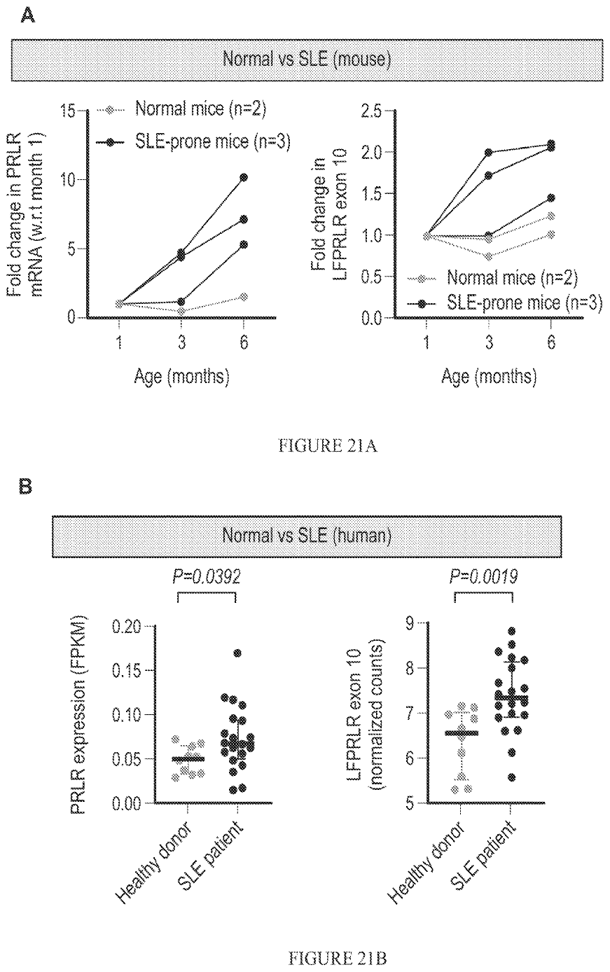

Whether increased expression of PRLRs on leukocytes could contribute to enhanced PRL signaling and thereby, be associated with SLE pathogenesis had not been determined. Therefore, we measured how transcript expression of total PRLR, and its isoforms changes with disease progression in splenic leukocytes of SLE-prone mice and upon development of SLE in human patients. We found aberrant increases in the expression of total PRLRs during disease progression in splenic leukocytes of female NZB/W mice that become moribund with symptoms of SLE (autoantibodies, glomerulonephritis, proteinuria) at ˜8 months of age (FIG. 21A, left), and in peripheral blood mononuclear cells (PBMCs) of newly diagnosed SLE patients [19 females and 1 male, (FIG. 21B, left)]. Using bioinformatic methods (Anders S, et al. Genome Res. 2012 October; 22(10):2008-2017 and Love M I, et al. Genome Biol. 2014; 15(12):550) to parse the relative expression of the PRLR isoforms from RNA sequencing data as we did in Example 1 (also see Taghi Khani, et al. Commun Biol 6, 295 (2023)), we found that murine and human leukocytes in both normal and SLE conditions expressed predominantly the LFPRLR [expression of SFPRLR was below the detection sensitivity of RNA sequencing (FIGS. 21A-21B, right)]. Thus, we infer that LFPRLR is aberrantly overproduced in murine and human SLE.

### Knockdown of LFPRLR Affects the Process of Plasma Cell Generation in SLE-Prone Mice

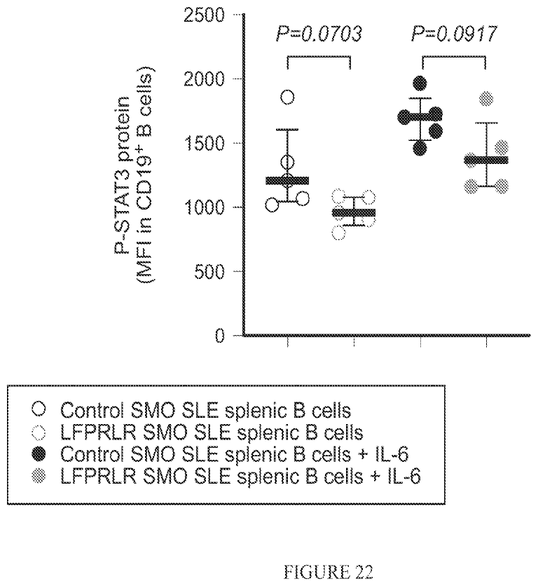

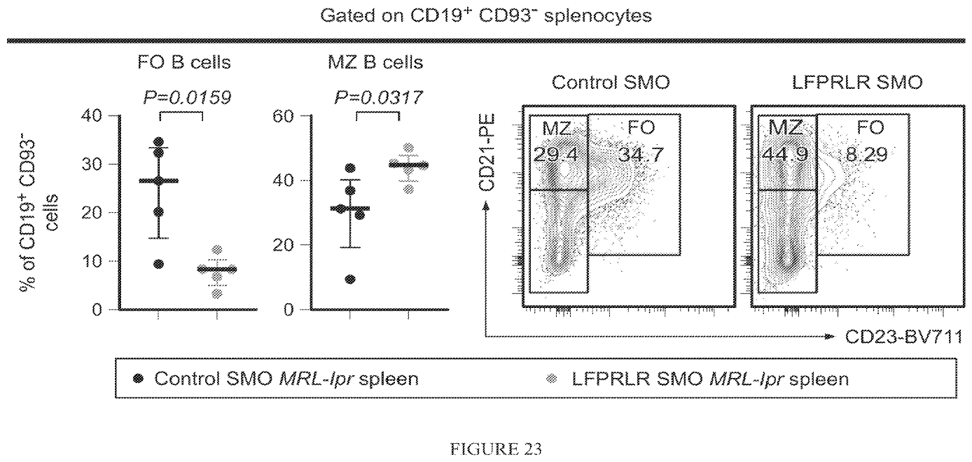

Plasma cells are a terminally differentiated subset of B cells that produce and secrete antibodies. These cells arise from B-cell predecessors called ‘mature’ B cells in secondary lymphoid tissues, including the spleen. Mature B cells, in response to foreign antigens (during infections) or self-antigens (in SLE) trigger phosphorylation and activation of STAT3, which in turn promotes B-cell proliferation, somatic hypermutation (SHM) and class switch recombination (CSR), and subsequent differentiation into plasma cells in splenic germinal centers (GCs). This process of plasma cell production is further aided by other immune cells in GCs, including DCs that produce IL-6 (a stimulator of PRL) (Spangelo B L, et al. Endocrinology. 1989 July; 125(1):575-577). The extent to which PRL and its STAT-binding, pro-proliferative, anti-apoptotic LFPRLR isoform modulate the process of plasma cell generation in SLE is unknown. By knocking down LFPRLR in MRL-lpr SLE-prone mice (Taghi Khani, et al. Commun Biol 6, 295 (2023)), we found that activation of STAT3 in splenic B cells could at least partly depend on the expression of the LFPRLR on these cells (FIG. 22). We then measured whether knockdown of LFPRLR in SLE-prone mice differentially impacts the frequencies of the two mature B-subsets, follicular (FO) and marginal zone (MZ) B cells, which give rise to potentially highly autoreactive, class-switched plasma cells and potentially normal, non-class-switched plasma cells, respectively. We found reduced representation of splenic FO B cells and a concomitant rise in the representation of MZ B cells after LFPRLR knockdown in SLE-prone mice (FIG. 23). The above findings are concordant with Example 1 studies (also see Taghi Khani, et al. Commun Biol 6, 295 (2023)) showing that knockdown of LFPRLR in SLE-prone mice resulted in a reduction in number and proliferation of mature splenic B cells, which are precursors of plasma cells, reduced numbers of DC subsets, which help in B-cell proliferation, reduced expression of the AID enzyme that facilitates SHM and CSR, and reduced numbers of antibody-secreting plasma cell subsets.

### Plasma Cells: a Leukocyte Subset Highly Vulnerable to LFPRLR Knockdown

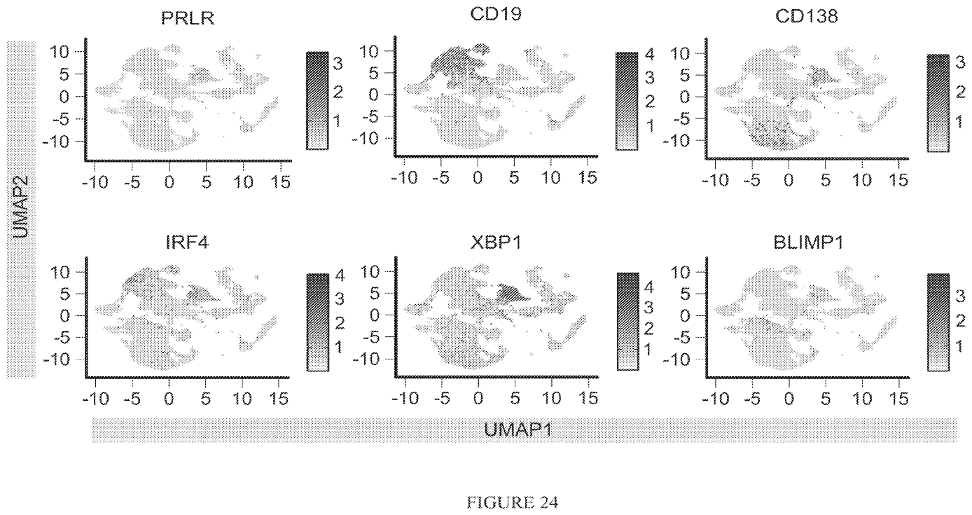

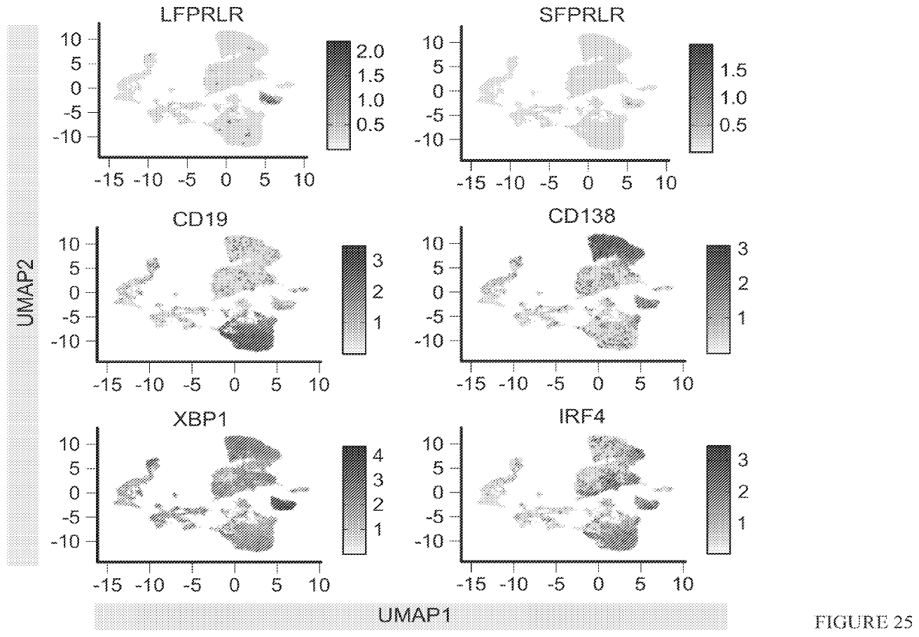

In the human circulation, B cells express LF/IF and SFPRLRs, whereas effector T and NK cells do not (Taghi Khani, et al. Commun Biol 6, 295 (2023)). Concordant with previous findings in Example 1, 5′-single cell RNA sequencing (scRNA-seq) of splenocytes from SLE-prone mice identified that total PRLR is most highly expressed in B-cell subsets. Surprisingly, within the B-cell population in SLE-prone mice, CD138⁺ XBP1⁺ IRF4⁺ BLIMP1⁺ plasma cells, that can produce autoantibodies, were by far the major expressors of PRLRs (FIG. 24). Using 3′-scRNA-seq, we then quantified transcripts of LF and SF PRLR (which include or do not include the 3′ exon 10, respectively), in splenic leukocytes of SLE-prone mice. Consistent with our earlier findings (Example 1 and FIG. 24), plasma cells of SLE-prone mice express the most PRLR, and of the isoforms, the LFPRLR is much more abundantly expressed than the SFPRLR (FIG. 25). Based on this expression pattern, plasma cells in SLE will be particularly vulnerable to LFPRLR knockdown.

### LFPRLR Knockdown is not Toxic to Normal Human Plasma Cells

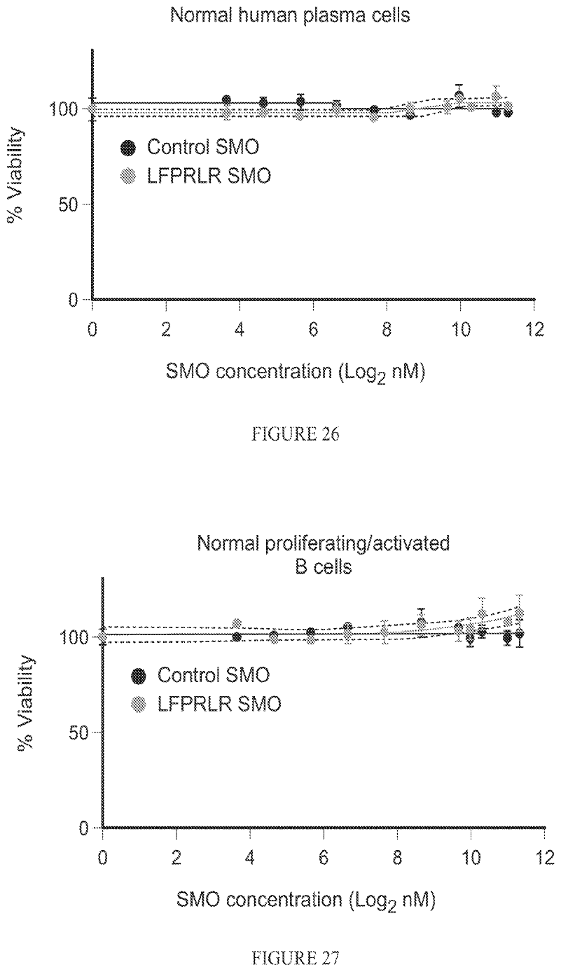

The viability of normal plasma cells differentiated from PBMCs of healthy donors was unaffected by LFPRLR knockdown (FIG. 26). Thus, LFPRLR SMO may be an attractive approach to specifically eradicate autoimmune plasma cells. Both normal proliferating/activated B cells and their plasma cell derivatives from healthy donor PBMCs are not sensitive to LFPRLR knockdown (see FIG. 26 for normal plasma cell-subsets and FIG. 27 for normal proliferating/activated B cells).

### Example 3. LFPRLR Knockdown May be Used as a Targeted Single Agent Approach for Patients with SLE that May Concurrently Alleviate B- and Plasma-Cell Pathology and Sustain Disease Remission

### Determine the Effect of LFPRLR Knockdown on Pathogenic B-Cell Subsets in Murine SLE.

We will measure the effects of LFPRLR knockdown on phenotype, production, apoptosis, and/or cycling of plasma cells in the NZB/W SLE-prone mouse model. NZB/W mice experiments will provide additional, complementary pre-clinical information. Fas-mediated apoptosis is intact in NZB/W mice, allowing us to further assess the effects of LFPRLR knockdown on the apoptosis of pathogenic B-cell subsets. Female NZB/W mice succumb to SLE at ˜34 weeks of age, which is 6 months earlier than their male counterparts. Taking into consideration the time differences between males and females to succumb to SLE, we will treat 20-week-old female and 44-week-old male NZB/W mice with either a control SMO (a morpholino oligonucleotide with octaguanidine derivatization but with no target) or LFPRLR SMO via 4-week Alzet minipumps (see Example 1). A total of n=36 mice per group, 9 males and 27 females will be used.

The pump, delivering control SMO or LFPRLR SMO, will be replaced when female and male mice are 24 and 48 weeks old, respectively. To study the effects of LFPRLR knockdown on plasma cell pathology, NZB/W mice should not succumb to SLE. Hence, we will euthanize female and male mice at 28 and 52 weeks of age, respectively, and harvest splenic leukocytes for profiling plasma cell homeostasis by scRNA-seq and high dimensional flow cytometry. All animals will be administered 5-ethynyl-2′-deoxyuridine (EdU) two hours before sacrifice to allow for analysis of proliferation of cells by flow cytometry.

We will conduct 5′- and 3′-scRNA-seq on six splenic samples (3 females, 3 males) each from control SMO- and LFPRLR SMO-treated mice. Both 5′- and 3′-scRNA-seq will allow us to compare, in SLE-prone mice with and without LFPRLR knockdown the: (1) frequencies, numbers, and phenotype (Chen H, et al. Cell Mol Immunol. 2019 March; 16(3):242-249) of plasma cells and their transitional, FO, and MZ B-cell precursors, and (2) cell cycle status and indices of apoptosis (Hsiao C J, et al. Genome Res. 2020 April; 30(4):611-621) of B-cell subsets. As in FIGS. 24-25, plasma cells will be identified using markers such as CD138, Blimp1/PRDM1, IRF4, and XBP1 (expressed on/inside plasma cells), CD19, CD20, CD22, and B220 (expressed on the less mature B-cell predecessors of plasma cells). We will also compare the frequencies of IgG/IgA/IgE-secreting class-switched plasma cells and IgM/IgD-secreting non-class-switched ones. 5′-scRNA-seq will also allow us to measure the effects of LFPRLR knockdown on the length and amino acid (Huang W, et al., JCI Insight. 2018 Sep. 6; 3(17):122525) distributions of Ig heavy chain CDR3. Changes in the expression of PRLR isoforms in B-cell subsets before and after LFPRLR knockdown can be determined by 3′-scRNA-seq as in FIG. 25.

We will validate the transcriptomic changes observed in scRNA-seq experiments using flow cytometry to measure intracellular and surface proteins in splenic B-cell subsets in instances where antibodies are available. Flow cytometry will be conducted in at least 23 female and 6 male mice in control SMO- and LFPRLR SMO-treated groups.

As seen in FIGS. 24-25, plasma cells in this Example are expected to express the highest levels of PRLRs and predominantly the LFPRLR. We expect the reduction in plasmablasts and LLPCs after LFPRLR knockdown as seen in MRL-lpr SLE-prone mice (see Example 1) to be enhanced in NZB/W mice as the latter have intact Fas-mediated apoptosis. Our studies will determine whether such reduction in plasma cell numbers after LFPRLR SMO treatment arises from increased apoptosis and/or reduced proliferation of plasma cells and/or altered differentiation of B cells into plasma cells. In NZB/W mice subjected to LFPRLR knockdown, we anticipate a reduction in the expression of AID (see Example 1) and in the frequencies of FO B cells (FIG. 23), as seen in MRL-lpr mice. A reduction in AID and FO B cells and an increase in MZ B cells after LFPRLR knockdown would suggest a reduction in the production of class-switched plasma cells through germinal center reactions. Comparisons of frequencies of plasma cells with class-switched and non-class-switched transcriptional signatures (Price M J, et al., J Immunol. 2019 Oct. 15; 203(8):2121-2129) in our 5′-scRNA-seq data will yield further insights into the extent to which LFPRLR knockdown reduces the production of potentially pathogenic class-switched plasma cells (Dema B, et al., Antibodies. 2016 Jan. 4; 5(1):2). Comparisons of lengths and amino acid compositions of IgH CDR3 and of isotypes of constant regions expressed in mature B cells will provide similar insights: we predict that knockdown of LFPRLR will reduce frequencies of B cells with CDR3>20aa and charged amino acids, indicative of autoreactivity, as well as reduce the frequencies of class-switched (IgG/E/A⁺) B cells.

Additionally, we will determine the preclinical efficacy of LFPRLR SMO in directly reducing plasma cell numbers and alleviating their pathologic/autoimmune consequences such as increased circulating anti-dsDNA antibodies, proteinuria, and glomerulonephritis. To this end, we will transplant 8-week-old female Rag1−/− immune-deficient mice with antibody-producing splenic plasma cells from 28-week-old female NZB/W SLE-prone mice, as shown previously (Cheng Q, et al. Ann Rheum Dis. 2013 December; 72(12):2011-2017). NZB/W plasma cells have been shown to proliferate within 14 days of transplantation into Rag1−/− transplant recipient mice. Hence, we will treat 10-week-old plasma cell transplant recipients with control SMO or LFPRLR SMO for 8 weeks. Because female NZB/W mice develop SLE symptoms such as autoantibody-producing splenic plasma cells 6 months earlier than their male counterparts, they will be used as transplant donors. Changes in frequencies of circulating plasma cell subsets and anti-dsDNA antibodies will be measured monthly after transplantation using previously reported methods (Cheng Q, et al. Ann Rheum Dis. 2013 December; 72(12):2011-2017) and/or methods described herein (e.g., see Example 4). In euthanized mice from both groups, we will compare the extent of glomerulonephritis by immunohistochemistry, proteinuria by dipstick urinalysis, and plasma cell numbers and cycling in the spleen, peripheral blood, bone marrow, and/or kidney by flow cytometry.

Examples 1-2 data suggests that some indicators of SLE progression may be altered by treatment with LFPRLR SMO. That is, in mice transplanted with plasma cells from SLE-prone NZB/NZW mice, we expect LFPRLR knockdown to reduce indicators of SLE progression that include circulating plasma cells and anti-dsDNA antibodies. We also expect treatment with LFPRLR SMO to reduce glomerulonephritis, proteinuria, and numbers of splenic, bone marrow, circulating, and/or kidney plasma cells.

Our qPCR results show that CD19+ B cells do express very low levels of LF and SFPRLR (with a ratio of LF:SF=9:1) (Taghi Khani, et al. Commun Biol 6, 295 (2023)). However, in 5′- and 3′-scRNA-seq experiments, total PRLR and its isoforms were only detected on CD19⁻ plasma cells (FIGS. 24-25). Because we found that minute levels of LFPRLR (not detected in scRNAseq) in less mature CD19⁺ B cells significantly impact SLE B-cell pathology (Taghi Khani, et al. Commun Biol 6, 295 (2023)), we will supplement scRNA findings by using qPCR to compare mRNA levels of PRLR isoforms in sorted CD19⁺ B cells and the more mature CD19⁻ plasma cells before and after LFPRLR knockdown.

### Determine the Ability of LFPRLR Knockdown to Reduce Pathogenic B-Cell Subsets in Human SLE.

We will compare the secretion of local PRL and the expression of local PRL and PRLR isoforms (LF, IF, SF1a, SF1b) by qPCR in sorted existing B cells, in expanded activated B cells, and in plasma cell subsets differentiated from PBMC B cells (Cocco M, et al., J Immunol. 2012 Dec. 15; 189(12):5773-5785) of 20 healthy donors and of 30 patients with SLE. Patient samples will be obtained from individuals who are >11-years-old (˜n=10 adolescents and young adults and ˜n=20 older adults), to limit age from being such a large variable while comparing healthy donors (who will be mostly adults) and patients with SLE. To account for the female skewing of SLE, we will compare ˜15 females/5 male healthy donors with ˜24 females/6 male patients with SLE.

SLE samples will include those collected from newly diagnosed treatment-naïve patients and, because there will not be many of those, patients who have been on very short-term steroid treatment for up to a week after diagnosis (˜n=10); patients who have been on steroid treatments for 1-2 months (˜n=10); as well as patients with active SLE who have not benefitted from long-term steroid treatments for >2 months and are therefore, potential candidates for receiving B-cell depleting therapies (˜n=10). Samples from patients on B-cell depleting therapies will not be used, as measurements of B-cell subsets would likely not be possible.

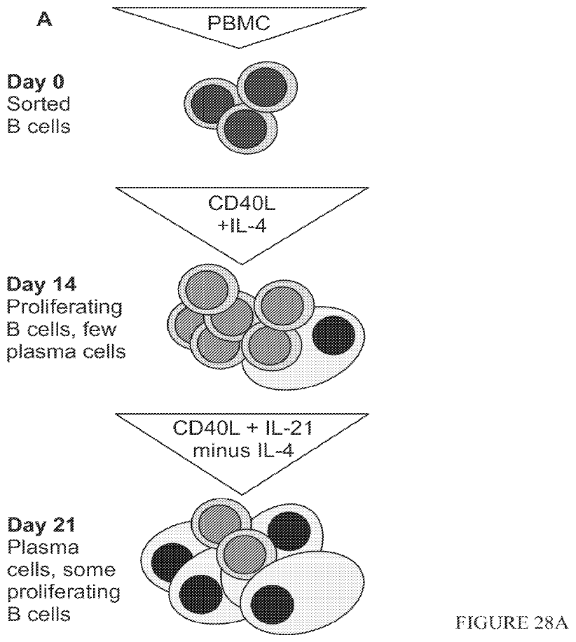

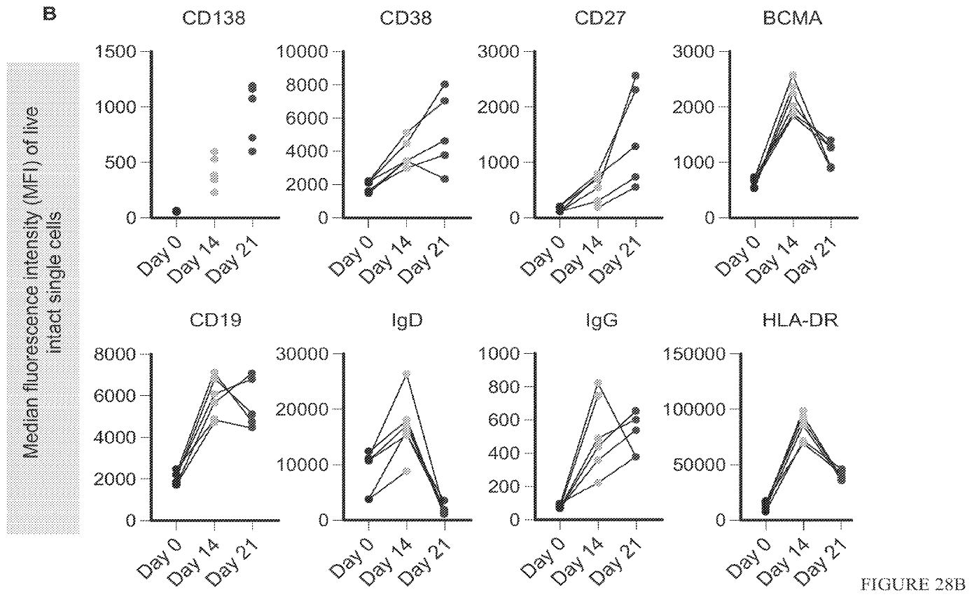

Human B-cell expansion and activation from PBMCs, in vitro differentiation of PBMC B cells into plasmablasts and LLPCs (Cocco M, et al. J Immunol 2012 Dec. 15; 189(12):5773-5785) and the validation of successful plasma cell generation (Khoenkhoen S, et al. Front Immunol. 2020; 11:571321) will be carried out using our standardized protocol described below (see FIG. 28). Human multiple myeloma cell lines were used as positive controls to validate successful human LLPC production by flow cytometry of cell surface markers (data not shown). As shown in Examples 1 and 4 study on B-cell lymphomas, we will use ELISA to compare the levels of autocrine/paracrine (local) PRL secreted from PBMCs of healthy donors and SLE patients.

We will collect and viably freeze multiple aliquots of PBMC samples from 30 patients with SLE. To determine vulnerability of each PBMC sample to LFPRLR knockdown, we will measure the ability of LFPRLR SMO to reduce viable cell numbers using MTT assays (see FIGS. 26, 27 for examples). Samples will be treated for 48 h with concentrations ranging from 0 nM to 5 mM control SMO or LFPRLR SMO as used in studies described herein. MTT assays will be used to determine the half-maximal inhibitory concentration (IC50) of the LFPRLR SMO for each sample.

Next, we will determine which immune fraction(s), present in PBMCs of patients with SLE, are the most vulnerable to LFPRLR knockdown by using flow cytometry to measure the viability of specific gated immune compartments. Immune subsets measured will include B-cell subsets (the less mature CD19⁺ B-cell predecessors and their terminally differentiated CD19⁻ CD138⁺ CD3⁻ CD56⁻ plasma cell derivatives), CD3⁺ total T cells, and CD3⁻ CD56⁺ total NK cells. Like that shown for healthy B-cell subsets in FIGS. 26,27, using flow cytometry, we will also determine the extent to which expanded B cells (day 14 FIG. 28) and plasma cell subsets (day 21 FIG. 28) differentiated from SLE PBMC B cells are eradicated by treatment with LFPRLR SMO. Concentrations of control- and LFPRLR-SMOs in flow cytometry assays will be the IC50 of LFPRLR SMO in each patient sample as determined by the MTT assay. For PBMC samples found not to secrete local (autocrine and paracrine) PRL by ELISA, MTT and flow cytometry assays will also be conducted in the presence of added 200 ng/ml exogeneous human PRL. The addition of exogenous PRL will mimic increased levels of circulating (endocrine) or stromal paracrine PRL that may have been present in the patient.

Based on FIGS. 21B, 24, and 25, B-cell subsets present in, and those differentiated from, PBMCs of patients with SLE will have increased expression of total PRLR and the LF/IFPRLR compared to their normal counterparts in healthy donors. Whole RNA sequencing of PBMCs was not sensitive enough to detect the expression of the SFPRLRs (FIG. 21). However, because normal human B cells express very low levels of counteractive SFPRLRs that are detectable by qPCR (Taghi Khani, et al. Commun Biol 6, 295 (2023)), our qPCR studies may find expression of SFPRLR to be either unchanged or reduced in B-cell fractions of SLE patient PBMCs. A possible outcome is that PBMC B-cell subsets of SLE patients express only LF/IFPRLR and no counteractive SFPRLRs, unlike normal B cells that we know express LF and SFs (Taghi Khani, et al. Commun Biol 6, 295 (2023)). Nevertheless, all the above outcomes will point to an increased and specific vulnerability of B-cell subsets within the SLE PBMCs to LFPRLR knockdown.

PBMCs from patients with SLE may or may not transcribe and secrete local (autocrine and paracrine) PRL. If we observe no changes in the expression of local PRL and relative PRLR isoforms between normal and SLE samples, we will infer that increases in endocrine PRL and in total and LF/IF PRLR (e.g., FIG. 21B) are sufficient to elicit aberrant immune hyperactivation in patients with SLE.

Unlike total PBMCs from healthy donors, which we show are insensitive LFPRLR knockdown (Taghi Khani, et al. Commun Biol 6, 295 (2023)), we expect PBMCs from patients with SLE to be vulnerable to LFPRLR knockdown because the population as a whole aberrantly overexpresses PRLRs (FIG. 21B). Subsequent flow cytometry experiments will further assess whether reduced fitness of human SLE PBMC samples after LFPRLR knockdown is caused by a reduction in all or specific pathogenic immune subsets. Examples 1 and 4 (Taghi Khani, et al. Commun Biol 6, 295 (2023)) and Example 2 in mice prone to SLE find LFPRLR knockdown to specifically reduce pathogenic B-cell subsets and PRLR expression to be the highest in plasma cells (FIGS. 24-25). Based on this, we expect treatment with LFPRLR SMO to selectively (or more markedly) kill the pathogenic B-cell subsets within the PBMC population of human patients with SLE.

Differentiation Protocol of Plasma Cells from Human PBMCs

### Day 0:

B cell Isolation and culture in Stem MACS HSC Expansion Media XF (+ FBS+ IL-4+ CD40-ligand)
Flow cytometry measurement of B-cell markers
Day 0: Culture 0.15×10⁶ B cells in 24 well containing 1 ml complete media.

### Day 14:

Harvest B cells for flow, MTT assays, and qPCR for PRLR isoforms (these will be a mixture of naïve, activated B cells, and some plasma cells).
For remaining cells, wash and replace with fresh media (switch to IL-21 instead of IL-4 to enhance plasma cell proportion)

### Day 21:

Harvest B cells for flow, MTT assays, and qPCR for PRLR isoforms (these will be a mixture of naïve, activated B cells, and plasma cells. In comparison to day 14, more cells are switching to plasma cell phenotype)
   The above protocol uses the B-cell expansion kit from Miltenyi on days 0-14 but mimics the Modified B cell expansion kit from Days 14-21
Scale up day 0 depending on starting number of cells
Cells are restimulated on Days 5, 7, 10, 14, 17 or 18

### Example 4. Isoform-Specific Knockdown of Long and Intermediate Prolactin Receptors Interferes with Evolution of B-Cell in Subject with B Cell Disorder(s)

With the experimental setups as described above in Example 1, additional data are described herein.

Anti-dsDNA Autoantibody Production Trended Towards a Reduction after LFPRLR Knockdown.

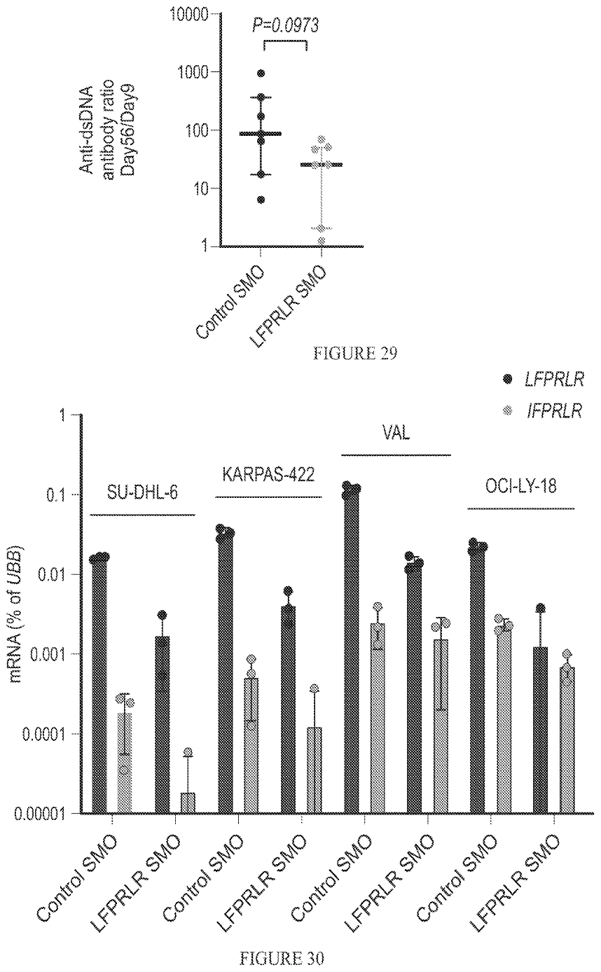

Whether PRL promotes anti-dsDNA autoantibody production by signaling specifically through the LFPRLR was not determined. Hence, we determined whether LFPRLR knockdown impacted the production of anti-dsDNA autoantibodies in serum of SLE-prone mice. Although the early stage of disease analyzed makes the Mrl-lpr model less than ideal for this measurement, levels of anti-dsDNA autoantibodies trended towards reduction after LFPRLR knockdown (FIG. 29), which matches with the reduction in plasma cell subsets seen earlier. Our findings lead us to conclude that LFPRLR knockdown reduces numbers of pathologic splenic B-cell subsets in SLE-prone mice.

### STAT Signaling

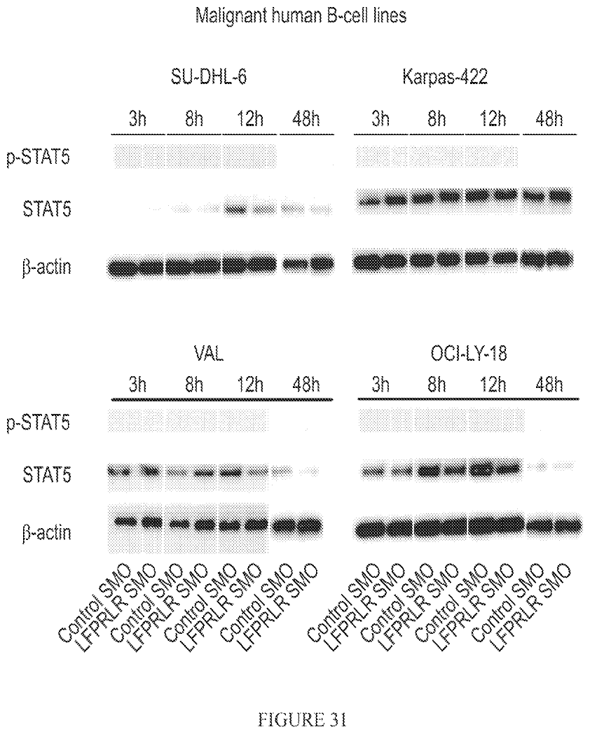

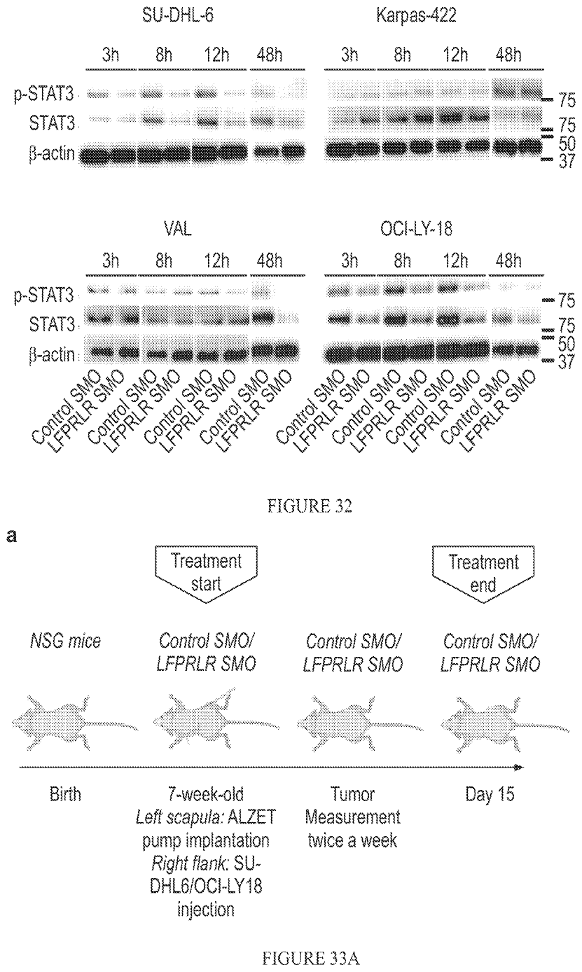

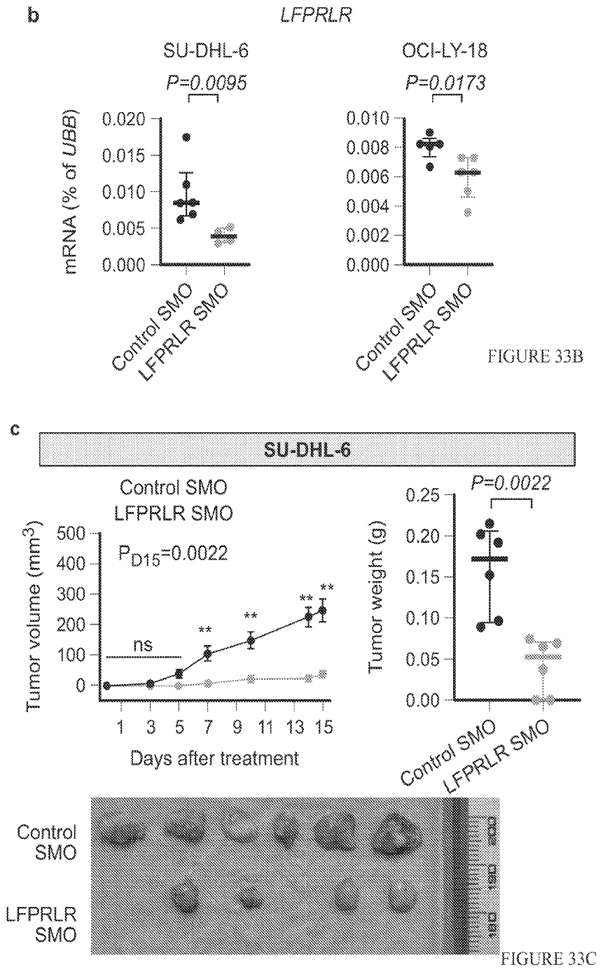

We investigated the mechanisms by which LFPRLR knockdown reduces the fitness of malignant B cells. To determine whether suppression of STAT signaling is a potential mechanism of action of the LFPRLR SMO, we compared expression of the ‘STAT-activating’ LFPRLR and the ‘non-STAT-activating’ IF PRLR (Grible, J. M. et al. NPJ Breast Cancer 7, 1-11 (2021)) relative to one another and their absolute expressions before and after LFPRLR knockdown. Expression of both LF and IF PRLR decreased after treatment with LFPRLR SMO. However, LFPRLR expression was ˜10-80-fold higher than IF expression in each cell line in the absence of treatment, suggesting that LFPRLR SMO may kill malignant lymphocytes by reducing the opportunity for STAT activation by PRL (FIG. 30). Phosphorylation (activation) and production of STAT3 decreased after LFPRLR knockdown in three out of four malignant B cell lines treated with their cell death inducing IC50 concentrations of LFPRLR SMO (FIG. 32). pSTAT5 was undetectable in all cell lines, although levels of global STAT5 were reduced in some lines after treatment with LFPRLR SMO (FIG. 31). Our results are consistent with the recent finding that STAT3, and not STAT5, is activated downstream of PRLR in autoimmune B cells (Flores-Fernández, R. et al. Cells 10, 316 (2021)). We conclude that activation of STAT3 downstream from LFPRLR may play an important role in sustaining B-cell malignancies.

### Knockdown of LFPRLR in DLBCL-Prone Mice Suppresses TCL1 Oncoprotein

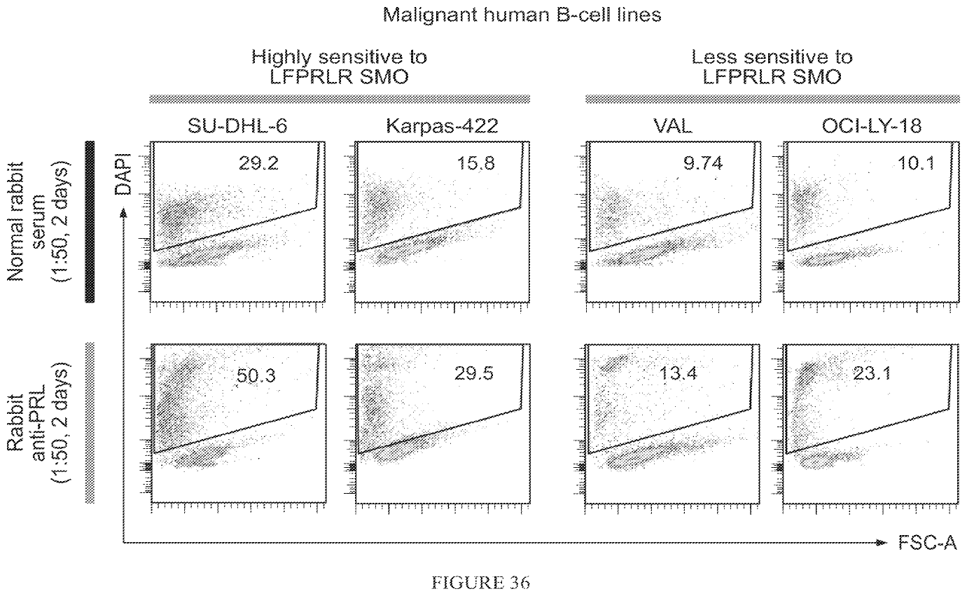

LFPRLR knockdown significantly reduced expression of the driver TCL1 oncoprotein in splenic B cells in the DLBCL-prone mice (FIG. 35). Hence, abrogation of LFPRLR synthesis in 16-week-old TCL1-tg-mice specifically impacts pre-malignant B cells and reduces the major indicators of B-cell lymphoma risk specific to this age group.

### Knockdown of LFPRLR Reduces Progression of Overt Human DLBCL In Vivo

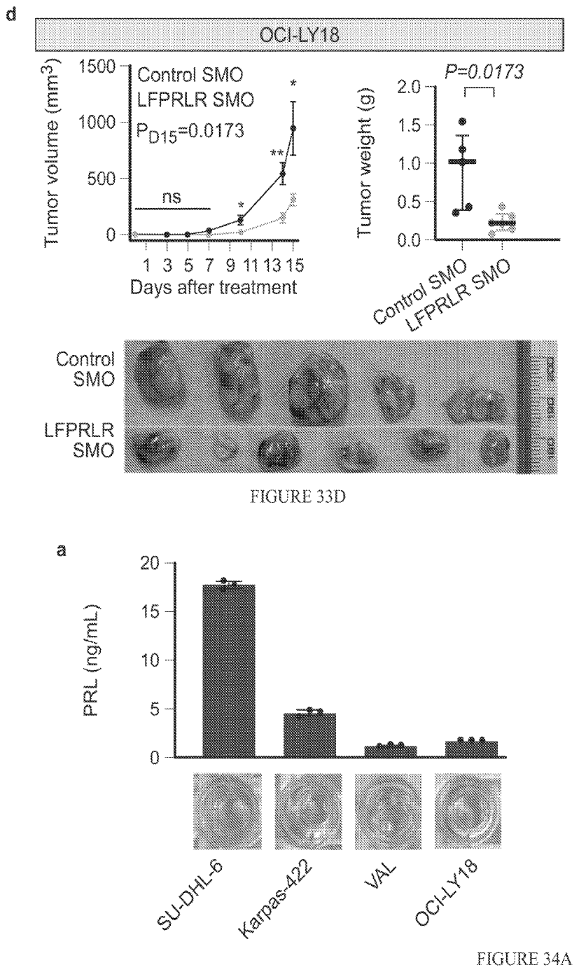

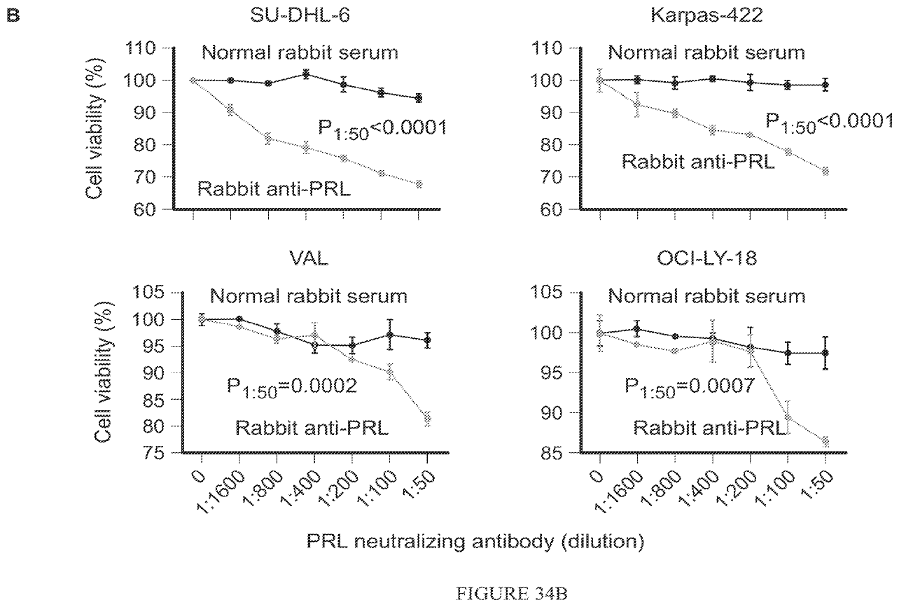

To further demonstrate that knockdown of LFPRLR could be therapeutically beneficial by inhibiting progression of overt B-cell malignancies, we tested the efficacy of LFPRLR SMO in the clearance of two human DLBCL cell-line derived xenografts (CDX) transplanted into immune-deficient NOD-SCID IL2Rγ−/− (NSG) mice. Control SMO or LFPRLR SMO were continuously delivered via alzet minipump for 15 days (FIG. 33a). We confirmed that LFPRLR was significantly knocked down in tumors isolated from mice treated with LFPRLR SMO (FIG. 33b). Consistent with our short-term in vitro studies (FIG. 5i), we observed that LFPRLR knockdown significantly slowed DLBCL progression in vivo in the two independent CDX models used (FIG. 33c, d). We conclude that the knockdown of LFPRLR effectively reduces the progression of overt B-cell malignancies. Because the human LFPRLR SMO does not affect mouse LFPRLR (Utama, F. E. et al. Endocrinology 150, 1782-1790 (2009)) we also conclude that this result stems from a direct effect on the B-cell lymphomas. At the same dose of transplanted lymphoma cells, the tumors derived from SU-DHL-6 were more sensitive to LFPRLR knockdown than those derived from OCI-LY18, consistent with the in vitro results (FIGS. 5i and 33c, d).

### Knockdown of LFPRLR in Malignant Human B Cells Reduces BCL2 and MYC Expression

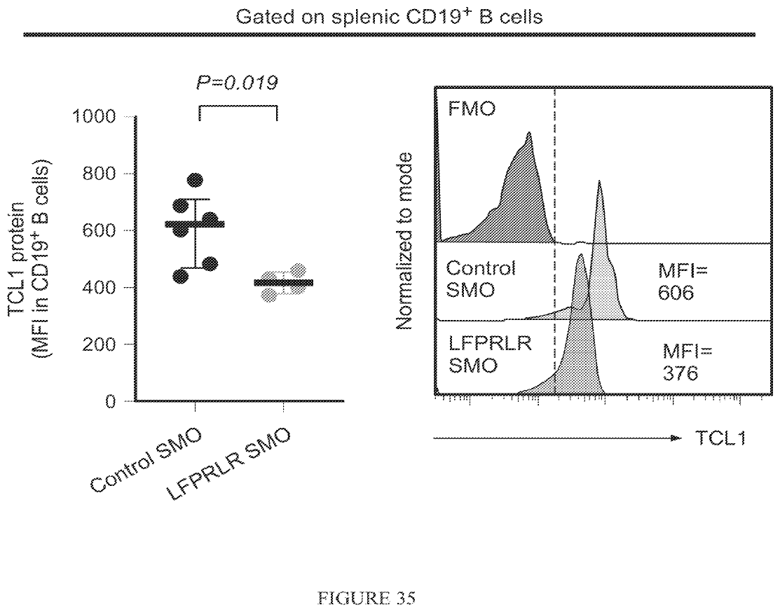

Given the differential sensitivity of the cell lines to LF/IFPRLR knockdown both in vitro and in vivo, the intrinsic properties of malignant B cells, including their level of autocrine PRL secretion and expression of oncogenic drivers may dictate their sensitivity to increasing concentrations of LFPRLR SMO. First, we determined whether differential secretion of PRL from malignant B cells was related to their sensitivity to LFPRLR knockdown. Cells sensitive to nM concentrations of LFPRLR SMO (SU-DHL-6 and Karpas-422, FIG. 5i) secreted much higher levels of PRL than their counterparts sensitive only to μM concentrations (VAL and OCI-LY-18, FIG. 5i) (FIG. 34a). Importantly, neutralization of the secreted PRL with rabbit anti-PRL, even at low concentrations, markedly reduced the viability of cell lines with high amounts of PRL secretion and nM sensitivity to LFPRLR SMO (FIG. 34b, FIG. 36). In contrast, in malignant B cells sensitive to μM concentrations of LFPRLR SMO, significant impairment of cellular fitness was only observed at high concentrations of anti-PRL (FIG. 34b, FIG. 36). Hence, malignant B cells' dependence on LFPRLR positively correlates with their secretion of autocrine PRL.

### Methods

### Enzyme Linked Immunosorbent Assays (ELISA)

For the quantitative determination of human PRL concentrations in cell supernatant, we used Biotechne Quantikine ELISA kit Cat #DPRL00. According to manufacturer's instructions, we measured secreted PRL in cells supernatant based on standard curve plotted using different concentrations of human recombinant PRL. Sensitivity of human PRL ELISA kit is 0.264 ng/mL.

Anti-dsDNA was assayed by ELISA using a kit from Chondrex (#3031). To ensure consistent values between assays, we diluted and then stored samples in assay dilution buffer for >24 h at −20° C. before assay.

### PRL Neutralization Assay

Ten thousand cells were seeded per well in 96-well plates and incubated in Complete RPMI containing different concentrations of normal rabbit serum or rabbit anti-PRL (NIDDK standard, AFP55762089) (0, 1:1600, 1:800, 1:400, 1:200, 1:100, 1:50 dilution) at 37° C. for 48 h. Twenty μl of MTS (Promega) was added to 100 μl of media containing cells and incubated for 3 h. Absorbance was measured at 490 nm with a TECAN Infinite M1000 Pro microplate reader.

### LFPRLR SMO Treatment Efficacy Studies in CDX Models of Human B-Cell Malignancies

Immune-deficient NSG mice were used as transplant recipients in the human B-cell malignancy CDX models. Treatment with SMOs in the NSG CDX-recipient mice was conducted as described above in Example 1 for the primary mouse models of SLE and DLBCL, except that the treatment duration was shorter to ensure tumor size remained within guidelines in the control SMO treated animals. Transplant recipient CDX mice were euthanized following the stringent guidelines on tumor volume and health of mice laid down by IACUC of City of Hope.

Six female immune-deficient NSG mice (Jackson Laboratories) received subcutaneous injections of 7×10⁶ malignant B cells suspended in 200 μL matrigel (BD Biosciences) in their right flank. Cell lines were pre-treated in vitro for 3 h with either control SMO or LFPRLR SMO at their respective IC50 concentrations. Alzet minipumps delivering the SMOs were implanted on the left scapula of each mouse. Tumor burden was assessed twice weekly by caliper measurement. Tumor volume was calculated using the formula: V=½(length×width²). Mice were euthanized after 15 days of treatment.

The entire content of Taghi Khani, et al. Commun Biol 6, 295 (2023) is incorporated by reference herein.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of treating or preventing a B cell mediated disorder in a subject in need thereof, comprising:
administering a splice modulating oligonucleotide (SMO) to the subject,
wherein the SMO inhibits B cell expression of the long form PRLR (LF PRLR) and/or IF PRLR; and/or reduces the relative B cell expression of LF/IF PRLR compared to B cell expression of one or more short form prolactin receptors (SF PRLR).

2. The method of claim 1, wherein the B cell mediated disorder is a B cell malignancy.

3. The method of claim 2, wherein the B cell malignancy is diffuse large B cell lymphoma (DLBCL), Burkitt's Lymphoma (BL), follicular lymphoma, B cell chronic lymphocytic leukemia (CLL), or B cell acute lymphoblastic leukemia (B-ALL).

4. The method of claim 1, wherein the B cell mediated disorder is systemic lupus erythematosus (SLE), rheumatoid arthritis (RA), or Sjögren's syndrome (SS).

5. The method of claim 1, which reduces the subject's likelihood of developing a B cell malignancy, wherein the subject has an autoimmune disease or has premalignant B cell clone(s).

6-11. (canceled)

12. The method of claim 1, wherein the oligonucleotide is a morpholino oligonucleotide.

13. The method of claim 1, wherein the oligonucleotide comprises a nucleic acid sequence having at least about 85% sequence identity to GCCCTTCTATTAAAACACAGACACA (SEQ ID NO:1), or GCCCTTCTATTGAAACACAGATACA (SEQ ID NO:2).

14. The method of claim 13, wherein the oligonucleotide comprises the nucleic acid sequence of SEQ ID NO:1 or SEQ ID NO:2.

15. (canceled)

16. The method of claim 1, wherein the oligonucleotide is operably linked to a cell penetrating moiety, or the oligonucleotide is comprised within an intracellular delivery system.

17-18. (canceled)

19. The method of claim 1, wherein the administration reduces B cell expression of activation-induced cytidine deaminase (AID), anti-apoptotic factor BCL-2, and/or oncoprotein c-Myc.

20. The method of claim 1, wherein LF/IF PRLR or total PRLR is overexpressed in a peripheral blood mononuclear cells (PBMCs) sample of the subject prior to the administration of the SMO; and/or wherein B cells overexpress LF/IF PRLR or total PRLR in a sample of the subject prior to the administration of the SMO.

21. (canceled)

22. The method of claim 1, wherein:
1) the number of B cells in a sample of the subject is reduced;
2) the number of plasma cells in a sample of the subject is reduced;
3) the number of plasmablast cells in a sample of the subject is reduced;
4) the number of precursor B cells, in a sample of the subject, is reduced;
5) the number of follicular (FO) B cells in a sample of the subject is reduced; and/or
6) the number of B cells that have long BCR and/or Ig CDR3 having >20 aa in length, in a sample of the subject, is reduced.

23-33. (canceled)

34. The method of claim 1, wherein the number of normal, non-autoreactive plasma cells in a sample of the subject is not reduced.

35. The method of claim 1, wherein:
1) the level of autoreactive antibodies in a sample of the subject is reduced;
2) the level of anti-nuclear antibodies (ANA) in a sample of the subject is reduced;
3) the level of anti-DNA antibodies in a sample of the subject is reduced; and/or
4) the level of anti-histone antibodies in a sample of the subject is reduced.

36-40. (canceled)

41. The method of claim 1, further comprising administering one or more additional therapeutic agents or therapies to the subject, optionally, wherein the one or more additional therapeutic agents or therapies are selected from the group consisting of cyclophosphamide, doxorubicin, vincristine, prednisone, an anti-B cell antibody, an anti-B cell CAR-T, a NK cell-based therapy, a checkpoint inhibitor, stem cell transplantation, a nonsteroidal anti-inflammatory drug (NSAID), a corticosteroid, an immunosuppressant, an antimalarial drug, an anti-B cell activating factor (BAFF) antibody, a Myc inhibitor, a BCL2 inhibitor, an anti-psychotic, a proteasome inhibitor, anti-plasma cell agent, and combinations thereof.

42. (canceled)

43. The method of claim 1, further comprising detecting the expression level of prolactin and/or the expression level of LF/IF PRLR or total PRLR in a sample obtained from the subject prior to administration of the SMO.

44-45. (canceled)

46. A method of identifying a subject as a candidate for treatment with a SLE therapy, comprising detecting the expression level of LF/IF PRLR or total PRLR in a sample from the subject, wherein the subject is identified as a candidate for treatment when the expression level of LF/IF PRLR or total PRLR is higher than a control reference level.

47. A method of identifying a subject as having a higher likelihood of developing a B cell malignancy or as a candidate for treatment with a B cell malignancy therapy, the method comprising detecting the expression level of prolactin or LF/IF PRLR in a sample from the subject, wherein the subject is identified as having a higher likelihood of developing a B cell malignancy or as a candidate for treatment when the expression level of prolactin or LF/IF PRLR is higher than a control reference level.

48. (canceled)

49. The method of claim 47, wherein the subject has an autoimmune disorder and the B cell malignancy is DLBCL, CLL or B-ALL.

50-70. (canceled)

71. The method of claim 2, wherein the B cell malignancy is B cell lymphoma.
